(19) United States
(12) Patent Application Publication
(10) Pub. No.: US 2005/0070508 A1
(43) Pub. Date: Mar. 31, 2005

(54) Napthalene carboxamides and their derivatives useful as new anti-angiogenic agents

(75) Inventors: Jihong Lou, San Diego, CA (US); Robert Steven Kania, San Diego, CA (US); Mingying He, San Diego, CA (US); Ru Zhou, Carlsbad, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/924,528

(22) Filed: Aug. 23, 2004

Related U.S. Application Data
(60) Provisional application No. 60/499,261, filed on Aug. 29, 2003.

(51) Int. Cl. A61K 31/655; A61K 31/519; A61K 31/165

(52) U.S. Cl. 514/130; 514/232.2; 514/567; 514/596; 514/151; 514/617; 514/616

(57) ABSTRACT

The invention relates to compounds represented by Formula (I): 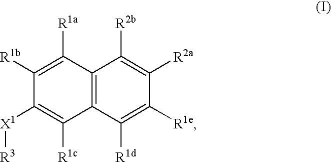
and to prodrugs thereof, pharmaceutically acceptable salts or solvates of said compounds or said prodrugs, wherein each of R^{1a-d}, R^{2a-b}, R³, and X¹ are defined herein. The invention also relates to pharmaceutical compositions containing the compounds of Formula (I) and to methods of treating hyperproliferative disorders in a mammal by administering compounds of Formula (I).

### BACKGROUND OF THE INVENTION

This application claims priority under 35 U.S.C. § 119(e) to U.S. Provisional Application No. 60/499,261, filed Aug. 29, 2003, and is hereby incorporated by reference.

This invention relates to novel naphthalene analogs and derivatives thereof, including pharmaceutically acceptable derivatives, such as salts, prodrugs, solvates, and metabolites. The compounds of the present invention inhibit the activity of receptor kinases such as VEGFR and PDGRF that are required for cell growth and differentiation and angiogenesis. Particularly, the compounds in this invention inhibit VEGFR/KDR and therefore are useful for treatment of diseases and conditions that are associated with VEGFR/KDR activity, e.g., cancer and ophthalmic diseases such as age-related macular degeneration and diabetic retinopathy. This invention also relates to a method of using such compounds in the treatment of hyperproliferative diseases in mammals, especially humans, and to pharmaceutical compositions containing such compounds.

A cell may become cancerous by virtue of the transformation of a portion of its DNA into an oncogene (i.e., a gene that upon activation leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are large enzymes that span the cell membrane and possess an extracellular binding domain for growth factors, a transmembrane domain, and an intracellular portion that functions as a kinase to phosphorylate a specific tyrosine residue in proteins and hence to influence cell proliferation. Tyrosine kinases may be classified as growth factor receptor (e.g. EGFR, PDGFR, FGFR and erbB2) or non-receptor (e.g. c-src and bcr-abl) kinases. Such kinases may be aberrantly expressed in common human cancers such as breast cancer, gastrointestinal cancers such as colon, rectal or stomach cancer, leukemia, and ovarian, bronchial or pancreatic cancer. Aberrant erbB2 activity has been implicated in breast, ovarian, non-small cell lung, pancreatic, gastric and colon cancers. Studies indicate that epidermal growth factor receptor (EGFR) is mutated or overexpressed in many human cancers such as brain, lung, squamous cell, bladder, gastric, breast, head and neck, oesophageal, gynecological and thyroid cancers. Thus, inhibitors of receptor tyrosine kinases may be useful as selective inhibitors of the growth of mammalian cancer cells.

EGFR inhibitors may be useful in the treatment of pancreatitis and kidney disease (such as proliferative glomerulonephritis and diabetes-induced renal disease), and may reduce successful blastocyte implantation and therefore may be useful as a contraceptive. See PCT international application publication number WO 95/19970 (published Jul. 27, 1995), hereby incorporated by reference in its entirety.

Polypeptide growth factors, such as vascular endothelial growth factor (VEGF) having a high affinity to the human kinase insert-domain-containing receptor (KDR) or the murine fetal liver kinase 1 (FLK-1) receptor have been associated with the proliferation of endothelial cells and more particularly vasculogenesis and angiogenesis. See PCT international application publication number WO 95/21613 (published Aug. 17, 1995), hereby incorporated by reference in its entirety. Agents that are capable of binding to or modulating the KDR/FLK-1 receptor may be used to treat disorders related to vasculogenesis or angiogenesis, such as diabetes, diabetic retinopathy, age related macular degeneration, hemangioma, glioma, melanoma, Kaposi's sarcoma and ovarian, breast, lung, pancreatic, prostate, colon and epidermoid cancer.

Compounds and methods that reportedly can be used to treat hyperproliferative diseases are disclosed in the following patents and applications: U.S. Pat. No. 6,534,524, issued Mar. 18, 2003, U.S. Pat. No. 6,531,491, issued Mar. 11, 2003, PCT International Patent Application publication number WO 00/38665 (published Jul. 6, 2001), PCT International Patent Application publication number WO 97/49688 (published Dec. 31, 1997), PCT International Patent Application publication number WO 98/23613 (published Jun. 4, 1998), U.S. Pat. No. 6,071,935 issued Jun. 6, 2000, PCT International Patent Application publication number WO 96/30347 (published Oct. 3, 1996), PCT International Patent Application publication number WO 96/40142 (published Dec. 19, 1996), PCT International Patent Application publication number WO 97/13771 (published Apr. 17, 1997), PCT International Patent Application publication number WO 95/23141 (published Aug. 31, 1995), PCT International Patent Application publication number WO 03/006059 (published Jan. 23, 2003), PCT International Patent Application publication number WO 03/035047 (published May 1, 2003), PCT International Patent Application publication number WO 02/064170 (published Aug. 22, 2002), PCT International Patent Application publication number WO 02/41882 (published May 30, 2002), PCT International Patent Application publication number WO 02/30453 (published Apr. 18, 2002), PCT International Patent Application publication number WO 01/85796 (published Nov. 15, 2001), PCT International Patent Application publication number WO 01/74360 (published Oct. 11, 2001), PCT International Patent Application publication number WO 01/74296 (published Oct. 11, 2001), PCT International Patent Application publication number WO 01/70268 (published Sep. 27, 2001), European Patent Application publication number EP 1086705 (published Mar. 28, 2001), and PCT International Patent Application publication number WO 98/51344 (published Nov. 19, 1998). The foregoing patents and applications are each incorporated herein by reference in their entirety for all purposes.

### SUMMARY OF THE INVENTION

Described herein are compounds capable of modulating the activity of receptor kinases such as VEGFR and PDGRF and methods for utilizing such modulation in the treatment of cancer and other proliferative disorders. Also described are carbamate compounds that mediate and/or inhibit the activity of protein kinases, and pharmaceutical compositions containing such compounds. Also described are therapeutic or prophylactic use of such compounds and compositions, and methods of treating cancer as well as other diseases associated with unwanted angiogenesis and/or cellular proliferation, by administering effective amounts of such compounds.

In one aspect are novel quinoline compounds. In another aspect provided are compounds that modulate the activity of receptor kinases such as KDR/VEGFR2 kinase in vitro and/or in vivo. According to a further aspect, provided are compounds that can selectively modulate the activity of receptor kinases such as KDR/VEGFR2 kinase. In yet another aspect, provided are pharmaceutical compositions of such VEGFR2-modulating compounds, including pharmaceutically acceptable prodrugs, pharmaceutically active metabolites, or pharmaceutically acceptable salts thereof. According to yet another aspect, provided are syntheses schemes for the preparation of such VEGFR2-modulating compounds, and pharmaceutically acceptable prodrugs, pharmaceutically active metabolites, or pharmaceutically acceptable salts thereof. In yet another aspect, methods are provided for modulating KDR/VEGFR2 kinase which comprise contacting the VEGFR2-modulating compounds, or pharmaceutically acceptable prodrugs, pharmaceutically active metabolites, or pharmaceutically acceptable salts thereof, described herein, with KDR/VEGFR2 kinase. In yet another aspect, methods are provided for treating patients comprising administering a therapeutically effective amount of a VEGFR2-modulating compound, or a pharmaceutically acceptable prodrug, pharmaceutically active metabolite, or pharmaceutically acceptable salt thereof. In yet another aspect, the present invention comprises combination therapies involving administration of an anti-neoplastic agent and an effective amount of a VEGFR2-modulating compound, or a pharmaceutically acceptable prodrug, pharmaceutically active metabolite, or pharmaceutically acceptable salt thereof.

In one aspect are compounds represented by Formula (I): 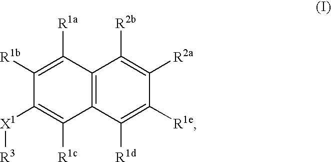
wherein
(a) one of R^{2a} and R^{2b} is —C(O)NHR⁴ and the other is R^{1f};
(b) each of R^{1a}, R^{1b}, R^{1c}, R^{1d}, R^{1e} and R^{1f} is independently selected from the group consisting of H, halogen, OH, NH₂, N₃, NO₂, (C₁-C₆)alkoxyl, (C₁-C₆)alkyl, (C₁-C₆)fluoroalkoxyl, and (C₁-C₆)fluoroalkyl;
(c) X¹ is O or S;
(d) R³ is either H or a moiety selected from the group consisting of —(CZ¹Z²)ⱼCN, —(CZ¹Z²)ⱼ—(C₃-C₈)cycloalkyl, —(CZ¹Z²)ⱼ—(C₅-C₈)cycloalkenyl, (C₂-C₆)alkenyl, (C₂-C₆)alkynyl, —(CZ¹Z²)ⱼ-aryl, —(CZ¹Z²)ⱼ-heterocyclyl, and (C₁-C₈)alkyl, where j is 0, 1, 2, or 3, and wherein when j is 2 or 3, each CZ¹Z² unit may be the same or different, and wherein Z¹ and Z² are independently selected from the group consisting of H, F, and (C₁-C₆)alkyl, or wherein Z¹ and Z² taken together can optionally form a (C₃-C₈)carbocyclyl, or two Z¹ groups on adjacent atoms taken together can form a (C₃-C₈)carbocyclyl;
(e) R⁴ is either H or a moiety selected from the group consisting of —(CZ¹Z²)ⱼCN, —(CZ¹Z²)ⱼ—(C₃-C₈)cycloalkyl, —(CZ¹Z²)ⱼ—(C₅-C₈)cycloalkenyl, (C₂-C₆)alkenyl, (C₂-C₆)alkynyl, —(CZ¹Z²)ⱼ-aryl, —(CZ¹Z²)ⱼ-heterocyclyl, and (C₁-C₈)alkyl, where j is 0, 1, 2, or 3, and wherein when j is 2 or 3, each CZ¹Z² unit may be the same or different, and wherein Z¹ and Z² are independently selected from the group consisting of H, F, and (C₁-C₆)alkyl, or wherein Z¹ and Z² taken together can optionally form a carbocyclyl, or two Z¹ groups on adjacent atoms taken together can form a (C₃-C₈)carbocyclyl;
(f) wherein each R³ and R⁴ may be optionally substituted on any carbon atom with a hydrogen atom, with 1-3 independently selected Y groups;
(g) each Y is:
   (i) independently selected from the group consisting of halogen, cyano, nitro, tetrazolyl, guanidino, amidino, methylguanidino, azido, C(O)Z³, —CF₃, —CF₂CF₃, —CH(CF₃)₂, —C(OH)(CF₃)₂, —OCF₃, —OCF₂H, —OCF₂CF₃, —OC(O)NH₂, —OC(O)NHZ³, —OC(O)NZ³Z⁴, —NHC(O)Z³, —NHC(O)NH₂, —NHC(O)NHZ³, —NHC(O)NZ³Z⁴, —C(O)OH, —C(O)OZ³, —C(O)NH₂, —C(O)NHZ³, —C(O)NZ³Z⁴, —P(O)₃H₂, —P(O)₃ (Z³)₂, —S(O)₃H, —S(O)ₘZ³, -Z³, —OZ³, —OH, —NH₂, —NHZ³, —NZ³Z⁴, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C¹-C₆)haloalkoxyl, —(CZ⁵Z⁶)ᵣNH₂, —(CZ⁵Z⁶)ᵣNHZ₁, (CZ⁵Z⁶)ᵣNZ³Z⁴, X²(CZ⁵Z⁶)ᵣ—(C₃-C₈)cycloalkyl, —X²(CZ⁵Z⁶)ᵣ—(C₅-C₈)cycloalkenyl, —X²(CZ⁵Z⁶)ᵣ-aryl, —X²(CZ⁵Z⁶)ᵣ-heterocyclyl, and —S(O)ₘ(CF₂)_{q}CF₃, wherein m is 0, 1 or 2; q is 0, 1, 2, 3, 4, or 5; r is 1, 2, 3, or 4; X² is O, S, NH, —C(O)—, —C(O)NH—, or —C(O)O—; Z³ and Z⁴ are independently selected from the group consisting of (C₁-C₁₂) alkyl, (C₂-C₁₂) alkenyl, (C₂-C₁₂) alkynyl, (C₃-C₈) cycloalkyl, (C₅-C₈) cycloalkenyl, (C₆-C₁₄) aryl, 5 to 14 membered heterocyclyl, 7 to 15 membered aryl alkyl, and 5 to 14 membered heteroaryl alkyl; and Z⁵ and Z⁶ are independently selected from the group consisting of hydrogen, fluorine, (C₁-C₁₂) alkyl, (C₆-C₁₄) aryl, 5 to 14 membered heteroaryl, 7 to 15 membered aryl alkyl, and 5 to 14 membered heteroaryl alkyl; or
   (ii) any two Y groups attached to adjacent carbon atoms may be selected together to be —O[C(Z⁵)(Z⁶)]ᵣO— or —O[C(Z⁵)(Z⁶)]ᵣ₊₁-; or
   (iii) any two Y groups attached to the same or adjacent carbon atoms may be selected together to form a (C₃-C₈)carbocyclyl or (C₃-C₈)heterocyclyl; and wherein any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C¹-C₄)alkyl, (C₁-C₄)alkoxyl and —N[(C₁-C₄)alkyl][(C₁-C₄)alkyl]; or an N-oxide, pharmaceutically acceptable prodrug, pharmaceutically active metabolite, pharmaceutically acceptable salt, or pharmaceutically acceptable solvate thereof.

In a further embodiment are compounds having the structure of Formula (I), wherein R^{2a} is H and R^{2b} is —C(O)NHR⁴. In yet a further embodiment are compounds having the structure of Formula (I), wherein R^{2a} is H and R^{2b} is —C(O)NHR⁴; and X is O. In yet a further embodiment are compounds having the structure of Formula (I), wherein R^{2a} is H and R^{2b} is —C(O)NHR⁴; X is O; and one of R^{1a}, R^{1b}, R^{1c}, R^{1d}, R^{1e} and R^{1f} is selected from the group consisting of halogen, (C₁-C₆)alkoxyl, (C₁-C₆)alkyl, (C₁-C₆)fluoroalkoxyl, and (C₁-C₆)fluoroalkyl and the other five of R^{1a}, R^{1b}, R^{1c}, R^{1d}, R^{1e} and R^{1f} are H.

In yet a further embodiment are compounds having the structure of Formula (I), wherein R^{2a} is H and R^{2b} is —C(O)NHR⁴; X is O; and only one of R^{1a}, R^{1b}, R^{1c}, R^{1d}, R^{1e} and R^{1f} is F and the other five of R^{1a}, R^{1b}, R^{1c}, R^{1d}, R^{1e} and R^{1f} are H. Furthermore, in such an embodiment are compounds in which R³ is a moiety selected from the group consisting of —(C₃-C₈)cycloalkyl, —(C₅-C₈)cycloalkenyl, —(C₃-C₈)aryl, and —(C₃-C₈)heterocyclyl; wherein each R³ may be optionally substituted on any carbon atom with a hydrogen atom, with 1-3 independently selected Y groups.

In yet a further embodiment are compounds having the structure of Formula (I), wherein R^{2a} is H and R^{2b} is —C(O)NHR⁴; and R³ is a moiety selected from the group consisting of —(C₃-C₈)aryl and —(C₃-C₈)heterocyclyl; wherein each R³ may be optionally substituted on any carbon atom with a hydrogen atom, with 1-3 independently selected Y groups.

In yet a further embodiment are compounds having the structure of Formula (I), wherein R^{2a} is H and R^{2b} is —C(O)NHR⁴; X is O; only one of R^{1a}, R^{1b}, R^{1c}, R^{1d}, R^{1e} and R^{1f} is F and the other five of R^{1a}, R^{1b}, R^{1c}, R^{1d}, R^{1e} and R^{1f} are H; and R³ is a moiety selected from the group consisting of —(C₃-C₈)aryl and —(C₃-C₈)heterocyclyl; wherein each R³ may be optionally substituted on any carbon atom with a hydrogen atom, with 1-3 independently selected Y groups. In a further embodiment of such compound are compounds wherein each Y group on R³ is selected from the group consisting of halogen, C(O)Z³, —OC(O)NHZ³, —OC(O)NZ³Z⁴, —NHC(O)Z³, —C(O)OZ³, —C(O)NHZ³, —C(O)NZ³Z⁴, -Z³, —OZ³, —NHZ³, —NZ³Z⁴, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxyl, —(CZ⁵Z⁶)ᵣNZ³Z⁴, —X²(CZ⁵Z⁶)ᵣ—(C₃-C₈)cycloalkyl, —X²(CZ⁵Z⁶)ᵣ—(C₅-C₈)cycloalkenyl, —X²(CZ⁵Z⁶)-aryl, and —X²(CZ⁵Z⁶)ᵣ-heterocyclyl, r is 1, 2, 3, or 4; X² is O, S, NH, —C(O)—, —C(O)NH—, or —C(O)O—; Z³ and Z⁴ are independently selected from the group consisting of (C₁-C₁₂) alkyl, (C₂-C₁₂) alkenyl, (C₂-C₁₂) alkynyl, (C₃-C₈) cycloalkyl, (C₅-C₈) cycloalkenyl, (C₆-C₁₄) aryl, 5 to 14 membered heterocyclyl, 7 to 15 membered aryl alkyl, and 5 to 14 membered heteroaryl alkyl; and Z⁵ and Z⁶ are independently selected from the group consisting of hydrogen, fluorine, (C₁-C₁₂) alkyl, (C₆-C₁₄) aryl, 5 to 14 membered heteroaryl, 7 to 15 membered aryl alkyl, and 5 to 14 membered heteroaryl alkyl. In yet a further embodiment are compounds having the structure of Formula (I), wherein R^{2a} is H and R^{2b} is —C(O)NHR⁴; X is O; only one of R^{1a}, R^{1b}, R^{1c}, R^{1d}, R^{1e} and R^{1f} is F and the other five of R^{1a}, R^{1b}, R^{1c}, R^{1d}, R^{1e} and R^{1f} are H; R³ is a moiety, optionally substituted with 1-3 independently selected Y groups, selected from the group consisting of -aryl and -heterocyclyl; and R⁴ is a moiety, optionally substituted with 1-3 independently selected Y groups, selected from the group consisting of —(CZ¹Z²)ⱼ—(C₃-C₈)cycloalkyl, —(CZ¹Z²)ⱼ-aryl, —(CZ¹Z²)ⱼ-heterocyclyl, and (C₁-C₈)alkyl, wherein each Z¹ and Z² of each —CZ¹Z²— are independently H or F. In a further refinement of such compounds, R⁴ is a moiety, optionally substituted with 1-3 independently selected Y groups, selected from the group consisting of —(CH₂)ⱼ—(C₃-C₈)cycloalkyl, —(CH₂)ⱼ-aryl, —(CH₂)ⱼ-heterocyclyl, and (C₁-C₈)alkyl.

In yet a further embodiment are compounds having the structure of Formula (I), wherein R^{2a} is H and R^{2b} is —C(O)NHR⁴; and R³ is selected from the group consisting of (a) a 5-membered aromatic, monocyclic heterocyclyl having 1 to 4 heteroatoms selected from the group consisting of O, S, and N, optionally substituted with 1-3 independently selected Y groups; and (b) a 6-membered aromatic, monocyclic heterocyclyl having 1 to 4 heteroatoms selected from the group consisting of O, S, and N, optionally substituted with 1-3 independently selected Y groups; wherein heterocyclyl (a) and (b) may be optionally fused to another carbocyclyl or heterocyclyl to form a fused bicyclic ring structure.

In yet a further embodiment are compounds having the structure of Formula (I), wherein R^{2a} is H and R^{2b} is —C(O)NHR⁴; X is O; only one of R^{1a}, R^{1b}, R^{1c}, R^{1d}, R^{1e} and R^{1f} is F and the other five of R^{1a}, R^{1b}, R^{1c}, R^{1d}, R^{1e} and R^{1f} are H; R⁴ is a moiety, optionally substituted with 1-3 independently selected Y groups, selected from the group consisting of —(CH₂)ⱼ—(C₃-C₈)cycloalkyl, —(CH₂)ⱼ-aryl, —(CH₂)ⱼ-heterocyclyl, and (C₁-C₈)alkyl; and R³ is selected from the group consisting of (a) a 5-membered aromatic, monocyclic heterocyclyl having 1 to 4 heteroatoms selected from the group consisting of O, S, and N, optionally substituted with 1-3 independently selected Y groups; and (b) a 6-membered aromatic, monocyclic heterocyclyl having 1 to 4 heteroatoms selected from the group consisting of O, S, and N, optionally substituted with 1-3 independently selected Y groups; wherein heterocyclyl (a) and (b) may be optionally fused to another carbocyclyl or heterocyclyl to form a fused bicyclic ring structure.

In another embodiment are compounds having the structure of Formula (I), more specifically represented by Formula (II): 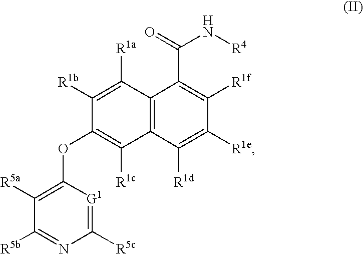
wherein
(a) G¹ is N or CR^{5d};
(b) each of R^{5a} and R^{5b} is independently H, halogen, or a moiety selected from the group consisting of —X³(CH₂)ₖ—(C₃-C₈)cycloalkyl, —X³(CH₂)ₖ—(C₅-C₈)cycloalkenyl, —X³(C₂-C₆)alkenyl, —X³(C₂-C₆)alkynyl, —X³(CH₂)ₖ-aryl, —X³(CH₂)ₖ-heterocyclyl, and —X³(C₁-C₈)alkyl, where k is 0, 1, 2, or 3, and wherein X³ is O, S, NH, —C(O)—, —C(O)NH—, or —C(O)O—; or optionally R^{5a} and R^{5b} taken together form a group, optionally substituted with 1-3 independently selected Y groups, selected from (C₃-C₈)cycloalkyl, (C₅-C₈)cycloalkenyl, (C₃-C₈)aryl, and (C₃-C₈)heterocyclyl; and
(c) each of R^{5c} and R^{5d} is independently H or halogen.

In yet a further embodiment are compounds having the structure of Formula (II), wherein one of R^{1a}, R^{1b}, R^{1c}, R^{1d}, R^{1e} and R^{1f} is selected from the group consisting of halogen, (C₁-C₆)alkoxyl, (C₁-C₆)alkyl, (C₁-C₆)fluoroalkoxyl, and (C₁-C₆)fluoroalkyl and the other five of R^{1a}, R^{1b}, R^{1c}, R^{1d}, R^{1e} and R^{1f} are H. In a further embodiment of such compounds are compounds wherein one of R^{1a}, R^{1b}, R^{1c}, R^{1d}, R^{1e} and R^{1f} is F and the other five of R^{1a}, R^{1b}, R^{1c}, R^{1d}, R^{1e} and R^{1f} are H.

In yet a further embodiment are compounds having the structure of Formula (II), wherein R^{5a} and R^{5b} taken together form a group, optionally substituted with 1-3 independently selected Y groups, selected from (C₃-C₈)cycloalkyl, (C₅-C₈)cycloalkenyl, aryl, and heterocyclyl. In a further embodiment of such compounds are compounds wherein R^{5a} and R^{5b} taken together form an aryl group, optionally substituted with 1-3 independently selected Y groups.

In yet a further embodiment are compounds having the structure of Formula (II), wherein R^{5a} and R^{5b} taken together form an aryl group, optionally substituted with 1-3 independently selected Y groups; and wherein each Y group on the aryl group formed from R^{5a} and R^{5b} is selected from the group consisting of halogen, C(O)Z³, —OC(O)NHZ³, —OC(O)NZ³Z⁴, —NHC(O)Z³, —C(O)OZ³, —C(O)NHZ³, —C(O)NZ³Z⁴, -Z³, —OZ³, —NHZ³, —NZ³Z⁴, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxyl, —(CZ⁵Z⁶)ᵣNZ³Z⁴, X²(CZ⁵Z⁶)ᵣ—(C₃-C₈)cycloalkyl, —X²(CZ⁵Z⁶)ᵣ—(C₅-C₈)cycloalkenyl, —X²(CZ⁵Z⁶)ᵣ-aryl, and —X²(CZ⁵Z⁶)ᵣ-heterocyclyl.

In yet a further embodiment are compounds having the structure of Formula (II), wherein R^{5a} and R^{5b} taken together form an aryl group, optionally substituted with 1-3 independently selected Y groups; wherein each Y group on the aryl group formed from R^{5a} and R^{5b} is selected from the group consisting of halogen, C(O)Z³, —OC(O)NHZ³, —OC(O)NZ³Z⁴, —NHC(O)Z³, —C(O)OZ³, —C(O)NHZ³, —C(O)NZ³Z⁴, -Z³, —OZ³, —NHZ³, —NZ³Z⁴, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxyl, —(CZ⁵Z⁶)ᵣNZ³Z⁴, —X²(CZ⁵Z⁶)ᵣ—(C₃-C₈)cycloalkyl, —X²(CZ⁵Z⁶)ᵣ—(C₅-C₈)cycloalkenyl, —X²(CZ⁵Z⁶)ᵣ-aryl, and —X²(CZ⁵Z⁶)ᵣ-heterocyclyl; and wherein R⁴ is a moiety, optionally substituted with 1-3 independently selected Y groups, selected from the group consisting of —(CZ¹Z²)ⱼ—(C₃-C₈)cycloalkyl, —(CZ¹Z²)ⱼ-aryl, —(CZ¹Z²)ⱼ-heterocyclyl, and (C₁-C₈)alkyl, wherein each Z¹ and Z² of each —CZ¹Z²— are independently H or F.

In yet a further embodiment are compounds having the structure of Formula (II), wherein R^{5a} and R^{5b} taken together form an aryl group, optionally substituted with 1-3 independently selected Y groups; wherein each Y group on the aryl group formed from R^{5a} and R^{5b} is selected from the group consisting of halogen, C(O)Z³, —OC(O)NHZ³, —OC(O)NZ³Z⁴, —NHC(O)Z³, —C(O)OZ³, —C(O)NHZ³, —C(O)NZ³Z⁴, -Z³, —OZ³, —NHZ³, —NZ³Z⁴, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxyl, —(CZ⁵Z⁶)ᵣNZ³Z⁴, —X²(CZ⁵Z⁶)ᵣ—(C₃-C₈)cycloalkyl, —X²(CZ⁵Z⁶)ᵣ—(C₅-C₈)cycloalkenyl, —X²(CZ⁵Z⁶)ᵣ-aryl, and —X²(CZ⁵Z⁶)ᵣ-heterocyclyl; and wherein R⁴ is a moiety, optionally substituted with 1-3 independently selected Y groups, selected from the group consisting of —(CH₂)ⱼ—(C₃-C₈)cycloalkyl, —(CH₂)ⱼ-aryl, —(CH₂)ⱼ-heterocyclyl, and (C₁-C₈)alkyl, where j is 0, 1, 2, or 3.

In another embodiment are compounds having the structure of Formula (I), wherein R³ is selected from the group consisting of: 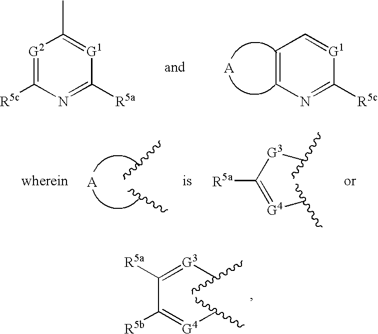
where
(a) G¹ and G² are N or C(R^{5d}), provided only one of G¹ and G² can be N;
(b) G³ and G⁴ are S, N or C(R^{5e}) provided only one of G³ and G⁴ can be S;
(c) each of R^{5a} and R^{5b} is independently H, halogen, or a moiety selected from the group consisting of —X³(CH₂)ₖ—(C₃-C₈)cycloalkyl, —X³(CH₂)ₖ—(C₅-C₈)cycloalkenyl, —X³(C₂-C₆)alkenyl, —X³(C₂-C₆)alkynyl, —X³(CH₂)ₖ-aryl, —X³(CH₂)ₖ-heterocyclyl, and —X³(C₁-C₈)alkyl, where k is 0, 1, 2, or 3, and wherein X³ is O, S, NH, —C(O)—, —C(O)NH—, or —C(O)O—; or optionally R^{5a} and R^{5b} taken together form a group, optionally substituted with 1-3 independently selected Y groups, selected from (C₃-C₆)cycloalkyl, (C₅-C₈)cycloalkenyl, (C₃-C₈)aryl, and (C₃-C₈)heterocyclyl; and
   (d) each of R^{5c}, and R^{5d} and R^{5e} is independently H or halogen. More particularly, in such compounds R³ may be either

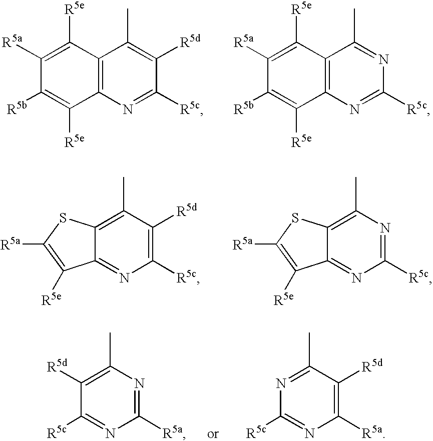

In another embodiment are compounds having the structure of Formula (I) selected from the group consisting of 6-[2-(1-Methyl-1H-imidazol-2-yl)-thieno[3,2-b]pyridin-7-yloxy]-naphthalene-1-carboxylic acid (2-pyrrolidin-1-yl-ethyl)-amide, 6-[7-(2-Piperidin-1-yl-ethoxy)-quinolin-4-yloxy]naphthalene-1-carboxylic acid methylamide, 6-[7-(2-Morpholin-4-yl-ethoxy)-quinolin-4-yloxy]naphthalene-1-carboxylic acid methylamide, N-Methyl-6-{[2-({3-[(methylamino)methyl]pyrrolidin-1-yl}carbonyl)thieno[3,2-b]pyridin-7-yl]oxy}-1-naphthamide, 6-[2-(Azetidine-1-carbonyl)-thieno[3,2-b]pyridin-7-yloxy]-naphthalene-1-carboxylic acid (2-morpholin-4-yl-ethyl)-amide, N-Cyclopropyl-6-[(2-{[(3R)-3-(dimethylamino)pyrrolidin-1-yl]carbonyl}thieno[3,2-b]pyridin-7-yl)oxy]-1-naphthamide, 6-[2-(1-Methyl-1H-imidazol-2-yl)-thieno[3,2-b]pyridin-7-yloxy]-naphthalene-1-carboxylic acid (3-morpholin-4-yl-propyl)-amide, N-[3-(Dimethylamino)propyl]-N-methyl-7-({5-[(methylamino) carbonyl]-2-naphthyl}oxy)thieno[3,2-b]pyridine-2-carboxamide, 5-Fluoro-6-[(2-{[(3S)-3-methoxypyrrolidin-1-yl]carbonyl}thieno[3,2-b]pyridin-7-yl)oxy]-N-methyl-1-naphthamide, 6-(7-Methoxy-quinolin-4-yloxy)-naphthalene-1-carboxylic acid cyclopropylamide, 6-[7-(2-Pyrrolidin-1-yl-ethoxy)-quinolin-4-yloxy]-naphthalene-1-carboxylic acid butylamide, 6-{[2-(1-Methyl-1H-imidazol-2-yl)thieno[3,2-b]pyridin-7-yl]oxy}-N-(2-morpholin-4-ylethyl)-1-naphthamide, 6[(2-{[(3R)-3-Hydroxypyrrolidin-1-yl]carbonyl}thieno[3,2-b]pyridin-7-yl)oxy]-N-(2-morpholin-4-ylethyl)-1-naphthamide, 6-[(2{[(3S)-3-methoxypyrrolidin-1-yl]carbonyl}thieno[3,2-b]pyridin-7-yl)oxy]-N-methyl-1-naphthamide, or a pharmaceutically acceptable prodrug, pharmaceutically active metabolite, pharmaceutically acceptable solvate or pharmaceutically acceptable salt thereof.

In another embodiment are compounds having the structure of Formula (I) selected from the group consisting of: 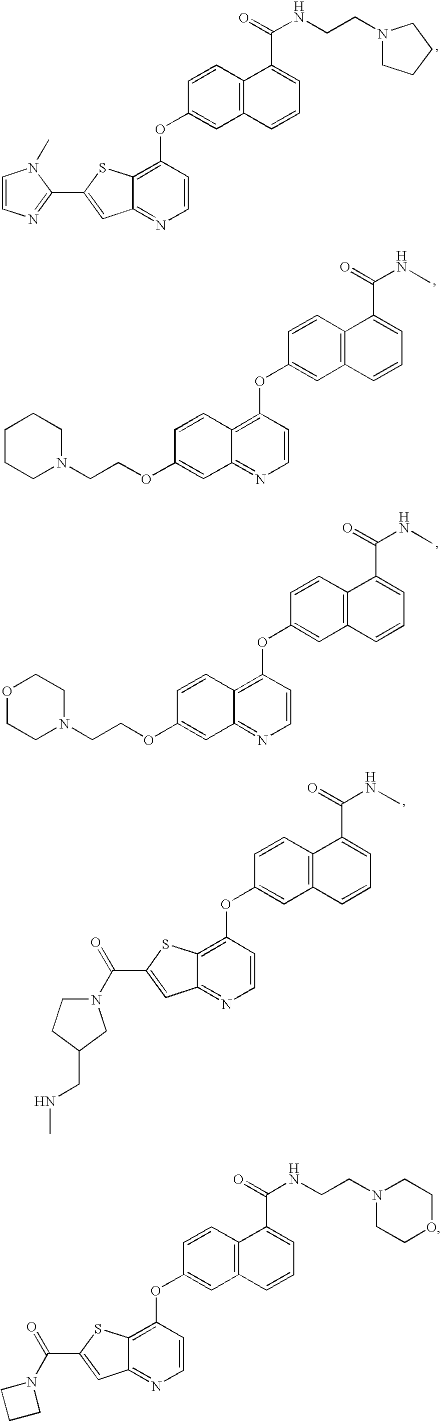 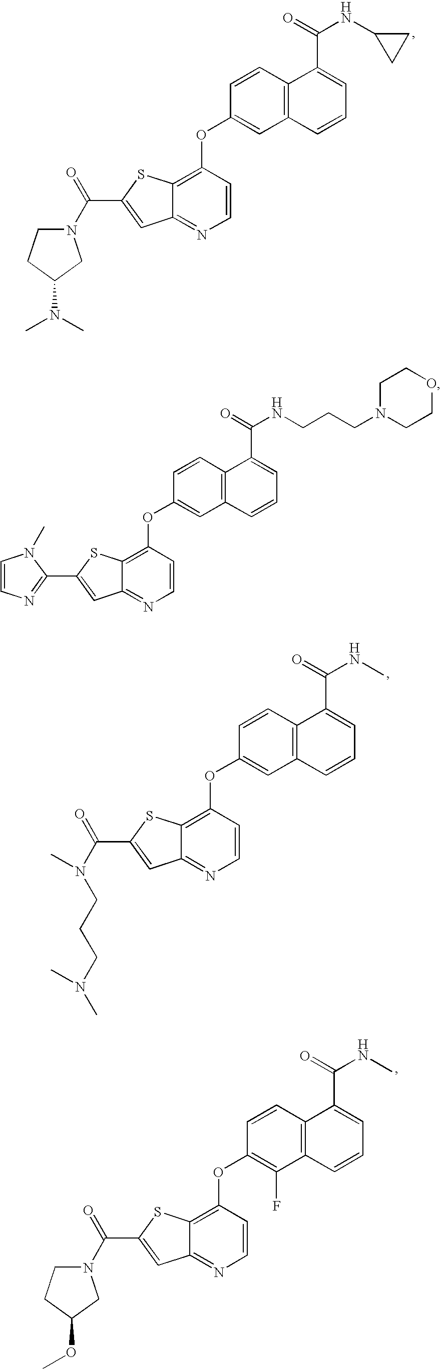 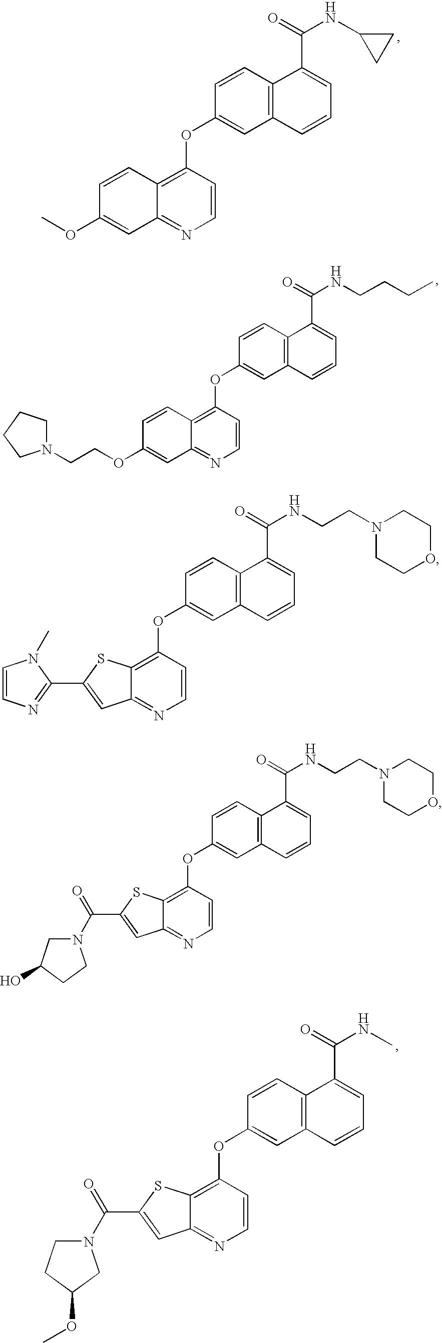
or a pharmaceutically acceptable prodrug, pharmaceutically active metabolite, pharmaceutically acceptable solvate or pharmaceutically acceptable salt thereof.

In another embodiment are methods for producing a compound having the structure of Formula (II), comprising: (a) reacting a carboxylic acid having the structure 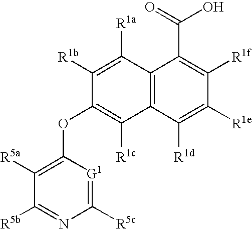
with a chlorinating agent; and
(b) reacting the corresponding product with H₂N—R⁴. In a further embodiment of such methods the chlorinating agent is selected from the group consisting of thionyl chloride, oxalyl chloride, and chlorine. Furthermore, within such methods for producing a compound having the structure of Formula (II) are methods for producing a carboxylic acid having the structure: 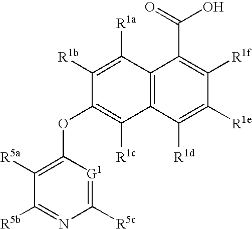
   comprising reacting a compound having the formula 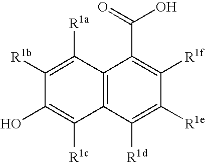
   with a compound having the formula 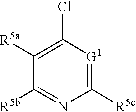
   in the presence of a base.

In another embodiment are methods for producing a compound having the structure of Formula (II), comprising: (a) reacting an amide having the structure 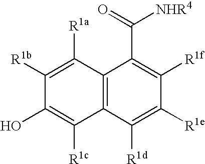
with a compound having the formula 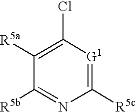
in the presence of a base.

This invention also relates to a pharmaceutical composition for the treatment of abnormal cell growth in a mammal, including a human, comprising an amount of a compound of the formula 1, as defined above, or a pharmaceutically acceptable salt, solvate or prodrug thereof, that is effective in treating abnormal cell growth, and a pharmaceutically acceptable carrier. In one embodiment of said composition, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of a compound of formula 1, as defined above, or a pharmaceutically acceptable salt, solvate or prodrug thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

This invention also relates to a method for the treatment of abnormal cell growth in a mammal, including a human, comprising administering to said mammal an amount of a compound of the formula 1, as defined above, or a pharmaceutically acceptable salt or solvate thereof, that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

This invention also relates to a method for the treatment of a disorder associated with angiogenesis in a mammal, including a human, comprising administering to said mammal an amount of a compound of the formula 1, as defined above, or a pharmaceutically acceptable salt, solvate or prodrug thereof, that is effective in treating said disorder. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of a compound of formula 1, or a pharmaceutically acceptable salt, solvate or prodrug thereof, in combination with an amount of one or more substances selected from anti-tumor agents, anti-angiogenesis agents, signal transduction inhibitors, and antiproliferative agents, which amounts are together effective in treating said abnormal cell growth. Such substances include those disclosed in PCT publication Nos. WO 00/38715, WO 00/38716, WO 00/38717, WO 00/38718, WO 00/38719, WO 00/38730, WO 00/38665, WO 00/37107 and WO 00/38786, all published on Jul. 6, 2000, the disclosures of which are incorporated herein by reference in their entireties for all purposes.

Anti-tumor agents can be used in conjunction with a compound of formula 1 in the methods and pharmaceutical compositions described herein. Examples of anti-tumor agents include mitotic inhibitors, for example vinca alkaloid derivatives such as vinblastine vinorelbine, vindescine and vincristine; colchines allochochine, halichondrine, N-benzoyltrimethyl-methyl ether colchicinic acid, dolastatin 10, maystansine, rhizoxine, taxanes such as taxol (paclitaxel), docetaxel (Taxotere), 2′-N-[3-(dimethylamino)propyl]glutaramate (taxol derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, triethylenemelamine, uracil nitrogen mustard, bis(3-mesyloxypropyl)amine hydrochloride, mitomycin, nitrosoureas agents such as cyclohexyl-chloroethylnitrosourea, methylcyclohexyl-chloroethylnitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxuridine and fludarabine. DNA/RNA antimetabolites, for example, L-alanosine, 5-azacytidine, acivicin, aminopterin and derivatives thereof such as N-[2-chloro-5-[[(2,4-diamino-5-methyl-6-quinazolinyl)methyl]amino]benzoyl]-L-aspartic acid, N-[4-[[(2,4-diamino-5-ethyl-6-quinazolinyl)methyl]amino]benzoyl]-L-aspartic acid, N-[2-chloro-4-[[(2,4-diaminopteridinyl)methyl]amino]benzoyl]-L-aspartic acid, soluble Baker's antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred ant-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of formula 1 in the methods and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R, 3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R, 3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;
   and pharmaceutically acceptable salts, solvates and prodrugs of said compounds.

Signal transduction inhibitors can be used in conjunction with a compound of formula 1 in the methods and pharmaceutical compositions described herein. Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example SU-5416 and SU-6668 (Sugen Inc. of South San Francisco, Calif., USA), can also be combined with a compound of formula 1. VEGF inhibitors are described in, for example, U.S. Pat. No. 6,534,524, issued Mar. 18, 2003, U.S. Pat. No. 6,531,491, issued Mar. 11, 2003, WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome pic), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following United States Patent Applications: 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following United States Provisional Patent Applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing Patent Applications and provisional Patent Applications is herein incorporated by reference in their entirety.

A compound of formula 1 may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Provisional Application 60/113,647 (filed Dec. 23, 1998) which is herein incorporated by reference in its entirety.

The invention also relates to a pharmaceutical composition for the treatment of pancreatitis or kidney disease (including proliferative glomerulonephritis and diabetes-induced renal disease) in a mammal which comprises a therapeutically effective amount of a compound of formula (I), or prodrugs thereof, pharmaceutically active metabolites, pharmaceutically acceptable salts, or pharmaceutically acceptable solvates of said compounds and said prodrugs, and a pharmaceutically acceptable carrier.

The invention also relates to a pharmaceutical composition for the prevention of blastocyte implantation in a mammal which comprises a therapeutically effective amount of a compound of formula (I), or prodrugs thereof, pharmaceutically active metabolites, pharmaceutically acceptable salts, or pharmaceutically acceptable solvates of said compounds and said prodrugs, and a pharmaceutically acceptable carrier.

The invention also relates to a pharmaceutical composition for treating a disease related to vasculogenesis or angiogenesis in a mammal which comprises a therapeutically effective amount of a compound of formula (I), or prodrugs thereof, pharmaceutically active metabolites, pharmaceutically acceptable salts, or pharmaceutically acceptable solvates of said compounds and said prodrugs, and a pharmaceutically acceptable carrier. In one embodiment, said pharmaceutical composition is for treating a disease selected from the group consisting of tumor angiogenesis, chronic inflammatory disease such as rheumatoid arthritis, atherosclerosis, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The invention also relates to a method of treating a hyperproliferative disorder in a mammal which comprises administering to said mammal a therapeutically effective amount of the compound of formula (I), or prodrugs thereof, pharmaceutically active metabolites, pharmaceutically acceptable salts, or pharmaceutically acceptable solvates of said compounds and said prodrugs. In one embodiment, said method relates to the treatment of cancer such as brain, ophthalmic, squamous cell, bladder, gastric, pancreatic, breast, head, neck, oesophageal, prostate, colorectal, lung, renal, kidney, ovarian, gynecological or thyroid cancer. In another embodiment, said method relates to the treatment of a non-cancerous hyperproliferative disorder such as benign hyperplasia of the skin (e.g., psoriasis) or prostate (e.g., BPH).

The invention also relates to a method for the treatment of a hyperproliferative disorder in a mammal which comprises administering to said mammal a therapeutically effective amount of a compound of formula (I), or prodrugs thereof, pharmaceutically active metabolites, pharmaceutically acceptable salts, or pharmaceutically acceptable solvates of said compounds and said prodrugs, in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The treatment of a hyperproliferative disorder in a mammal which comprises administering to said mammal a therapeutically effective amount of a VEGF receptor tyrosine kinase inhibitor may lead to a sustained increase in blood pressure. The compounds of the present invention may be used in conjunction with an anti-hypertensive, such as NORVASC or PROCARDIA XL, commercially available from Pfizer, for use in the treatment of a hyperproliferative disorder in a mammal.

This invention also relates to a pharmaceutical composition for treating a disease related to vasculogenesis or angiogenesis in a mammal comprising (a) therapeutically effective amount of a compound of formula (I), or prodrugs thereof, pharmaceutically active metabolites, pharmaceutically acceptable salts, or pharmaceutically acceptable solvates of said compounds and said prodrugs, (b) a therapeutically effective amount of a compound, prodrug, metabolite, salt or solvate of an inhibitor of tumor necrosis factor alpha, and (c) a pharmaceutically acceptable carrier.

This invention also relates to a pharmaceutical composition for treating a disease related to undesired angiogenesis, endothelial cell migration or endothelial cell proliferation in a mammal comprising (a) therapeutically effective amount of a compound of formula (I), or prodrugs thereof, pharmaceutically active metabolites, pharmaceutically acceptable salts, or pharmaceutically acceptable solvates of said compounds and said prodrugs, (b) a therapeutically effective amount of a compound, prodrug, metabolite, salt or solvate of a NADPH oxidase inhibitor, and (c) a pharmaceutically acceptable carrier.

This invention also relates to a pharmaceutical composition for inhibiting abnormal cell growth in a mammal, including a human, comprising an amount of a compound of formula (I), or prodrugs thereof, pharmaceutically active metabolites, pharmaceutically acceptable salts, or pharmaceutically acceptable solvates of said compounds and said prodrugs, that is effective in inhibiting farnesyl protein transferase, and a pharmaceutically acceptable carrier.

This invention also relates to a pharmaceutical composition for inhibiting abnormal cell growth in a mammal which comprises an amount of a compound of formula (I), or prodrugs thereof, pharmaceutically active metabolites, pharmaceutically acceptable salts, or pharmaceutically acceptable solvates of said compounds and said prodrugs, in combination with an amount of a chemotherapeutic, wherein the amounts of the compound, salt, solvate, or prodrug of formula (I), and of the chemotherapeutic are together effective in inhibiting abnormal cell growth. Many chemotherapeutics are presently known in the art. In one embodiment, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, e.g. anti-androgens.

The compounds described herein may be used in a method for preventing or reducing the growth of tumor cells expressing functional VEGF-1 receptors by administering an effective amount of a small molecule VEGF-1 receptor antagonist to inhibit autocrine stimulation and an effective amount of a compound of Formula (I). Active ingredients in such compositions may be present in free form or in the form of a pharmaceutical acceptable salt and optionally at least one pharmaceutically acceptable carrier.

The compounds described herein also may be used in combination with a selective COX-2-inhibitor for simultaneous, separate or sequential use. The compounds described herein may also be used in combination with a truncated, soluble Flkl/KDR receptor to treat a subjects having disease or disorder associated with VEGF. Active ingredients in such compositions may be present in free form or in the form of a pharmaceutical acceptable salt and optionally at least one pharmaceutically acceptable carrier.

The compounds described herein also may be used in combination with a second active ingredient which decreases the activity of, binds to, or inhibits the epidermal growth factor (EGF). Active ingredients in such compositions may be present in free form or in the form of a pharmaceutical acceptable salt and optionally at least one pharmaceutically acceptable carrier.

The compounds described herein also may be used to inhibit VEGF-mediated angiogenesis in a tissue via several methods including but not limited to, contacting the tissue with an inhibitor of NADPH oxidase and an effective amount of a compound of formula 1, by contacting the tissue with an inhibitor of reactive oxygen species (ROS) and an effective amount of a compound of Formula (I), or by contacting the tissue with an inhibitor of superoxide dismutase (SOD) and an effective amount of a compound of formula 1. Active ingredients in such compositions may be present in free form or in the form of a pharmaceutical acceptable salt and optionally at least one pharmaceutically acceptable carrier.

The compounds described herein may also be used in combination with molecules which specifically bind to placenta growth factor in order to suppress or prevent placenta growth factor-induced pathological angiogenesis, vascular leakage (oedema), pulmonary hypertension, tumour formation and/or inflammatory disorders.

The compounds described herein also may be used in combination with molecules chosen from the group comprising: an antibody or any fragment thereof which specifically binds to placenta growth factor, a small molecule specifically binding to placenta growth factor or to vascular endothelial growth factor receptor-1, -vascular endothelial growth factor receptor-1 antagonists or any fragment thereof, -a ribozyme against nucleic acids encoding placenta growth factor or the vascular endothelial growth factor receptor-1, and -anti-sense nucleic acids hybridizing with nucleic acids encoding placenta growth factor or vascular endothelial growth factor receptor-1. Active ingredients in such compositions may be present in free form or in the form of a pharmaceutical acceptable salt and optionally at least one pharmaceutically acceptable carrier.

The compounds described herein may be used in a method of inhibiting the growth of non-solid tumor cells that are stimulated by a ligand of vascular endothelial growth factor receptor (including but not limited to VEGFR2 kinase) in mammals, the method comprising treating the mammals with an effective amount of a compound of Formula (I). The compounds described herein may be used in a method of inhibiting the growth of non-solid tumors that are stimulated by a ligand of vascular endothelial growth factor receptor (including but not limited to VEGFR2 kinase) in mammals, the method comprising treating the mammals with an effective amount of a compound of Formula (I) in combination with radiation.

The compounds described herein may also be used in combination with G2/M agents and with therapeutic agents whose therapeutic effectiveness is dependent, at least in part, on the presence of an internalizing cell surface structure on the target cell. Such G2/M agents include but are not limited to vinorelbine tartrate, cisplatin, carboplatin, paclitaxel, doxorubicin, 5FU, docetaxel, vinblastine, vincristine, cyclophosphamide, apigenin, genistein, cycloxazoline. The compounds described herein may also be used in combination with substances which inhibit signal transduction mediated by human VEGF receptor Flt-1.

The compounds described herein may also be used for treating or preventing a tumor necrosis factor-mediated disease comprising co-administering a tumor necrosis factor alpha antagonist and an effective amount of a compound of Formula (I) to a patient. Contemplated tumor necrosis factor-mediated diseases include but are not limited to autoimmune disease, acute or chronic immune disease, inflammatory disease and neurodegenerative disease.

This invention further relates to a method for inhibiting abnormal cell growth in a mammal which method comprises administering to the mammal an amount of a compound of formula (I), or prodrugs thereof, pharmaceutically active metabolites, pharmaceutically acceptable salts, or pharmaceutically acceptable solvates of said compounds and said prodrugs, in combination with radiation therapy, wherein the amount of the compound, salt, solvate or prodrug is in combination with the radiation therapy effective in inhibiting abnormal cell growth in the mammal. Techniques for administering radiation therapy are known in the art, and these techniques can be used in the combination therapy described herein. The administration of the compound of the invention in this combination therapy can be determined as described herein.

It is believed that the compounds of formula (I) can render abnormal cells more sensitive to treatment with radiation for purposes of killing and/or inhibiting the growth of such cells. Accordingly, this invention further relates to a method for sensitizing abnormal cells in a mammal to treatment with radiation which comprises administering to the mammal an amount of a compound of formula (I), or prodrugs thereof, pharmaceutically active metabolites, pharmaceutically acceptable salts, or pharmaceutically acceptable solvates of said compounds and said prodrugs, which amount is effective in sensitizing abnormal cells to or enhancing the effects of treatment with radiation. The amount of the compound, salt, solvate or prodrug of formula (I) in this method can be determined according to the means for ascertaining effective amounts of such compounds described herein.

This invention further relates to a method for treating a disease related to vasculogenesis or angiogenesis in a mammal comprising administering to said mammal a therapeutically effective amount of a compound of formula (I), or prodrugs thereof, pharmaceutically active metabolites, pharmaceutically acceptable salts, or pharmaceutically acceptable solvates of said compounds and said prodrugs, in conjunction with a therapeutically effective amount of an anti-hypertensive agent.

Compounds of the present invention may be used in combination with CHK-1 inhibitors. Certain CHK-1 inhibitors have been proposed for cancer therapy (see Sanchez, Y., et. al. (1997) Science 277: 1497-1501 and Flaggs, G., et. al. (1997) Current Biology 7:977-986; U.S. Pat. Nos. 6,413,755, 6,383,744, and 6,211,164; and International Publication Nos. WO 01/16306, WO 01/21771, WO 00/16781, and WO 02/070494). In this embodiment, the CHK-1 inhibitor may be administered as a single agent or as co-therapy with other anti-neoplasm therapies including anti-neoplastic agents and radiation therapy.

The wide variety of available anti-neoplastic agents are contemplated for combination therapy with CHK-1 in accordance with present invention. In a preferred embodiment, anti-neoplastic agents that assert their cytotoxic effects by activating programmed cell death or apoptosis may be used in combination with the CHK-1 inhibitor. The anti-neoplastic agents contemplated in accordance with the present invention include, but are not limited to alkylating agents, including busulfan, chlorambucil, cyclophosphamide, iphosphamide, melphalan, nitrogen mustard, streptozocin, thiotepa, uracil nitrogen mustard, triethylenemelamine, temozolomide, and SARCnu; antibiotics and plant alkaloids including actinomycin-D, bleomycin, cryptophycins, daunorubicin, doxorubicin, idarubicin, irinotecan, L-asparaginase, mitomycin-C, mitramycin, navelbine, paclitaxel, docetaxel, topotecan, vinblastine, vincristine, VM-26, and VP-16-213; hormones and steroids including 5α-reductase inhibitor, aminoglutethimide, anastrozole, bicalutamide, chlorotrianisene, DES, dromostanolone, estramustine, ethinyl estradiol, flutamide, fluoxymesterone, goserelin, hydroxyprogesterone, letrozole, leuprolide, medroxyprogesterone acetate, megestrol acetate, methyl prednisolone, methyltestosterone, mitotane, nilutamide, prednisolone, SERM3, tamoxifen, testolactone, testosterone, triamicnolone, and zoladex; synthetics including all-trans retinoic acid, BCNU (carmustine), CBDCA carboplatin (paraplatin), CCNU (lomustine), cis-diaminedichloroplatinum (cisplatin), dacarbazine, gliadel, hexamethylmelamine, hydroxyurea, levamisole, mitoxantrone, o, p′-DDD (lysodren, mitotane), oxaliplatin, porfimer sodium, procarbazine, GleeVec; antimetabolites including chlorodeoxyadenosine, cytosine arabinoside, 2′-deoxycoformycin, fludarabine phosphate, 5-fluorouracil, 5-FUDR, gemcitabine, camptothecin, 6-mercaptopurine, methotrexate, MTA, and thioguanine; and biologics including alpha interferon, BCG, G-CSF, GM-CSF, interleukin-2, herceptin; and the like.

In a preferred embodiment of the invention, the anti-neoplastic agent is selected from the group consisting of alkylating agents, antibiotics and plant alkaloids, hormones and steroids, synthetic agents having anti-neoplastic activity, antimetabolites and biological molecules having anti-neoplastic activity.

In a preferred embodiment of the invention the antineoplastic agent is selected from the group consisting of Ara-c, VP-16, cis-platin, adriamycin, 2-chloro-2-deoxyadenosine, 9-β-D-arabinosyl-2-fluoroadenine, carboplatin, gemcitabine, camptothecin, paclitaxel, BCNU, 5-fluorouracil, irinotecan, and doxorubicin; more preferably gemcitabine.

The CHK-1 inhibitor in combination with the VEGF inhibitor identified in the present invention may also enhance the antineoplasm effects of radiation therapy. Usually, radiation can be used to treat the site of a solid tumor directly or administered by brachytherapy implants. The various types of therapeutic radiation which are contemplated for combination therapy in accordance with the present invention may be those used in the treatment of cancer which include, but are not limited to X-rays, gamma radiation, high energy electrons and High LET (Linear Energy Transfer) radiation such as protons, neutrons, and alpha particles. The ionizing radiation may be employed by techniques well known to those skilled in the art. For example, X-rays and gamma rays are applied by external and/or interstitial means from linear accelerators or radioactive sources. High-energy electrons may be produced by linear accelerators. High LET radiation is also applied from radioactive sources implanted interstitially.

The compounds of formula (I) or prodrugs thereof, pharmaceutically active metabolites, pharmaceutically acceptable salts, or pharmaceutically acceptable solvates of said compounds and said prodrugs, can each independently also be used in a palliative neo-adjuvant/adjuvant therapy in alleviating the symptoms associated with the diseases recited herein as well as the symptoms associated with abnormal cell growth. Such therapy can be a monotherapy or can be in a combination with chemotherapy and/or immunotherapy.

If the substituents themselves are not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999), which is incorporated herein by reference in its entirety. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

The compounds of the present invention may have asymmetric carbon atoms. Such diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of present invention may in certain instances exist as tautomers. This invention relates to the use of all such tautomers and mixtures thereof.

Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the formulae are intended to cover solvated as well as unsolvated forms of the identified structures. For example, Formula I includes compounds of the indicated structure in both hydrated and non-hydrated forms. Additional examples of solvates include the structures in combination with isopropanol, ethanol, methanol, DMSO, ethyl acetate, acetic acid, or ethanolamine.

In the case of agents that are solids, it is understood by those skilled in the art that the inventive compounds and salts may exist in different crystal or polymorphic forms, all of which are intended to be within the scope of the present invention and specified formulas.

This invention also encompasses pharmaceutical compositions containing and methods of treating bacterial infections through administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Definitions

As used herein, the following terms have the following meanings, unless expressly indicated otherwise.

The term “comprising” and “including” are used in their open, non-limiting sense.

The terms “abnormal cell growth” and “hyperproliferative disorder” are used interchangeably in this application.

“Abnormal cell growth” refers to cell growth that is independent of normal regulatory mechanisms (e.g., loss of contact inhibition), including the abnormal growth of normal cells and the growth of abnormal cells. This includes, but is not limited to, the abnormal growth of: (1) tumor cells (tumors), both benign and malignant, expressing an activated Ras oncogene; (2) tumor cells, both benign and malignant, in which the Ras protein is activated as a result of oncogenic mutation in another gene; (3) benign and malignant cells of other proliferative diseases in which aberrant Ras activation occurs. Examples of such benign proliferative diseases are psoriasis, benign prostatic hypertrophy, human papilloma virus (HPV), and restinosis. “Abnormal cell growth” also refers to and includes the abnormal growth of cells, both benign and malignant, resulting from activity of the enzyme farnesyl protein transferase.

The term “acyl” includes alkyl, aryl, or heteroaryl substituents attached to a compound via a carbonyl functionality (e.g., —C(O)-alkyl, —C(O)-aryl, etc.).

The term “acylamino” refers to an acyl radical appended to an amino or alkylamino group, and includes —C(O)—NH₂ and —C(O)—NRR″ groups where R and R′ are as defined in conjunction with alkylamino.

The term “acyloxy” refers to the ester group —OC(O)—R, where R is H, alkyl, alkenyl, alkynyl, or aryl. 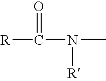
where each of R and R′ are independently selected from the group consisting of H, alkyl, and aryl.

The term “alkenyl” includes alkyl moieties having at least one carbon-carbon double bond, including E and Z isomers of said alkenyl moiety. The term also includes cycloalkyl moieties having at least one carbon-carbon double bond, i.e., cycloalkenyl. Examples of alkenyl radicals include ethenyl, propenyl, butenyl, 1,4-butadienyl, cyclopentenyl, cyclohexenyl, prop-2-enyl, but-2-enyl, but-3-enyl, 2-methylprop-2-enyl, hex-2-enyl, and the like. An alkenyl group may be optionally substituted.

The term “alkenylene” refers to a divalent straight chain, branched chain or cyclic saturated aliphatic group containing at least one carbon-carbon double bond, and including E and Z isomers of said alkenylene moiety. An alkyenylene group may be optionally substituted.

The term “alkoxyl” means an O-alkyl group. Examples of alkoxyl radicals include methoxyl, ethoxyl, n-propoxyl, isopropoxyl, n-butoxyl, iso-butoxyl, sec-butoxyl, tert-butoxyl and the like.

The term “alkyl” means saturated monovalent hydrocarbon radicals having straight, cyclic or branched moieties. An “alkyl” group may include an optional carbon-carbon double or triple bond where the alkyl group comprises at least two carbon atoms. Cycloalkyl moieties require at least three carbon atoms. Examples of straight or branched alkyl radicals include methyl (Me), ethyl (Et), n-propyl, isopropyl, n-butyl, isobutyl, sec-butyl, tert-butyl, tert-amyl, pentyl, isopentyl, hexyl, heptyl, octyl and the like. An alkyl group may be optionally substituted.

The term “alkylamino” refers to the —NRR′ group, where R and R′ are independently selected from hydrogen (however, R and R′ cannot both be hydrogen), alkyl, and aryl groups; or R and R′, taken together, can form a cyclic ring system.

The term “alkylene” refers to a divalent straight chain, branched chain or cyclic saturated aliphatic group. The latter group may also be referred to more specifically as a cycloalkylene group. An alkylene group may be optionally substituted.

The term “alkylthio” alone or in combination, refers to an optionally substituted alkyl thio radical, alkyl-S—.

The term “alkynyl” refers to straight- and branched-chain alkynyl groups having from two to twelve carbon atoms, preferably from 2 to 6 carbons, and more preferably from 2 to 4 carbons. Illustrative alkynyl groups include prop-2-ynyl, but-2-ynyl, but-3-ynyl, 2-methylbut-2-ynyl, hex-2-ynyl, and the like. An alkynyl group may be optionally substituted.

The term “amide” refers to the radical —C(O)N(R′)(R″) where R′ and R″ are each independently selected from hydrogen, alkyl, alkenyl, alkynyl, —OH, alkoxyl, cycloalkyl, heterocycloalkyl, heteroaryl, aryl as defined above; or R′ and R″ cyclize together with the nitrogen to form a heterocycloalkyl or heteroaryl.

The term “amino” refers to the —NH₂ group.

The term “anti-neoplastic agent” refers to agents capable of inhibiting or preventing the growth of neoplasms, or checking the maturation and proliferation of malignant (cancer) cells.

The term “aromatic” refers to compounds or moieties comprising multiple conjugated double bonds. Examples of aromatic moieties include, without limitation, aryl or heteroaryl ring systems.

The term “aryl” (Ar) means an organic radical derived from a monocyclic or polycyclic aromatic hydrocarbon by removal of one hydrogen, such as phenyl or naphthyl. Preferred aryl groups have from 4 to 20 ring atoms, and more preferably from 6 to 14 ring atoms. An aryl group may be optionally substituted. Illustrative examples of aryl groups include the following moieties: 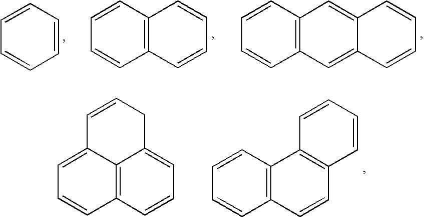
and the like.

The term “aryloxy” means aryl-O—.

The term “arylthio” means an aryl thio radical, aryl-S—.

The term “carbamoyl” or “carbamate” refers to the group —O—C(O)—NRR″ where R and R″ are independently selected from hydrogen, alkyl, and aryl groups; and R and R″ taken together can form a cyclic ring system.

The term “carbocyclyl” includes optionally substituted cycloalkyl and aryl moieties. The term “carbocyclyl” also includes cycloalkenyl moieties having at least one carbon-carbon double bond.

The term “carboxy esters” refers to —C(O)OR where R is alkyl or aryl.

The term “cycloalkyl” refers to a monocyclic or polycyclic radical which contains only carbon and hydrogen, and may be saturated, partially unsaturated, or fully unsaturated. A cycloalkyl group may be optionally substituted. Preferred cycloalkyl groups include groups having from three to twelve ring atoms, more preferably from 5 to 10 ring atoms. Illustrative examples of cycloalkyl groups include the following moieties: 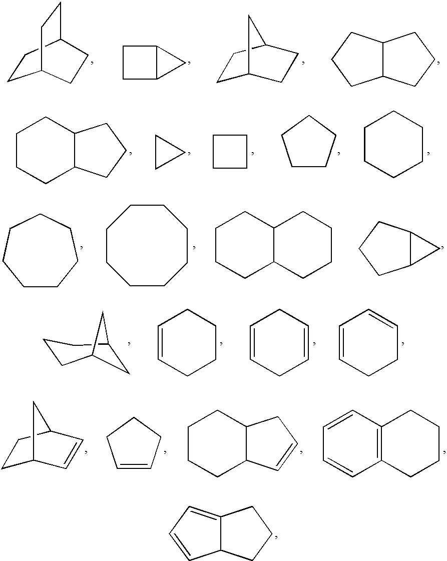
and the like.

The term “halo” or “halogen” means fluoro, chloro, bromo or iodo. Preferred halo groups are fluoro, chloro and bromo.

The terms haloalkyl, haloalkenyl, haloalkynyl and haloalkoxyl include alkyl, alkenyl, alkynyl and alkoxyl structures, that are substituted with one or more halo groups or with combinations thereof. The terms fluoroalkyl and fluoroalkoxyl include haloalkyl and haloalkoxyl groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other that carbon, e.g., oxygen, nitrogen, sulfur, phosphorus or combinations thereof.

The term “heteroaryl” (heteroAr) refers to an aryl group that includes one or more ring heteroatoms selected from nitrogen, oxygen and sulfur. A heteroaryl group may be optionally substituted. The polycyclic heteroaryl group may be fused or non-fused. Illustrative examples of aryl groups include the following moieties: 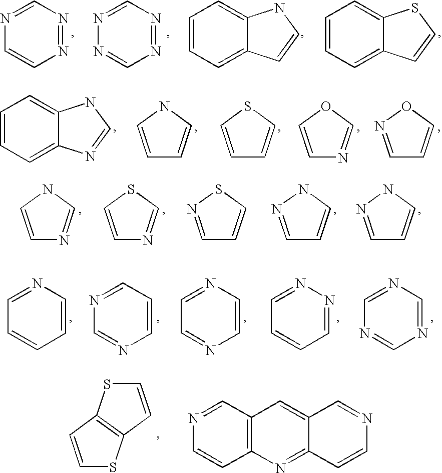
and the like.

The term “heterocyclyl” refers to aromatic and non-aromatic heterocyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl. An example of a 6 membered heterocyclic group is pyridyl, and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. A heterocyclyl group may be optionally substituted.

The term “heterocyclic” comprises both heterocycloalkyl and heteroaryl groups.

A “heterocycloalkyl” group refers to a cycloalkyl group that includes at least one heteroatom selected from nitrogen, oxygen and sulfur. The radicals may be fused with an aryl or heteroaryl. Illustrative examples of heterocycloalkyl groups include 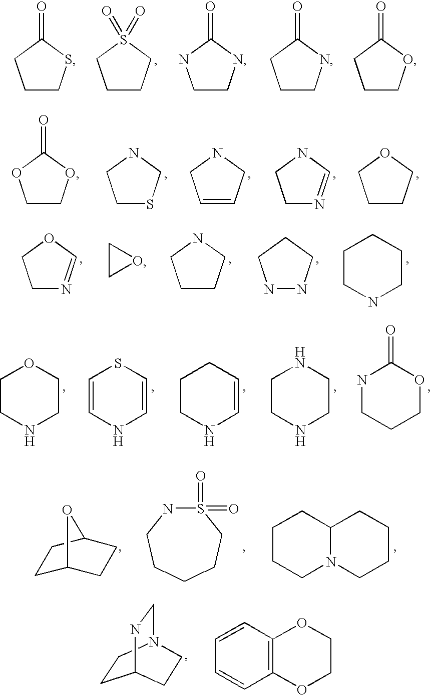
and the like.

The terms “5 membered heterocyclyl”, “5 or 6 membered heterocyclyl”, “5 to 8 membered heterocyclyl”, “5 to 10 membered heterocyclyl” or “5 to 13 membered heterocyclyl” includes aromatic and non-aromatic heterocyclyl groups containing one to four heteroatoms each selected from O, S and N, wherein each heterocyclyl group has from 5, 6, 5 to 8, 5 to 10 or 5 to 13 atoms in its ring system, respectively.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “neoplasm” is defined as in Stedman's Medical Dictionary 25^{th} Edition (1990) and refers to an abnormal tissue that grows by cellular proliferation more rapidly than normal and continues to grow after the stimuli that initiated the new growth ceases. Neoplasms show partial or complete lack of structural organization and functional coordination compared with normal tissue, and usually form a distinct mass of tissue that may be either benign (benign tumor) or malignant (cancer).

“Optionally substituted” groups may be substituted or unsubstituted. When substituted, the substituents of an “optionally substituted” group may include, without limitation, one or more substituents independently selected from the following groups or designated subsets thereof: (C₁-C₆)alkyl, (C₂-C₆)alkenyl, (C₂-C₆)alkynyl, (C₁-C₆)heteroalkyl, (C₁-C₆)haloalkyl, (C₂-C₆)haloalkenyl, (C₂-C₆)haloalkynyl, (C₃-C₆)cycloalkyl, phenyl, (C₁-C₆)alkoxyl, phenoxy, (C₁-C₆)haloalkoxyl, amino, (C₁-C₆)alkylamino, (C₁-C₆)alkylthio, phenyl-S—, oxo, (C₁-C₆)carboxyester, (C₁-C₆)carboxamido, (C₁-C₆)acyloxy, H, halogen, CN, NO₂, NH₂, N₃, NHCH₃, N(CH₃)₂, SH, SCH₃, OH, OCH₃, OCF₃, CH₃, CF₃, C(O)CH₃, CO₂CH₃, CO₂H, C(O)NH₂, pyridinyl, thiophene, furanyl, (C₁-C₆)carbamate, and (C₁-C₆)urea. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The term “oxo” means an “O” group.

The term “perhalo” refers to groups wherein every C—H bond has been replaced with a C-halo bond on an aliphatic or aryl group. Examples of perhaloalkyl groups include —CF₃ and —CFCl₂.

The term “substituted” means that the group in question, e.g., alkyl group, etc., may bear one or more substituents.

The term “ureyl” or “urea” refers to the group —N(R)—C(O)—NR′R″ where R, R′, and R″ are independently selected from hydrogen, alkyl, aryl; and where each of R—R′, R′—R″, or R—R″ taken together can form a cyclic ring system.

Pharmaceutical Formulations and Compositions

In addition to compounds of Formula I, the invention includes N-oxides, pharmaceutically acceptable prodrugs, pharmaceutically acceptable solvates, pharmaceutically active metabolites, and pharmaceutically acceptable salts of such compounds, prodrugs, solvates and metabolites.

The term “pharmaceutically acceptable” means pharmacologically acceptable and substantially non-toxic to the subject being administered the agent.

A “pharmacological composition” refers to a mixture of one or more of the compounds described herein, or physiologically acceptable salts thereof, with other chemical components, such as physiologically acceptable carriers and/or excipients. The purpose of a pharmacological composition is to facilitate administration of a compound to an organism.

A “physiologically acceptable carrier” refers to a carrier or diluent that does not cause significant or otherwise unacceptable irritation to an organism and does not unacceptably abrogate the biological activity and properties of the administered compound.

An “excipient” generally refers to substance, often an inert substance, added to a pharmacological composition or otherwise used as a vehicle to further facilitate administration of a compound. Examples of excipients include but are not limited to calcium carbonate, calcium phosphate, various sugars and types of starch, cellulose derivatives, gelatin, vegetable oils and polyethylene glycols.

The term “prodrug” means compounds that are drug precursors, which following administration, release the drug in vivo via some chemical or physiological process (e.g., a prodrug on being brought to the physiological pH is converted to the desired drug form).

Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula (I). The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

“A pharmaceutically acceptable prodrug” is a compound that may be converted under physiological conditions or by solvolysis to the specified compound or to a pharmaceutically acceptable salt of such compound. “A pharmaceutically active metabolite” is intended to mean a pharmacologically active product produced through metabolism in the body of a specified compound or salt thereof. Prodrugs and active metabolites of a compound may be identified using routine techniques known in the art. See, e.g., Bertolini, et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); and Larsen, Design and Application of Prodrugs, Drug Design and Development (Krogsgaard-Larsen et al., eds., Harwood Academic Publishers, 1991).

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, phosphoric acid and the like, or with an organic acid, such as acetic acid, phenylacetic acid, propionic acid, stearic acid, lactic acid, ascorbic acid, maleic acid, hydroxymaleic acid, isethionic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid, 2-acetoxybenzoic acid or cinnamic acid, a sulfonic acid, such as p-toluenesulfonic acid, methanesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine and piperazine, and inorganic salts derived from sodium, calcium, potassium, magnesium, manganese, iron, copper, zinc, aluminum and lithium.

Pharmaceutical compositions according to the invention may, alternatively or in addition to a compound of Formula (I), comprise as an active ingredient pharmaceutically acceptable prodrugs, pharmaceutically active metabolites, and pharmaceutically acceptable salts of such compounds and metabolites. Such compounds, prodrugs, multimers, salts, and metabolites are sometimes referred to herein collectively as “active agents” or “agents.”

It will be appreciated that any solvate (e.g. hydrate) form of compounds of formula (I) and prodrugs thereof can be used for the purpose of the present invention.

Therapeutically effective amounts of the active agents of the invention may be used to treat diseases mediated by modulation or regulation of protein kinases. An “effective amount” is intended to mean that amount of an agent that significantly inhibits proliferation and/or prevents de-differentiation of a eukaryotic cell, e.g., a mammalian, insect, plant or fungal cell, and is effective for the indicated utility, e.g., specific therapeutic treatment.

The compositions containing the compound(s) of the described herein can be administered for prophylactic and/or therapeutic treatments. In therapeutic applications, the compositions are administered to a patient already suffering from a proliferative disorder or condition (including, but not limited to, cancer), as described above, in an amount sufficient to cure or at least partially arrest the symptoms of the proliferative disorder or condition. An amount adequate to accomplish this is defined as “therapeutically effective amount or dose.” Amounts effective for this use will depend on the severity and course of the proliferative disorder or condition, previous therapy, the patient's health status and response to the drugs, and the judgment of the treating physician. In prophylactic applications, compositions containing the compounds described herein are administered to a patient susceptible to or otherwise at risk of a particular proliferative disorder or condition. Such an amount is defined to be a “prophylactically effective amount or dose.” In this use, the precise amounts also depend on the patient's state of health, weight, and the like. It is considered well within the skill of the art for one to determine such therapeutically effective or prophylactically effective amounts by routine experimentation (e.g., a dose escalation clinical trial).

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system (e.g., a tumor cell). An “enhancing-effective amount,” as used herein, refers to an amount adequate to enhance the effect of another therapeutic agent in a desired system (including, by way of example only, a tumor cell in a patient). When used in a patient, amounts effective for this use will depend on the severity and course of the proliferative disorder (including, but not limited to, cancer), previous therapy, the patient's health status and response to the drugs, and the judgment of the treating physician. It is considered well within the skill of the art for one to determine such enhancing-effective amounts by routine experimentation.

Once improvement of the patient's conditions has occurred, a maintenance dose is administered if necessary. Subsequently, the dosage or the frequency of administration, or both, can be reduced, as a function of the symptoms, to a level at which the improved proliferative disorder or condition is retained. When the symptoms have been alleviated to the desired level, treatment can cease. Patients can, however, require intermittent treatment on a long-term basis upon any recurrence of the disease symptoms.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. “Treating” is intended to mean at least the mitigation of a disease condition in a subject such as mammal (e.g., human), that is affected, at least in part, by the activity of one or more kinases, for example protein kinases such as tyrosine kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

Agents that potently regulate, modulate, or inhibit cell proliferation are preferred. For certain mechanisms, inhibition of the protein kinase activity associated with CDK complexes, among others, and those which inhibit angiogenesis and/or inflammation are preferred. The present invention is further directed to methods of modulating or inhibiting protein kinase activity, for example in mammalian tissue, by administering a compound of Formula (I). The activity of agents as anti-proliferatives is easily measured by known methods, for example by using whole cell cultures in an MTT assay. The activity of the compounds of Formula (I) as modulators of protein kinase activity, such as the activity of kinases, may be measured by any of the methods available to those skilled in the art, including in vivo and/or in vitro assays. Examples of suitable assays for activity measurements include those described in International Publication No. WO 99/21845; Parast et al., Biochemistry, 37,16788-16801 (1998); Connell-Crowley and Harpes, Cell Cycle: Materials and Methods, (Michele Pagano, ed. Springer, Berlin, Germany)(1995); International Publication No. WO 97/34876; and International Publication No. WO 96/14843. These properties may be assessed, for example, by using one or more of the biological testing procedures set out in the examples below.

The active agents of the invention may be formulated into pharmaceutical compositions as described below. Pharmaceutical compositions of this invention comprise an effective modulating, regulating, or inhibiting amount of a compound of Formula I and an inert, pharmaceutically acceptable carrier or diluent. In one embodiment of the pharmaceutical compositions, efficacious levels of the compounds of Formula (I) are provided so as to provide therapeutic benefits involving anti-proliferative ability. By “efficacious levels” is meant levels in which proliferation is inhibited, or controlled. These compositions are prepared in unit-dosage form appropriate for the mode of administration, e.g., parenteral or oral administration.

A compound of Formula (I) can be administered in conventional dosage form prepared by combining a therapeutically effective amount of an agent (e.g., a compound of Formula I) as an active ingredient with appropriate pharmaceutical carriers or diluents according to conventional procedures. These procedures may involve mixing, granulating and compressing or dissolving the ingredients as appropriate to the desired preparation.

The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a compound of Formula (I) can be dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease being treated. Optimal dosages for a given set of conditions can be ascertained by those skilled in the art using conventional dosage-determination tests in view of the experimental data for an agent. For oral administration, an exemplary daily dose generally employed is from about 0.001 to about 1000 mg/kg of body weight, with courses of treatment repeated at appropriate intervals. Administration of prodrugs is typically dosed at weight levels that are chemically equivalent to the weight levels of the fully active form.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations that can be used pharmaceutically.

Proper formulation is dependent upon the route of administration chosen. For injection, the agents of the invention may be formulated into aqueous solutions, preferably in physiologically compatible buffers such as Hanks's solution, Ringers solution, or physiological saline buffer. For transmucosal administration, penetrants appropriate to the barrier to be permeated are used in the formulation. Such penetrants are generally known in the art.

For oral administration, the compounds can be formulated readily by combining the compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of agents.

Pharmaceutical preparations that can be used orally include push-fit capsules made of gelatin, as well as soft, sealed capsules made of gelatin and a plasticizer, such as glycerol or sorbitol. The push-fit capsules can contain the agents in admixture with fillers such as lactose, binders such as starches, and/or lubricants such as talc or magnesium stearate, and, optionally, stabilizers. In soft capsules, the agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions take the form of tablets or lozenges formulated in conventional manners.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the agents in water-soluble form. Additionally, suspensions of the agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

For administration to the eye, the agent is delivered in a pharmaceutically acceptable ophthalmic vehicle such that the compound is maintained in contact with the ocular surface for a sufficient time period to allow the compound to penetrate the corneal and internal regions of the eye, for example, the anterior chamber, posterior chamber, vitreous body, aqueous humor, vitreous humor, cornea, iris/ciliary, lens, choroid/retina and sclera. The pharmaceutically acceptable ophthalmic vehicle may be an ointment, vegetable oil, or an encapsulating material. A compound of the invention may also be injected directly into the vitreous and aqueous humor.

Alternatively, the agents may be in powder form for constitution with a suitable vehicle, e.g., sterile pyrogen-free water, before use. The compounds may also be formulated in rectal compositions such as suppositories or retention enemas, e.g, containing conventional suppository bases such as cocoa butter or other glycerides.

In addition to the formulations described above, the agents may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semi-permeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

The agents of the invention may be useful in combination with known anti-cancer treatments such as: DNA interactive agents such as cisplatin or doxorubicin; topoisomerase 11 inhibitors such as etoposide; topoisomerase I inhibitors such as CPT-11 or topotecan; tubulin interacting agents such as paclitaxel, docetaxel or the epothilones; hormonal agents such as tamoxifen; thymidilate synthase inhibitors such as 5-fluorouracil; and anti-metalbolites such as methotrexate. They may be administered together or sequentially, and when administered sequentially, the agents may be administered either prior to or after administration of the known anticancer or cytotoxic agent.

The term “chemotherapeutic agent” as used herein includes, for example, hormonal agents, antimetabolites, DNA interactive agents, tubilin-interactive agents, and others such as aspariginase or hydroxyureas.

DNA-interactive agents include alkylating agents, such as cisplatin, cyclophosphamide, altretamine; DNA strand-breakage agents, such as bleomycin; intercalating topoisomerase II inhibitors, e.g., dactinomycin and doxorubicin); nonintercalating topoisomerase II inhibitors such as, etoposide and teniposide; and the DNA minor groove binder plicamydin, for example.

Alkylating agents may form covalent chemical adducts with cellular DNA, RNA, or protein molecules, or with smaller amino acids, glutathione, or similar chemicals. Examples of typical alkylating agents include, but are not limited to, nitrogen mustards, such as chlorambucil, cyclophosphamide, isofamide, mechlorethamine, melphalan, uracil mustard; aziridine such as thiotepa; methanesulfonate esters such as busulfan; nitroso ureas, such as carmustine, lomustine, streptozocin; platinum complexes, such as cisplatin, carboplatin; bioreductive alkylator, such as mitomycin, and procarbazine, dacarbazine and altretamine. DNA strand-breaking agents include bleomycin, for example.

DNA topoisomerase II inhibitors may include intercalators such as the following: amsacrine, dactinomycin, daunorubicin, doxorubicin (adriamycin), idarubicin, and mitoxantrone; as well as nonintercalators such as etoposide and teniposide.

An example of a DNA minor groove binder is plicamycin.

Antimetabolites generally interfere with the production of nucleic acids and thereby growth of cells by one of two major mechanisms. Certain drugs inhibit production of deoxyribonucleoside triphosphates that are the precursors for DNA synthesis, thus inhibiting DNA replication. Examples of these compounds are analogues of purines or pyrimidines and are incorporated in anabolic nucleotide pathways. These analogues are then substituted into DNA or RNA instead of their normal counterparts.

Antimetabolites useful as chemotherapeutic agents include, but are not limited to: folate antagonists such as methotrexate and trimetrexate; pyrimidine antagonists, such as fluorouracil, fluorodeoxyuridine, CB3717, azacitidine, cytarabine, and floxuridine; purine antagonists such as mercaptopurine, 6-thioguanine, fludarabine, pentostatin; and ribonucleotide reductase inhibitors such as hydroxyurea.

Tubulin interactive agents act by binding to specific sites on tubulin, a protein that polymerizes to form cellular microtubules. Microtubules are critical cell structure units and are required for cell division. These therapeutic agents disrupt the formation of microtubules. Exemplary tubulin-interactive agents include vincristine and vinblastine, both alkaloids and paclitaxel (Taxol).

Hormonal agents are also useful in the treatment of cancers and tumors, but only rarely in the case of B cell malignancies. They are used in hormonally susceptible tumors and are usually derived from natural sources. Hormonal agents include, but are not limited to, estrogens, conjugated estrogens and ethinyl estradiol and diethylstilbesterol, chlortrianisen and idenestrol; progestins such as hydroxyprogesterone caproate, medroxyprogesterone, and megestrol; and androgens such as testosterone, testosterone propionate; fluoxymesterone, and methyltestosterone.

Adrenal corticosteroids are derived from natural adrenal cortisol or hydrocortisone and are used to treat B cell malignancies. They are used because of their anti-inflammatory benefits as well as the ability of some to inhibit mitotic divisions and to halt DNA synthesis. These compounds include, but are not limited to, prednisone, dexamethasone, methylprednisolone, and prednisolone.

Leutinizing hormone releasing hormone agents or gonadotropin-releasing hormone antagonists are used primarily the treatment of prostate cancer. These include leuprolide acetate and goserelin acetate. They prevent the biosynthesis of steroids in the testes.

Antihormonal antigens include, for example, antiestrogenic agents such as tamoxifen, antiandrogen agents such as flutamide; and antiadrenal agents such as mitotane and aminoglutethimide.

Other agents include hydroxyurea (which appears to act primarily through inhibition of the enzyme ribonucleotide reductase), and asparaginase (an enzyme which converts asparagine to aspartic acid and thus inhibits protein synthesis).

Included within the scope of cancer therapy agents are radiolabeled antibodies, including but not limited to, Zevalin™ (IDEC Pharmaceuticals Corp.) and Bexxar™ (Corixa, Inc.); the use of any other radioisotope (e.g., ⁹⁰Y and ¹³¹I) coupled to an antibody or antibody fragment that recognizes an antigen expressed by a neoplasm; external beam radiation or any other method for administration of radiation to a patient.

Further included within the scope of cancer therapy agents are cytotoxins, including but not limited to an antibody or antibody fragment linked to a cytotoxin, or any other method for selectivly delivering a cytotoxic agent to a tumor cell.

Further included within the scope of cancer therapy agents are selective methods for destroying DNA, or any method for delivering heat to a tumor cells, including by way of example only, nanoparticles.

Further included within the scope of cancer therapy agents is the use of unlabeled antibodies or antibody fragments capable of killing or depleting tumor cells, including by way of example only, Rituxan™ (IDEC Pharmaceuticals Corp.) and Herceptin™ (Genentech).

The agents may be prepared using the reaction routes and synthesis schemes as described below, employing the general techniques known in the art using starting materials that are readily available. The preparation of preferred compounds of the present invention is described in detail in the following examples, but the artisan will recognize that the chemical reactions described may be readily adapted to prepare a number of other anti-proliferatives or protein kinase inhibitors of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### DETAILED DESCRIPTION OF THE INVENTION

The compounds of Formula (I) can act as antagonists of the VEGFR2. Without being bound to any particular theory, the linked rings are thought to provide favorable space-filling and electrostatic complementarity in the active site of the targeted protein.

In the examples described below, unless otherwise indicated, all temperatures are set forth in degrees Celsius and all parts and percentages are by weight. Reagents were purchased from commercial suppliers such as Aldrich Chemical Company or Lancaster Synthesis Ltd. and were used without further purification unless otherwise indicated. Tetrahydrofuran (THF), N,N-dimethylformamide (DMF), dichloromethane, toluene, and dioxane were purchased from Aldrich in Sure seal bottles and used as received. All solvents were purified using standard methods readily known to those skilled in the art, unless otherwise indicated.

The reactions set forth below were done generally under a positive pressure of argon or nitrogen or with a drying tube, at ambient temperature (unless otherwise stated), in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates Analtech (0.25 mm) and eluted with the appropriate solvent ratios (v/v), and are denoted where appropriate. The reactions were assayed by TLC and terminated as judged by the consumption of starting material.

Visualization of the TLC plates was done with a p-anisaldehyde spray reagent or phosphomolybdic acid reagent (Aldrich Chemical 20 wt % in ethanol) and activated with heat. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ prior to filtration and evaporation of the solvents under reduced pressure on a rotary evaporator and noted as solvents removed in vacuo. Flash column chromatography (Still, et al., J. Org. Chem., 43, 2923 (1978)) was done using Baker grade flash silica gel (47-61 μm) and a silica gel: crude material ratio of about 20:1 to 50:1 unless otherwise stated. Hydrogenolysis was done at the pressure indicated in the examples or at ambient pressure.

¹H-NMR spectra were recorded on a Bruker instrument operating at 300 MHz and ¹³C-NMR spectra were recorded operating at 75 MHz. NMR spectra were obtained as CDCl₃ solutions (reported in ppm), using chloroform as the reference standard (7.25 ppm and 77.00 ppm) or CD₃OD (3.4 and 4.8 ppm and 49.3 ppm), or internally tetramethylsilane (0.00 ppm) when appropriate. Other NMR solvents were used as needed. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

Infrared (IR) spectra were recorded on a Perkin-Elmer FT-IR Spectrometer as neat oils, as KBr pellets, or as CDCl₃ solutions, and when given are reported in wave numbers (cm⁻¹). The mass spectra were obtained using LSIMS or electrospray. All melting points (mp) are uncorrected.

### EXAMPLES

Preparative Methods:

The following methods describe typical synthetic procedures using specific materials. Many embodiments of the present invention may be synthesized using the described methods. The skilled artisan will recognize that different acids, amines, chloropyridyl derivatives, phenols and methyl ethers may be substituted in the following descriptions to suit the preparation of a desired embodiment. The following methods may be scaled upwards or downwards to suit the amount of desired material.

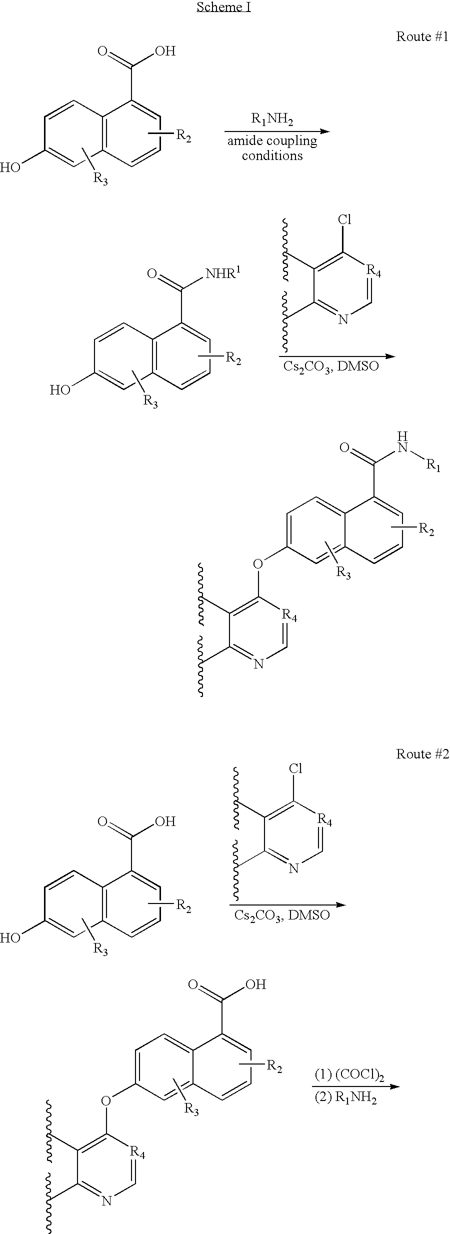

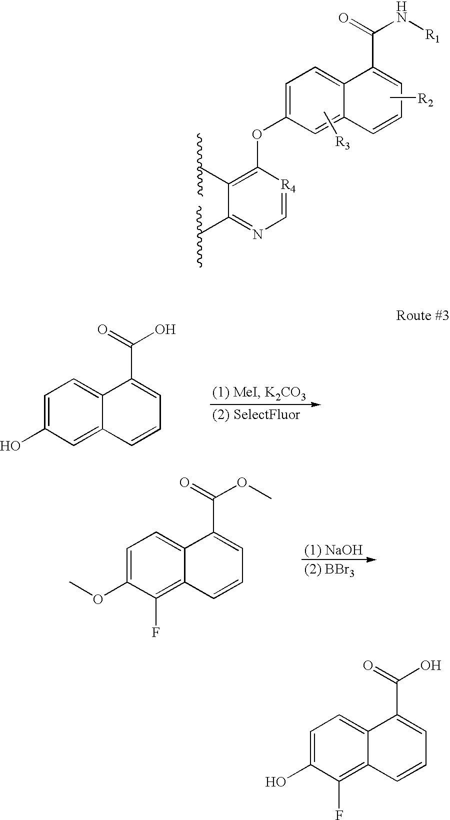

Scheme I describes and depicts two general routes (Routes #1 and #2) to prepare specific examples of the present invention. Scheme I also depicts a general route (route #3) for derivatizing naphthyl (or other aryl) moieties in the invention. In route #1, a naphthyl moiety is coupled to an amine to form an amide linkage. In second step, a derivatized chloropyridine moiety is coupled to a naphthylamide moiety via an ether linkage. In route #2 according to Scheme I, amide bond formation precedes ether bond formation

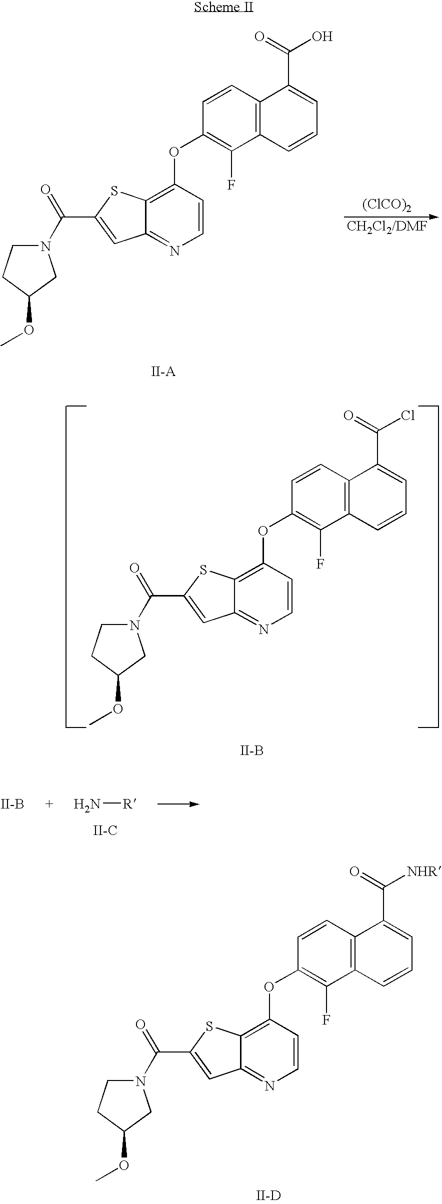

Method A for Amide Bond Formation: Method A, described and depicted in Scheme II, is a general method for amide bond formation beginning with a carboxylic acid and an amine. The carbonyl group of a carboxylic acid II-A is first activated by conversion to its corresponding carbonyl chloride II-B using oxalylchloride (or thionyl chloride). Carbonyl chloride II-B is then treated with amine II-C to yield desired amide II-D. The skilled artisan will recognize that many methods exist for the coupling of amines and carboxylates (for example Method B below, and the methods described herein are given by way of example.

Example of Method A: To a solution of acid, for example 5-fluoro-6-[(2{[(3S)-3-methoxypyrrolidin-1-yl]carbonyl}thieno[3,2-b]pyridin-7-yl)oxy]-1-naphthoic acid (0.090 g, 0.19 mmol) (see preparation of intermediate 1e in Example 1), in CH₂Cl₂ (5 mL) cooled at 0° C. was added oxalyl chloride (2.0 M in CH₂Cl₂, 0.290 mL, 0.58 mmol, 3 eq) or in certain cases neat SOCl₂, and DMF (2 drops). The reaction mixture was stirred at room temperature for 1 h, concentrated and dried under high vacuum. The residue was taken into CH₂Cl₂ or THF (5 mL), and amine, such as 2.0 M methylamine in THF (0.380 mL, 0.76 mmol, 4 eq) was added, and in some cases followed by addition of Et₃N (1˜1.5 eq). The reaction mixture was stirred at room temperature for 1 h, and then partitioned between H₂O (15 mL) and CH₂Cl₂ (2×15 mL). The combined organic layers were dried over MgSO₄ and concentrated. The residue was purified by flash column chromatography eluting with either gradients of EtOAc in hexanes or 0-10% CH₃OH in CH₂Cl₂ or by reverse phase HPLC to give corresponding amide in 20˜90% yield.

Example of Method B For Amide Bond Formation: To a solution of acid, for example 6-hydroxy-1-naphthoic acid (1.0 g, 5.31 mmol) in CH₂Cl₂ or DMF (10 mL) was added HATU (3.03 g, 7.97 mmol), amine, for example cyclopropylamine (0.442 mL, 6.37 mmol) and Et₃N (2.22 mL, 15.93 mmol). The reaction mixture was stirred at room temperature for 1 h, quenched with H₂O (60 mL) and extracted with CH₂Cl₂ (2×60 mL). The combined organic layers were dried over MgSO₄ and concentrated. The residue was purified by flash column chromatography, eluting with 1-10% CH₃OH in CH₂Cl₂, to give corresponding amide in 20-70% yield.

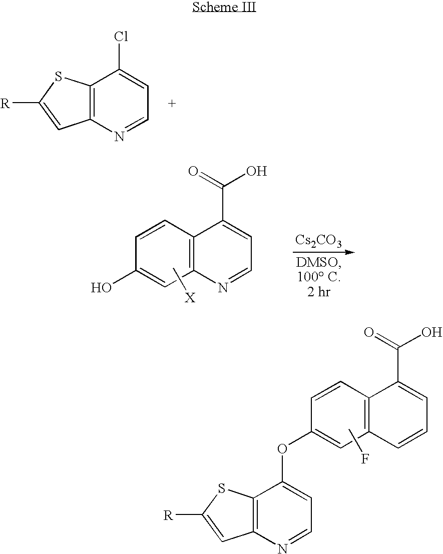

Method C for Coupling Chloropyridine Derivatives with Phenols: Method C follows the general procedure (route #2) provided in Scheme I. Method C is a general method for coupling chloropyridine moieties to naphthyl moieties via an ether linkage. In this method, chloride is displaced by naphthalate to yield an aryl naphthyl ether.

Example of Method C: Cs₂CO₃ (0.546 g, 1.68 mmol) was added to a solution of chloropyridine derivative, for example 7-chloro-2-{[(3S)-3-methoxypyrrolidin-1-yl]carbonyl}thieno[3,2-b]pyridine (0.239 g, 0.80 mmol), and phenol, such as 5-fluoro-6-hydroxy-1-naphthoic acid (0.138 g, 0.67 mmol) in DMSO. The reaction mixture was stirred at 120° C. for 2 h, and then quenched with H₂O (10 mL), washed with EtOAc (10 mL). The water layer was acidified with 1N HCl to pH˜7 or pH˜3 when the product contains carboxylic acid, filtered and the solid dried to give the desired ether which is either used without further purification or purified with reverse phase HPLC.

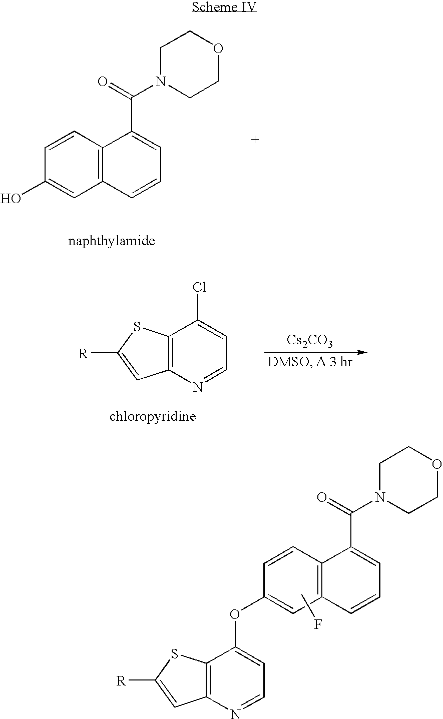

Method D for Coupling Chloropyridine Derivatives with Phenols: Method D also follows the general procedure provided in Scheme I (see Scheme I, route #1). Method D is a general method for coupling chloropyridine moieties to naphthyl moieties via an ether linkage. In this method, an amide moiety is already present in naphthylate which reacts with chloropyridine.

Example of Method D: To a stirred solution of phenol, such as 6-hydroxy-N-(2-morpholin-4-ylethyl)-1-naphthamide (12a) (106 mg, 0.35 mmol) and chloropyridyl derivative, such as (3R)-1-[(7-chlorothieno[3,2-b]pyridin-2-yl)carbonyl]pyrrolidin-3-ol (100 mg, 0.35 mmol) in DMA (5 mL) at ambient temperature was added Cs₂CO₃ (346 mg, 1.05 mmol), and the mixture was heated at 110° C. for 3 hours. The reaction was cooled to ambient temp, the solvent was removed in-vacuo and the dark residue was purified directly by SiO₂ chromatography (eluting with 95:5:0.5 CH₂Cl₂: MeOH: NH₃) to yield the title compound.

### EXAMPLES

### Example 1

### Preparation of 5-Fluoro-6-[(2-{[(3S)-3-methoxypyrrolidin-1-yl]carbonyl}thieno[3,2-b]pyridin-7-yl)oxy]-N-methyl-1-naphthamide

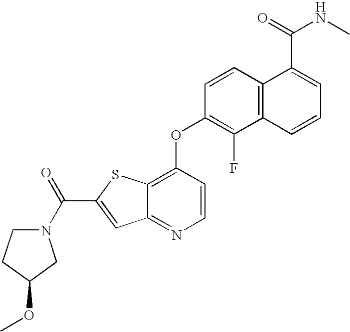

### A. Preparation of Intermediate 1a: Methyl 5-fluoro-6-methoxy-1-naphthoate

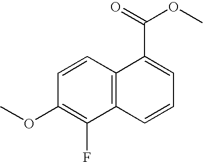

To a solution of methyl 6-methoxy-1-naphthoate (3.29 g, 15.4 mmol) in CH₃CN (10 mL) cooled at 0° C. was added SelectFluor fluorinating reagent (6.71 g, 18.9 mmol). The reaction mixture was stirred at room temperature overnight, quenched with H₂O (50 mL) and extracted with EtOAc (2×60 mL). The organic layers was dried over MgSO₄ and concentrated. The residue was purified by flash column chromatography eluting with 40% EtOAc in hexane to give an off-white solid (1.63 g, 51% yield). ¹H NMR (300 MHz, CDCl₃) δ8.70 (dd, 1H, J=9.51, 0.85 Hz), 8.24 (d, 1H, J=8.48 Hz), 8.09 (dd, 1H, J=7.25, 1.22 Hz), 7.51 (dd, 1H, J=8.48, 7.35 Hz), 7.40 (dd, 1H, J=9.33, 8.76 Hz), 4.04 (s, 3H), 3.99 (s, 3H).

### B. Preparation of Intermediate 1b: 5-Fluoro-6-methoxy-1-naphthoic Acid

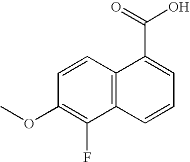

To a solution of methyl 5-fluoro-6-methoxy-1-naphthoate (1a) (1.63 g, 6.96 mmol) in MeOH (50 mL) was added NaOH aqueous solution (3N, 6.96 mL). The reaction mixture was stirred at reflux for 2 h, cooled to room temperature and concentrated. The residue was taken into H₂O (50 mL), acidified with 1N HCl, extracted with CH₂Cl₂ (2×60 mL), dried over MgSO₄ and concentrated to give a yellow solid (1.31 g, 86%). ¹H NMR (300 MHz, DMSO-d₆) δ13.23 (br. s, 1H), 8.69 (d, 1H, J=9.42 Hz), 8.19 (d, 1H, J=8.10 Hz), 8.07 (dd, 1H, J=7.16, 0.94 Hz), 7.66 (m, 2H), 4.00 (s, 3H).

### C. Preparation of Intermediate 1c: 5-Fluoro-6-hydroxy-1-naphthoic acid

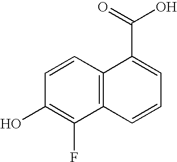

BBr₃ (1.0 M in CH₂Cl₂, 2.49 mL, 2.49 mmol) was added to a solution of 5-fluoro-6-methoxy-1-naphthoic acid (1b) (0.183 g, 0.83 mmol) in CH₂Cl₂ cooled at 0° C. The reaction mixture was stirred at 0° C. for 1 h, basified with saturated NH₄OH to pH 9, and stirred for 1 h. The mixture was acidified with 1N HCl to pH˜2, filtered and dried. The brown solid was used without further purification. ¹H NMR (300 MHz, DMSO-d₆) δ 10.25 (br. s, 1H), 8.52 (d, 1H, J=9.23 Hz), 8.12 (d, 1H, J=8.29 Hz), 7.98 (d, 1H, J=6.41 Hz), 7.58 (dd, 1H, J=8.19, 7.44 Hz), 7.37 (t, 1H, J=9.23 Hz), 6.98 (s, 1H). ESIMS (M+2Na⁺): 252.00.

### D. Preparation of Intermediate 1d: 7-Chloro-2-{[(3S)-3-methoxypyrrolidin-1-yl]carbonyl}thieno[3,2-b]pyridine

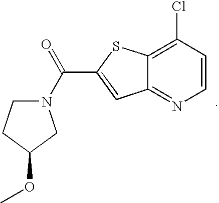

This material was prepared by the reaction of 7-chloro-thieno[3,2-b]pyridine-2-carboxylic acid lithium salt (prepared according to PCT application WO 01/94353, Example 1) (2.2 g, 10 mmol) and 3S-methoxy-pyrrolidine (1.11 g, 11 mmol) in a manner as described in Method A to give the desired amide as yellow oil (2.6 g, 80%). ¹H NMR (300 MHz, CDCl₃) δ 8.68 (d, 1H, J=5.5 Hz), 7.85 (d, 1H, J=14.3 Hz), 7.40 (d, 1H, J=5.5 Hz), 4.18-4.07 (m, 1H), 4.03-3.73 (m, 4H), 3.20 (d, 3H, J=14.5 Hz), 2.36-2.03 (m, 2H). LCMS ESI (M+H⁺): 297.05.

### E. Preparation of Intermediate 1e: 5-Fluoro-6-[(2-{[(3S)-3-methoxypyrrolidin-1-yl]carbonyl}thieno[3,2-b]pyridin-7-yl)oxy]-1-naphthoic acid

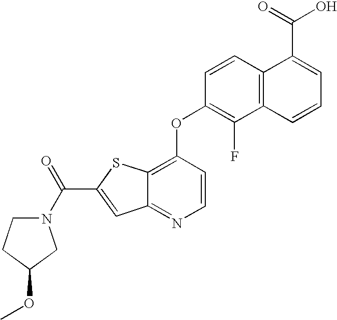

This material was prepared by the reaction of 7-chloro-2-{[(3S)-3-methoxypyrrolidin-1-yl]carbonyl}thieno[3,2-b]pyridine (1d) (0.239 g, 0.80 mmol), 5-fluoro-6-hydroxy-1-naphthoic acid (1c) (0.138 g, 0.67 mmol), and Cs₂CO₃ (0.546 g, 1.68 mmol) in a manner as described previously in Method C to give a brown solid (0.150 g, 48%). ¹H NMR (300 MHz, DMSO-d₆) δ8.84 (d, 1H, J=9.42 Hz), 8.59 (d, 1H, J=5.27 Hz), 8.35 (d, 1H, J=8.67 Hz), 8.29 (d, 1H, J=7.16 Hz), 8.10 (d, 1H, J=2.64 Hz), 7.78 (m, 1H), 6.85 (d, 1H, J=5.09 Hz), 3.97 (m, 4H), 3.62 (m, 3H), 3.25 (m, 2H), 2.02 (m, 2H).
F. Preparation of Title Compound

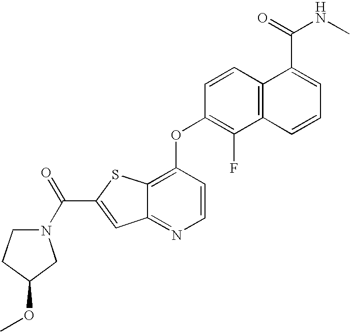

The compound of Example 1 was prepared by the coupling of 5-fluoro-6-[(2{[(3S)-3-methoxypyrrolidin-1-yl]carbonyl}thieno[3,2-b]pyridin-7-yl)oxy]-1-naphthoic acid (1 e) (0.090 g, 0.19 mmol) with methylamine (2.0 M in THF, 0.380 mL, 0.70 mL) in a manner as described in Method A to give an off-white solid (0.026 g, 29%). ¹H NMR (300 MHz, CDCl₃) δ8.47 (m, H), 8.22 (d, 1H, J=9.23 Hz), 8.14 (d, 1H, J=8.29 Hz), 7.87 (d, 1H, J=8.48 Hz), 7.66 (m, 1H), 7.54 (m, 1H), 7.40 (m, 1H), 6.58 (d, 1H, J=5.27 Hz), 6.51 (m, 1H), 4.07 (m, 1H), 3.92 (m, 2H), 3.79 (m, 1H), 3.70 (m, 1H), 3.35 (d, 3H, J=14.51 Hz), 3.08 (d, 3H, J=4.71 Hz), 2.24 (m, 1H), 2.12 (m, 1H). Anal. Calc'd. For C₂₅H₂₂FN₃O₄S.0.4CH₂Cl₂: C, 59.41; H, 4.48; N, 8.18; Found: C, 59.26; H, 4.60; N, 7.92. ESIMS (MH⁺): 480.05.

### Example 2

### Preparation of 7-({1-Fluoro-5-[(methylamino)carbonyl]-2-naphthyl}oxy)-N,N-dimethylthieno[3,2-b]pyridine-2-carboxamide

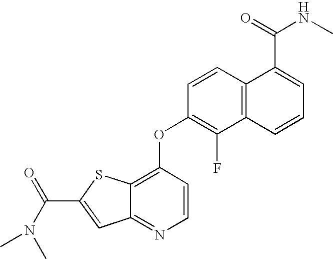

### A. Preparation of Intermediate 2a: 7-Chloro-N,N-dimethylthieno[3,2-b]pyridine-2-carboxamide

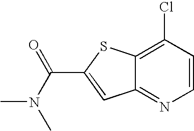

This material was prepared by the reaction of 7-chloro-thieno[3,2-b]pyridine-2-carboxylic acid (0.57 g, 2.67 mmol) with 2.0 M N,N-dimethylamine in THF (1.60 mL, 3.20 mmol) and Et₃N (0.447 mL, 3.20 mmol) in a manner as described in Method A to give the desired amide as brown solid (0.54 g, 84%). ¹H NMR (300 MHz, CDCl₃) δ 8.63(δ, 1H, J=4.85 Hz), 7.74 (s, 1H), 7.35 (d, 1H, J=5.02 Hz), 3.28 (s, 3H), 3.22 (s, 3H). ESIMS (MH⁺): 240.95.

### B. Preparation of Intermediate 2b: 6-({2-[(Dimethylamino)carbonyl]thieno[3,2-b]pyridin-7-yl}oxy)-5-fluoro-1-naphthoic Acid

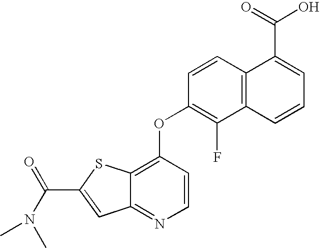

The material was prepared by the reaction of 7-chloro-N,N-dimethylthieno[3,2-b]pyridine-2-carboxamide (2a) (0.166 g, 0.69 mmol) with 5-fluoro-6-hydroxy-1-naphthoic acid (1c) (0.142 g, 0.69 mmol) and Cs₂CO₃ (0.526 g, 1.73 mmol) in a manner as described in Method C to give a brown solid (0.099 g, 35%). ESIMS (MH⁺): 411.00.

C. Preparation of Title Compound

The compound of Example 2 was prepared by the coupling of 6-({2-[(dimethylamino)carbonyl]thieno[3,2-b]pyridin-7-yl}oxy)-5-fluoro-1-naphthoic acid (2b) (0.063 g, 0.15 mmol) and methylamine (2.0M in THF, 0.385 mL, 0.77 mmol) in a manner as described previously in Method A to give a white solid (0.018 g, 28%). ¹H NMR (300 MHz, CDCl₃) δ8.51 (d, 1H, J=5.27 Hz), 8.21 (m, 2H), 7.71 (s, 1H), 7.67 (m, 1H), 7.58 (m, 1H), 7.43 (dd, 1H, J=9.04, 7.91 Hz), 6.56 (d, 1H, J=5.46 Hz), 6.20 (d, 1H, J=4.33 Hz), 3.29 (s, 3H), 3.18 (s, 3H)3.11 (d, 3H, J=4.90 Hz). ESIMS (MH⁺): 425.00.

### Example 3

### Preparation of 6-[(2-{[(3S)-3-methoxypyrrolidin-1-yl]carbonyl}thieno[3,2-b]pyridin-7-yl)oxy]-N-methyl-1-naphthamide

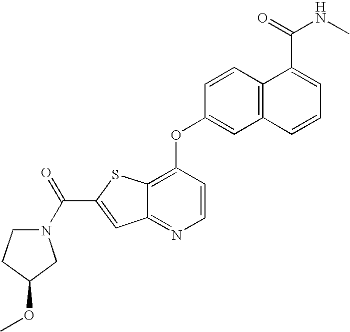

### A. Preparation of Intermediate 3a: 6-Hydroxy-N-methyl-1-naphthamide

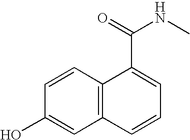

This material was prepared by the reaction of 6-hydroxy-1-naphthoic acid (1.0 g, 5.31 mmol) and 2.0 M methylamine in THF (10 mL, 20 mmol) in a manner as described previously in Method B to give a light yellow solid (0.48 g, 45% yield). ESIMS (M+H+): 202.0.

B. Preparation of Title Compound

The compound of Example 3 was prepared by the coupling of 7-chloro-2-{[(3S)-3-methoxypyrrolidin-1-yl]carbonyl}thieno[3,2-b]pyridine (1 d) with 6-hydroxy-N-methyl-1-naphthamide (3a) and Cs₂CO₃ in a manner as described previously in Method C to give an off-white solid. ¹H NMR (300 MHz, DMSO-d₆) δ8.60 (d, 1H, J=5.3 Hz), 8.54 (d, 1H, J=4.5 Hz), 8.35 (d, 1H, J=9.1 Hz), 8.08 (s, 1H), 8.01 (d, 1H, J=5.5 Hz), 7.91 (d, 1H, J=2.3 Hz), 7.61 (m, 2H), 7.54 (dd, 1H, J=9.3, 2.3 Hz), 6.84 (d, 1H, J=5.3 Hz), 4.03-3.73 (m, 3H), 3.60 (m, 2H), 3.25 (d, 3H, J=8.5 Hz), 2.85 (d, 3H, J=4.5 Hz), 2.04 (m, 2H). Anal. (C₂₅H₂₃N₃O₄S)C, H, N. ESIMS (MH+): 462.2.

### Example 4

### Preparation of N,N-Dimethyl-7-({5-[(methylamino)carbonyl]-2-naphthyl}oxy) thieno[3,2-b]pyridine-2-carboxamide

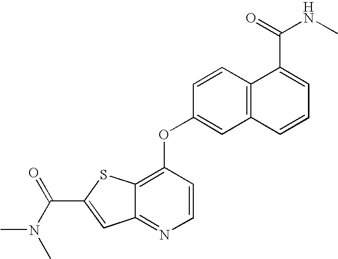

The compound of Example 4 was prepared by the reaction 7-chloro-N,N-dimethylthieno[3,2-b]pyridine-2-carboxamide (2a) (0.108 g, 0.45 mmol) with 6-hydroxy-N-methyl-1-naphthamide (3a) (0.090 g, 0.45 mmol) and Cs₂CO₃ (0.219 g, 0.67 mmol) in a manner as described previously in Method C to give a pale yellow solid (0.063 g, 35%). ¹H NMR (300 MHz, DMSO-d₆)δ8.59 (d, 1H, J=5.46 Hz), 8.53 (d, 1H, J=4.33 Hz), 8.36 (d, 1H, J=9.23 Hz), 8.02 (dd, 1H, J=7.54, 1.51 Hz), 7.95 (s, 1H), 7.91 (d, 1H, J-2.45 Hz), 7.61 (m, 2H), 7.54 (m, 1H), 6.83 (d, 1H, J=5.27 Hz), 3.33 (s, 3H), 3.26 (s, 3H), 2.85 (d, 3H, J=4.52 Hz). Anal. Calc'd. For C₂₂H₁₉N₃O₃S_{0.3}CH₃OH: C, 64.52; H, 4.91; N, 10.12; Found: C, 64.62; H, 4.92; N, 9.99. ESIMS (MH⁺): 406.10.

### Example 5

### Preparation of 6-[(2-{[(3R)-3-(dimethylamino)pyrrolidin-1-yl]carbonyl}thieno[3,2-b]pyridin-7-yl)oxy]-N-methyl-1-naphthamide

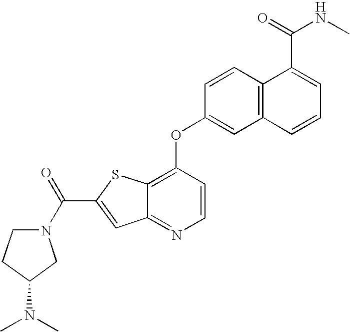

### A. Preparation of Intermediate 5a: (3R)-1-[(7-Chlorothieno[3,2-b]pyridin-2-yl)carbonyl]-N,N-dimethylpyrrolidin-3-amine

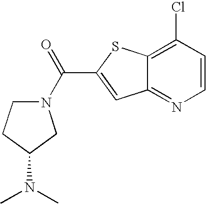

This material was prepared by the reaction of 7-chlorothieno[3,2-b]pyridine-2-carboxylic acid (0.214 g, 1.0 mmol) with (3R)-N,N-dimethylpyrrolidin-3-amine (0.1149, 1.0 mmol) and Et₃N (0.139 mL, 1.0 mmol) in a manner as described previously in Method A to give a brown solid (0.134 g, 43%). ¹H NMR (300 MHz, CD₃OD) δ7.24 (d, 1H, J=5.09 Hz), 6.57 (d, 1H, J=8.48 Hz), 6.15 (d, 1H, J=5.09 Hz), 2.70 (m, 1H), 2.51 (m, 2H), 2.24 (m, 1H), 2.04 (m, 1H), 1.49 (m, 1H), 0.93 (s, 3H), 0.90 (s, 3H), 0.52 (m, 1H). ESIMS (MH⁺): 310.10.

B. Preparation of Title Compound

The compound of Example 5 was prepared by the reaction of (3R)-1-[(7-chlorothieno[3,2-b]pyridin-2-yl)carbonyl]-N,N-dimethylpyrrolidin-3-amine (5a) (0.148 g, 0.48 mmol) with 6-hydroxy-N-methyl-1-naphthamide (3a) (0.096 g, 0.48 mmol) and Cs₂CO₃ (0.235 g, 0.72 mmol) in a manner as described previously in Method C to give a pale yellow solid (0.022 g, 10%). ¹H NMR (300 MHz, CD₃OD) δ 8.43 (d, 1H, J=5.46 Hz), 8.28 (d, 1H, J=9.23 Hz), 7.86 (m, 2H), 7.69 (d, 1H, J=2.07 Hz), 7.50 (m, 2H), 7.36 (dd, 1H, J=9.14, 2.17 Hz), 6.69 (d, 1H, J=5.27 Hz), 3.99 (m, 1H), 3.83 (m, 2H), 3.55 (m, 1H), 3.37 (m, 1H), 2.91 (s, 3H), 2.27 (s, 3H), 2.24 (s, 3H), 2.16 (m, 1H), 1.84 (m, 1H). Anal. Calc'd. For C₂₆H₂₆N₄O₃S.0.8H₂O: C, 61.60; H, 5.79; N, 10.38; Found: C, 61.13; H, 5.34; N, 10.85. ESIMS (MH⁺): 475.10.

### Example 6

### Preparation of N-[2-(Dimethylamino)ethyl]-N-methyl-7-({5-[(methylamino)carbonyl]-2-naphthyl}oxy)thieno[3,2-b]pyridine-2-carboxamide

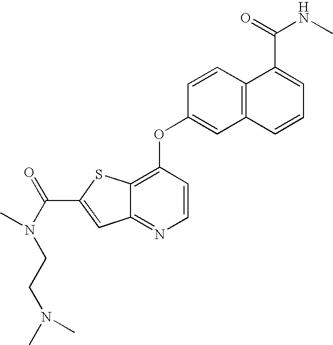

### A. Preparation of Intermediate 6a: 7-Chloro-N-[2-(dimethylamino)ethyl]-N-methylthieno[3,2-b]pyridine-2-carboxamide

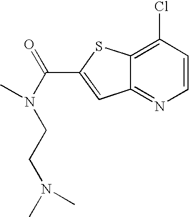

This material was prepared by the reaction of 7-chlorothieno[3,2-b]pyridine-2-carboxylic acid (0.957 g, 4.48 mmol) with N,N,N′-trimethylethane-1,2-diamine (0.640 mL, 4.93 mmol) and Et₃N (0.624 mL, 4.48 mmol) in a manner as described previously in Method A to give a brown solid (0.167 g, 13%). ¹H NMR (400 MHz, CDCl₃) δ8.60 (d, 1H, J=5.05 Hz), 7.74 (s, 1H), 7.32 (d, 1H, J=5.05 Hz), 3.66 (t, 2H, J=6.19 Hz), 3.26 (s, 3H), 2.57 (t, 2H, J=6.69 Hz), 2.25 (s, 6H). ESIMS (MH⁺): 298.05.

B. Preparation of Title Compound

The compound of Example 6 was prepared by the reaction 7-chloro-N-[2-(dimethylamino)ethyl]-N-methylthieno[3,2-b]pyridine-2-carboxamide (6a) (0.130 g, 0.44 mmol) with 6-hydroxy-N-methyl-1-naphthamide (3a) (0.088 g, 0.44 mmol) and Cs₂CO₃ (0.215 g, 0.66 mmol) in a manner as described previously in Method C to give a white solid (0.084 g, 41%). ¹H NMR (300 MHz, DMSO-d₆) δ8.53 (d, 1H, J=5.46 Hz), 8.47 (m, 1H), 8.30 (d, 1H, J=9.23 Hz), 7.96 (dd, 1H, J=7.35, 1.88 Hz), 7.87 (s, 1H), 7.85 (d, 1H, J=2.64 Hz), 7.56 (m, 2H), 7.48 (dd, 1H, J=9.23, 2.45 Hz), 6.78 (d, 1H, J=5.27 Hz), 3.54 (t, 2H, J=6.22 Hz), 3.26 (s, 3H), 2.98 (m, 2H), 2.79 (d, 3H, J=4.52 Hz), 2.10 (s, 6H). Anal. Calc'd. For C₂₅H₂₆N₄O₃S.0.7H₂O: C, 63.19; H, 5.81; N, 11.79; Found: C, 63.04; H, 5.46; N, 11.67. ESIMS (MH⁺): 463.15.

### Example 7

### Preparation of N-[3-(Dimethylamino)propyl]-N-methyl-7-({5-[(methylamino) carbonyl]-2-naphthyl}oxy)thieno[3,2-b]pyridine-2-carboxamide

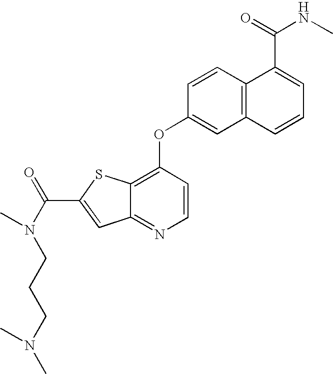

### A. Preparation of Intermediate 7a: 7-Chloro-N-[3-(dimethylamino)propyl]-N-methylthieno[3,2-b]pyridine-2-carboxamide

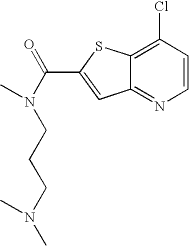

This material was prepared by the reaction of 7-chlorothieno[3,2-b]pyridine-2-carboxylic acid (1.0 g, 4.68 mmol) with N,N,N′-trimethylpropane-1,3-diamine (0.868 mL, 4.68 mmol) and Et₃N (1.96 mL, 14.04 mmol) in a manner as described previously in Method A to give a white foam (1.07 g, 77%). ¹H NMR (300 MHz, CD₃OD) δ 8.56 (d, 1H, J=5.09 Hz), 7.76 (s, 1H), 7.46 (d, 1H, J=5.27 Hz), 3.51 (m, 2H), 3.20 (s, 3H), 2.33 (m, 2H), 2.18 (s, 6H), 1.79 (m, 2H). ESIMS (MH⁺): 312.05.

B. Preparation of Title Compound

The compound of Example 7 was prepared by the reaction 7-chloro-N-[3-(dimethylamino)propyl]-N-methylthieno[3,2-b]pyridine-2-carboxamide (7a) (0.119 g, 0.38 mmol) with 6-hydroxy-N-methyl-1-naphthamide (3a) (0.077 g, 0.38 mmol) and Cs₂CO₃ (0.186 g, 0.57 mmol)) in a manner as described previously in Method C to give a white solid (0.038 g, 21%). ¹H NMR (300 MHz, DMSO-d₆) δ8.42 (d, 1H, J=5.46 Hz), 8.27 (d, 1H, J=9.23 Hz), 7.86 (d, 1H, J=7.91 Hz), 7.67 (m, 2H), 7.49 (m, 2H), 7.35 (dd, 1H, J=9.23, 2.45 Hz), 6.68 (d, 1H, J=5.46 Hz), 6.68 (d, 1H, J=5.46 Hz), 3.50 (m, 2H), 3.20 (m, 3H), 3.06 (m, 2H), 2.90 (s, 3H), 2.22 (s, 6H), 1.79 (m, 2H). Anal. Calc'd. For C₂₆H₂₈N₄O₃S.1.8H₂O: C, 61.35; H, 6.26; N, 11.01; Found: C, 60.94; H, 5.78; N, 11.12. ESIMS (MH⁺): 477.10.

### Example 8

### Preparation of N-(2-Hydroxyethyl)-N-methyl-7-({5-[(methylamino)carbonyl]-2-naphthyl}oxy)thieno[3,2-b]pyridine-2-carboxamide

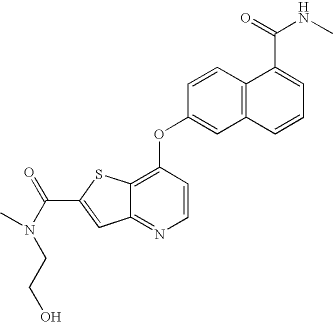

### A. Preparation of Intermediate 8a: 7-Chloro-N-(2-hydroxyethyl)-N-methylthieno[3,2-b]pyridine-2-carboxamide

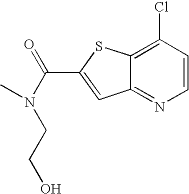

This material was prepared by the reaction of 7-chlorothieno[3,2-b]pyridine-2-carboxylic acid (1.0 g, 4.68 mmol) with 2-(methylamino)ethanol (0.414 mL, 5.15 mmol) and Et₃N (0.718 mL, 5.15 mmol) in a manner as described in Method A to give a pale brown color solid (0.624 g, 49% yield). ¹H NMR (400 MHz, CDCl₃) δ 8.61 (d, 1H, J=4.80 Hz), 7.80 (s, 1H), 7.33 (d, 1H, J=4.55 Hz), 3.92 (m, 2H), 3.76 (t, 2H, J=5.05 Hz), 3.37 (s, 3H), 3.19 (s, 1H). ESIMS (MH⁺): 259.10.

### B. Preparation of Intermediate 8b: N-(2-{[tert-Butyl(dimethyl)silyl]oxy} ethyl)-7-chloro-N-methylthieno[3,2-b]pyridine-2-carboxamide

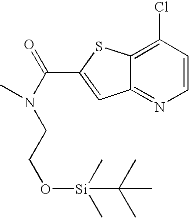

To a solution of 7-chloro-N-(2-hydroxyethyl)-N-methylthieno[3,2-b]pyridine-2-carboxamide (8a) (1.27 g, 4.68 mmol) in CH₂Cl₂ (25 mL) was added t-butyldimethylsilyl chloride (0.705 g, 4.68 mmol) and Et₃N (0.718 mL, 4.68 mmol). The reaction mixture was stirred at room temperature for 1 h and was partitioned between CH₂Cl₂ (50 mL) and H₂O (50 mL). The layers were separated and the aqueous phase was extracted with CH₂Cl₂ (50 mL). The combined organic layers were dried over MgSO₄ and concentrated. The residue was purified by flash column chromatography eluting with 25-30% EtOAc in hexane to give a light orange color oil (1.40 g, 78%). ¹H NMR (300 MHz, CDCl₃) δ 8.61 (d, 1H, J=5.09 Hz), 7.74 (s, 1H), 7.32 (d, 1H, J=5.09 Hz), 3.89 (m, 2H), 3.71 (m, 2H), 3.37 (s, 3H), 0.89 (m, 9H), 0.07 (m, 6H). ESIMS (MH⁺): 385.10.

C. Preparation of Title Compound

The compound of Example 8 was prepared by the reaction N-(2-{[tert-butyl(dimethyl)silyl]oxy}ethyl)-7-chloro-N-methylthieno[3,2-b]pyridine-2-carboxamide (8b) (0.171 g, 0.44 mmol) with 6-hydroxy-N-methyl-1-naphthamide (3a) (0.089 g, 0.44 mmol) and Cs₂CO₃ (0.215 g, 0.66 mmol)) in a manner as described previously in Method C to give a white solid (0.095 g, 5%). ¹H NMR (300 MHz, CD₃OD) δ 8.28 (d, 1H, J=9.23 Hz), 7.89 (d, 1H, J=8.29 Hz), 7.82 (m, 1H), 7.74 (m, 1H), 7.69 (d, 1H, J=2.07 Hz), 7.55 (m, 1H), 7.49 (d, 1H, J=7.54 Hz), 7.37 (dd, 1H, J=9.04, 2.26 Hz), 6.70 (d, 1H, J=5.09 Hz), 3.71 (m, 2H), 3.63 (d, 3H, J=4.52 Hz), 3.29 (m, 2H), 2.91 (s, 3H). Anal. Calc'd. For C₂₃H₂₁N₃O₄S 1.2H₂O.0.2EtOAc: C, 60.21; H, 5.31; N, 8.85; Found: C, 60.59; H, 4.91; N, 8.67. ESIMS (MH⁺): 436.15.

### Example 9

### Preparation of N-Cyclopropyl-6-[(2-{[(3R)-3-(dimethylamino)pyrrolidin-1-yl]carbonyl}thieno[3,2-b]pyridin-7-yl)oxy]-1-naphthamide

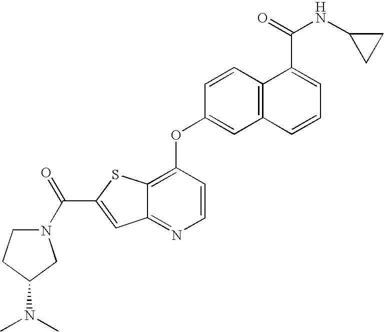

### A. Preparation of Intermediate 9a: N-Cyclopropyl-6-hydroxy-1-naphthamide

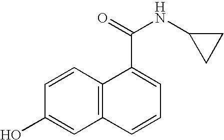

This material was prepared by the reaction of 6-hydroxy-1-naphthoic acid (1.0 g, 5.31 mmol) with cyclopropylamine (0.442 mL, 6.37 mmol) and Et₃N (2.22 mL, 15.93 mmol) in a manner as described previously in Method B to give an off-white solid (0.46 g, 38%); ¹H NMR (300 MHz, CD₃OD) δ 7.93 (d, 1H, J=9.98 Hz), 7.62 (d, 1H, J=8.10 Hz), 7.24 (m, 2H), 7.02 (dd, 2H, J=7.35, 2.45 Hz), 2.94 (m, 1H), 0.74 (m, 2H), 0.56 (m, 2H). ESIMS (MNa⁺): 250.15.

B. Preparation of Title Compound

The compound of Example 9 was prepared by the reaction of (3R)-1-[(7-chlorothieno[3,2-b]pyridin-2-yl)carbonyl]-N,N-dimethylpyrrolidin-3-amine (5a) (0.139 g, 0.45 mmol) with N-cyclopropyl-6-hydroxy-1-naphthamide (9a) (0.102 g, 0.45 mmol) and Cs₂CO₃ (0.220 g, 0.68 mmol)) in a manner as described previously in Method C to give a white solid (0.015 g, 7%). ¹H NMR (300 MHz, CD₃OD) □8.45 (d, 1H, J=5.46 Hz), 8.28 (d, 1H, J=9.23 Hz), 7.87 (m, 2H), 7.70 (d, 1H, J=2.45 Hz), 7.50 (m, 2H), 7.38 (dd, 1H, J=9.14, 2.54 Hz), 6.71 (d, 1H, J=5.46 Hz), 4.04 (m, 1H), 3.83 (m, 1H), 3.57 (m, 1H), 3.38 (m, 1H), 2.89 (m, 2H), 2.29 (s, 3H), 2.25 (s, 3H), 2.18 (m, 1H), 1.86 (m, 1H), 0.76 (m, 2H), 0.59 (m, 2H). Anal. Calc'd. For C₂₈H₂₈N₄O₃S.1.5EtOAc: C, 63.03; H, 5.80; N, 9.49; Found: C, 63.47; H, 5.28; N, 9.94. ESIMS (MH⁺): 501.10.

### Example 10

### Preparation of N-Methyl-6-{[2-({3-[(methylamino)methyl]pyrrolidin-1-yl}carbonyl)thieno[3,2-b]pyridin-7-yl]oxy}-1-naphthamide

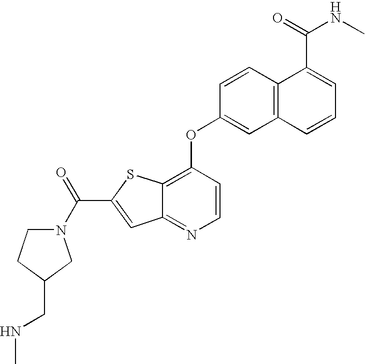

### A. Preparation of Intermediate 10a: tert-Butyl pyrrolidin-3-ylmethylcarbamate

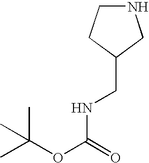

To a solution of tert-butyl (1-benzylpyrrolidin-3-yl)methylcarbamate (3.0 g, 10.33 mmol) in EtOAc (100 mL) was added Pd(OH)₂ on carbon (0.3 g). The mixture was stirred under H₂ balloon at room temperature for 3 h and filtered through Celite. The filtrate was concentrated to give colorless oil (1.87 g, 90%) and used as it is for the next step.

### B. Preparation of Intermediate 10b: tert-Butyl {1-[(7-chlorothieno[3,2-b]pyridin-2-yl)carbonyl]pyrrolidin-3-yl}methylcarbamate

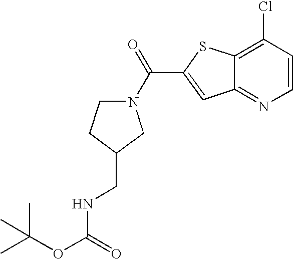

This material was prepared from the reaction of 7-chlorothieno[3,2-b]pyridine-2-carboxylic acid lithium salt (2.27 g, 10.33 mmol) with tert-butyl pyrrolidin-3-ylmethylcarbamate (10a) (2.07 g, 10.33 mmol) and Et₃N (1.44 mL, 10.33 mmol) in a manner as described previously in Method A to give a yellow solid (2.44 g, 60%). ¹H NMR (300 MHz, CDCl₃) δ 7.85 (s, 1H), 7.34 (d, 1H, J=5.09 Hz), 4.73 (s, 1H), 3.96 (m, 1H), 3.85 (m, 1H), 3.70 (m, 1H), 3.55 (m, 1H), 3.42 (m, 1H), 3.22 (m, 2H), 2.54 (m, 1H), 2.12 (m, 1H), 1.42 (d, 9H, J=8.29 Hz). ESIMS (M+): 396.05.

### C. Preparation of Intermediate 10c: N-({1-[(7-Chlorothieno[3,2-b]pyridin-2-yl)carbonyl]pyrrolidin-3-yl}methyl)-N-methylamine

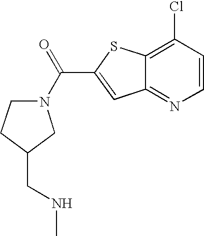

NaH (0.033 g, 0.82 mmol) and CH₃I (0.064 mL, 1.02 mmol) were added to a solution of tert-butyl {1-[(7-chlorothieno[3,2-b]pyridin-2-yl)carbonyl]pyrrolidin-3-yl}methyl-carbamate (10b) (0.271 g, 0.68 mmol) in THF at 0° C. The reaction mixture was stirred and warmed to room temperature overnight. TFA was added to the mixture and stirred for 2 hours at ambient temperature. The mixture was neutralized with aqueous NaHCO₃ and partitioned between H₂O (50 mL) and EtOAc (2×50 mL). The combined organic layers were dried over MgSO₄ and concentrated. The residue was purified by flash column chromatography eluting with 0-2% CH₃OH in CH₂Cl₂ to give a yellow solid (0.283 g, 82%). ¹H NMR (300 MHz, CDCl₃) □8.62 (d, 1H, J=5.09 Hz), 7.85 (s, 1H), 7.34 (d, 1H, J=5.09 Hz), 4.72 (s, 1H), 3.99 (m, 1H), 3.77 (m, 2H), 3.50 (m, 1H), 3.19 (m, 2H), 2.88 (d, 3H, J=12.06 Hz), 2.62 (m, 1H), 2.14 (m, 1H), 1.83 (m, 1H). ESIMS (MH⁺): 310.10.

D. Preparation of Title Compound

The compound of Example 10 was prepared by the reaction N-({1-[(7-chlorothieno[3,2-b]pyridin-2-yl)carbonyl]pyrrolidin-3-yl}methyl)-N-methylamine (10c) (0.098 g, 0.35 mmol) with 6-hydroxy-N-methyl-1-naphthamide (3a) (0.071 g, 0.35 mmol) and Cs₂CO₃ (0.171 g, 0.53 mmol)) in a manner as described previously in Method C to give a white solid (0.037 g, 22%). ¹H NMR (300 MHz, CD₃OD) δ 8.40 (d, 1H, J=4.71 Hz), 8.25 (d, 1H, J=9.04 Hz), 7.85 (d, 1H, J=8.10 Hz), 7.78 (s, 1H), 7.65 (m, 1H), 7.47 (m, 2H), 7.32 (d, 1H, J=9.04 Hz), 6.65 (d, 1H, J=5.46 Hz), 3.90 (m, 3H), 3.53 (m, 1H), 3.24 (m, 1H), 2.84 (m, 5H), 2.50 (d, 3H, J=10.36 Hz), 2.13 (m, 1H), 1.73 (m, 1H). Anal. Calc'd. For C₂₆H₂₆N₄O₃S.0.5H₂O.2.0AcOH: C, 59.68; H, 5.84; N, 9.28; Found: C, 59.50; H, 5.42; N, 9.52. ESIMS (MH⁺): 475.10.

### Example 11

### Preparation of 6-[(2-{[(3R)-3-(Dimethylamino)pyrrolidin-1-yl]carbonyl}thieno[3,2-b]pyridin-7-yl)oxy]-N-(2-methoxyethyl)-1-naphthamide

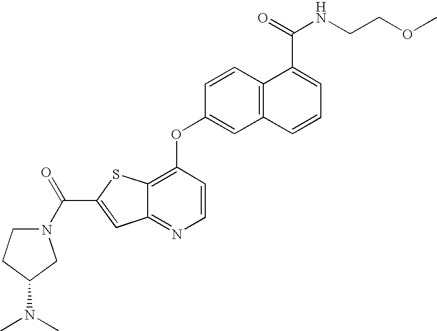

### A. Preparation of Intermediate 11a: 6-[(2-{[(3R)-3-(Dimethylamino)pyrrolidin-1-yl]carbonyl}thieno[3,2-b]pyridin-7-yl)oxy]-1-naphthoic Acid

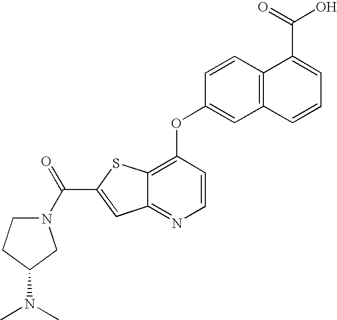

This material was prepared by the reaction of 6-hydroxy-1-naphthoic acid (0.080 g, 0.43 mmol) with (3R)-1-[(7-chlorothieno[3,2-b]pyridin-2-yl)carbonyl]-N,N-dimethylpyrrolidin-3-amine (5a) (0.132 g, 0.43 mmol) and Cs₂CO₃ (0.350 g, 1.08 mmol) in a manner as described previously in Method C to give a brown solid (0.118 g, 60%). ¹H NMR (300 MHz, DMSO-d₆)δ 8.98 (d, 1H, J=9.42 Hz), 8.57 (d, 1H, J=5.46 Hz), 8.13 (m, 2H), 8.03 (s, 1H), 7.91 (d, 1H, J=2.64 Hz), 7.58 (m, 2H), 6.83 (d, 1H, J=5.46 Hz), 4.03 (m, 1H), 3.78 (m, 2H), 3.42 (m, 1H), 3.11 (m, 1H), 2.40 (d, 6H, J=7.35 Hz), 2.21 (m, 1H), 1.86 (m, 1H).

B. Preparation of Title Compound

The compound of Example 11 was prepared by the reaction of 6-[(2-{[(3R)-3-(dimethylamino) pyrrolidin-1-yl]carbonyl}thieno[3,2-b]pyridin-7-yl)oxy]-1-naphthoic acid (11a) (0.118 g, 0.25 mmol) and 2-methoxyethylamine (0.043 mL, 0.5 mmol) in a manner as described previously in Method A to give an off-white solid (0.016 g, 12%). ¹H NMR (300 MHz, CDCl₃) δ 8.51 (d, 1H, J=5.27 Hz), 8.46 (d, 1H, J=9.23 Hz), 7.86 (m, 2H), 7.63 (m, 2H), 7.51 (d, 1H, J=8.10 Hz), 7.38 (dd, 1H, J=9.14, 2.36 Hz), 6.63 (d, 1H, J=5.46 Hz), 6.52 (t, 1H, J=5.46 Hz), 3.94 (m, 2H), 3.73 (m, 2H), 3.61 (m, 3H), 3.45 (m, 1H), 3.39 (s, 3H), 2.77 (m, 1H), 2.29 (d, J=8.29 Hz, 6H), 2.21 (m, 1H), 1.93 (m, 1H). Anal. Calc'd. For C₂₈H₃₀N₄O₄S.1.2H₂O: C, 62.25; H, 6.05; N, 10.37; Found: C, 62.15; H, 5.93; N, 10.22. ESIMS (MH⁺): 519.20.

### Example 12

### Preparation of 6-[(2-{[(3R)-3-(Dimethylamino)pyrrolidin-1-yl]carbonyl}thieno[3,2-b]pyridin-7-yl)oxy]-N-(2-morpholin-4-ylethyl)-1-naphthamide

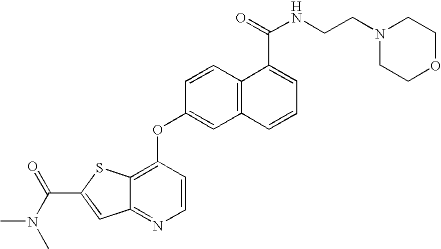

### A. Preparation of Intermediate 12a: 6-Hydroxy-N-(2-morpholin-4-ylethyl)-1-naphthamide

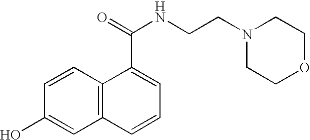

To a stirred solution of 6-hydroxy-1-naphthoic acid (5 g, 26.6 mmol) in DMF (30 mL) was added 2-morpholin-4-ylethanamine (3.5 mL, 26.6 mmol) followed by N-methyl morpholine (3.5 mL, 31.9 mmol), EDCI (5.6 g, 29.3 mmol) and HOBt (3.9 g, 29.3 mmol) sequentially, and the resulting slurry was stirred at ambient temperature for 18 hours. The resulting solution was concentrated in vacuo and pre-absorbed onto SiO₂, purified by flash chromatography (eluting with 95:5:0.5 DCM:MeOH:NH₃) to yield the crude product as a red foam. The crude was treated with decolorizing charcoal then the resulting pink solid was triturated with diethyl ether to yield the title compound, 6 g, 75%, as an off-white solid. ¹H NMR (400 MHz, DMSO-d₆) δ 9.87 (1H, s), 8.39 (1H, t, J=5.8 Hz), 8.18 (1H, d, J=9.1 Hz), 7.79 (1H, d, J=8.1 Hz), 7.44 (1H, t, J=8.0 Hz), 7.34 (1H, d, J=6.8 Hz), 7.19 (1H, d, J=2.5 Hz), 7.16 (1H, dd, J=2.3, 9.1 Hz), 3.64 (4H, t, 4.5 Hz), 3.46 (2H, q, J=6.3 Hz), 2.53-2.56 (6H, m). APCI m/z: 301.1 [MH+]. Anal. Calc'd. for C₁₇H₂₀N₂O₃: C, 67.98; H, 6.71; N, 9.33. Found: C, 67.56; H, 6.73; N, 9.28.

B. Preparation of Title Compound

The compound of Example 12 was prepared by the reaction of 7-chloro-N,N-dimethylthieno[3,2-b]pyridine-2-carboxamide (2a) (0.057 g, 0.24 mmol) with 6-hydroxy-N-(2-morpholin-4-ylethyl)-1-naphthamide (12a) (0.071 g, 0.24 mmol) and Cs₂CO₃ (0.117 g, 0.36 mmol) in a manner as described previously in Method C to give a white solid (0.066 g, 55%); ¹H NMR (300 MHz, CDCl₃) δ 8.49 (m, 2H), 7.85 (d, 1H, J=8.29 Hz), 7.72 (s, 1H), 7.62 (m, 2H), 7.51 (d, 1H, J=7.72 Hz), 7.36 (dd, 1H, J=9.23, 2.07 Hz), 6.79 (t, 1H, J=4.52 Hz), 6.63 (d, 1H, J=5.46 Hz), 3.69 (m, 6H), 3.27 (s, 3H), 3.15 (s, 3H), 2.64 (t, 2H, J=5.84 Hz), 2.52 (m, 4H). Anal. Calc'd. For C₂₇H₂₈N₄O₄S.0.22CHCl₃: C, 61.58; H, 5.36; N, 10.55; Found: C, 61.59; H, 5.74; N, 10.21.

### Example 13

### Preparation of 6-[(2{[(3R)-3-Hydroxypyrrolidin-1-yl]carbonyl}thieno[3,2-b]pyridin-7-yl)oxy]-N-(2-morpholin-4-ylethyl)-1-naphthamide

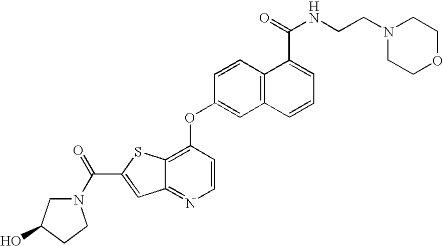

### A. Preparation of Intermediate 13a: (3R)-1-[(7-chlorothieno[3,2-b]pyridin-2-yl)carbonyl]pyrrolidin-3-ol

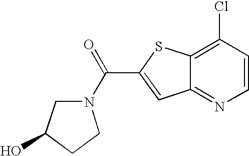

This material was prepared by the reaction of 7-chloro-thieno[3,2-b]pyridine-2-carboxylic acid lithium salt and 3R-hydroxypyrrolidine in a manner as described in Method A to give the desired amide. ¹H NMR (300 MHz, DMSO-d₆) δ 8.73 (1H, d, J=5.1 Hz), 8.12 (1H, d, J=14.3 Hz), 7.69 (1H, d, J=5.1 Hz), 5.10-5.06 (1H, m), 4,43-4.29 (1H, m), 4.05-3.89 (2H, m), 3.72-3.43 (2H, m), 2.08-1.79 (2H, m).

B. Preparation of Title Compound

The compound of Example 13 was prepared by the reaction of 6-hydroxy-N-(2-morpholin-4-ylethyl)-1-naphthamide (12a) (106 mg, 0.35 mmol) with (3R)-1-[(7-chlorothieno[3,2-b]pyridin-2-yl)carbonyl]pyrrolidin-3-ol (13a) (100 mg, 0.35 mmol) and Cs₂CO₃ (346 mg, 1.05 mmol), in a manner as described previously in Method D to yield the title compound, 174 mg, 90%, as a white foam. ¹H NMR (400 MHz, DMSO-d₆) δ 8.63 (1H, d, J=5.3 Hz), 8.56 (1H, t, J=5.9 Hz), 8.46 (1H, d, J=9.1 Hz), 8.12, 8.05 (1H, d), 8.05 (1H, m), 7.93 (1H, d, J=2.6 Hz), 7.63 (2H, d, J=4.5 Hz), 7.58 (1H, dd, J=2.5, 9.4 Hz), 6.87 (1H, d, J=5.3 Hz), 5.10 (1H, m), 4.42, 4.36 (1H, bd), 3.95-4.05 (2H, m), 3.60-3.72 (6H, m), 3.47-3.52 (2H, m), 2.45-2.60 (6H, m), 1.85-2.10 (2H, m). HRMS calc: 547.2010, found: 547.1996 [MH+]. Anal. Calc'd. for C₂₉H₃₀N₄O₅S.H₂O: C, 61.69; H, 5.71; N, 9.92. Found: C, 61.80; H, 5.63; N, 9.75.

### Example 14

### Preparation of 6-{[2-(1-Methyl-1H-imidazol-2-yl)thieno[3,2-b]pyridin-7-yl]oxy}-N-(2-morpholin-4-ylethyl)-1-naphthamide

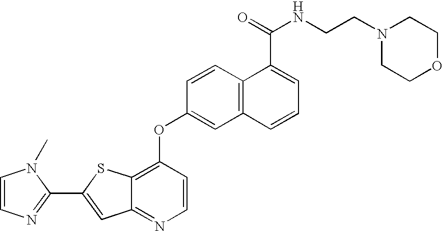

The compound of Example 14 was prepared by the reaction of 6-hydroxy-N-(2-morpholin-4-ylethyl)-1-naphthamide (12a) (106 mg, 0.35 mmol) with 7-chloro-2-(1-methyl-1H-imidazol-2-yl)thieno[3,2-b]pyridine (as prepared in PCT application WO 99/24440, Example 150) (88 mg, 0.35 mmol) and Cs₂CO₃ (346 mg, 1.05 mmol) in a manner as described previously in Method D followed by recrystalization from hot acetonitrile (6 mL) to yield the title compound, 78 mg, 43%, as tan crystals. ¹H NMR (400 MHz, CDCl₃) δ 8.51 (1H, s), 8.48 (1H, d, J=3.3 Hz), 7.87 (1H, d, J=8.3 Hz), 7.71 (1H, s), 7.63 (2H, d, J=2.6 Hz), 7.52 (1H, t, J=8.3 Hz), 7.41 (1H, dd, J=2,5, 9.3 Hz), 7.14 (1H, d, 1.1 Hz), 7.02 (1H, s), 6.64 (1H, d, J=5.5 Hz), 3.97 (3H, s), 3.68-3.75 (6H, m), 2.69 (2H, bs), 2.56 (4H, bs). HRMS calc: 514.1908, found: 514.1898 [MH+]. Anal. Calc'd. for C₂₈H₂₇N₅O₃S: C, 65.48; H, 5.30; N, 13.64. Found: C, 65.42; H, 5.26; N, 13.78.

### Example 15

### Preparation of N-(2-Morpholin-4-ylethyl)-6-(thieno[3,2-b]pyridin-7-yloxy)-1-naphthamide

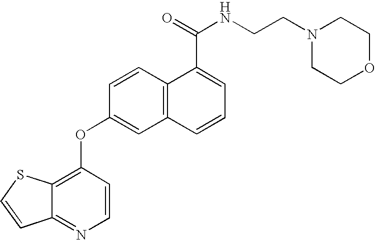

The compound of Example 15 was prepared from the reaction of 6-hydroxy-N-(2-morpholin-4-ylethyl)-1-naphthamide (12a) (100 mg, 0.33 mmol) with 7-chlorothieno[3,2-b]pyridine (as prepared in PCT application WO 99/24440) (57 mg, 0.33 mmol) and Cs₂CO₃ (346 mg, 1.05 mmol) in a manner as described previously in Method D to afford the title compound, 100 mg, 69%, as a cream solid. ¹H NMR (400 MHz, DMSO-d₆) δ 8.53 (1H, d, J=5.3 Hz), 8.52 (1H, q, J=5.9 Hz), 8.42 (1H, d, J=9.1 Hz), 8.16 (1H, d, J=5.3 Hz), 8.01 (2H, t, J=4.8 Hz), 7.88 (1H, d, J=2.5 Hz), 7.59-7.62 (3H, m), 7.53 (1H, dd, J=2.6, 9.3 Hz), 6.73 (1H, d, 5.5 Hz), 3.59 (4H, m), 3.45 (2H, m), 2.45-2.54 (6H, m). APCI m/z: 434.1 [MH+]. Anal. Calc'd. for C₂₄H₂₃N₃O₃S: C, 66.49; H, 5.35; N, 9.69. Found: C, 66.38; H, 5.37; N, 9.66.

### Example 16

### Preparation of 6-[(2-Chloropyrimidin-4-yl)oxy]-N-(2-morpholin-4-ylethyl)-1-naphthamide

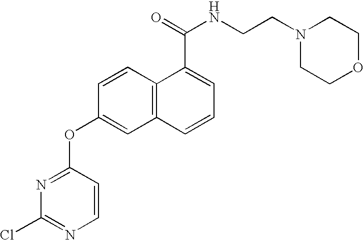

To a stirred suspension of 2,4-dichloro-pyrimidine (0.6 g, 4.03 mmol) and 6-hydroxy-N-(2-morpholin-4-ylethyl)-1-naphthamide (12a) (1 g, 3.36 mmol) in acetonitrile (10 mL) at ambient temperature was added DBU (0.6 ml, 4.04 mmol) dropwise. The resulting yellow solution was stirred at ambient temperature for 1 hour before concentrating in vacuo and the resulting residue was purified by flash chromatography (eluting with 97:3:0.3 DCM:MeOH:NH₃) to afford the title compound, 1.3 g, 95%, as a white foam. ¹H NMR (400 MHz, CDCl₃) δ 8.48 (2H, t, J=8.6 Hz), 7.91 (1H, d, J=8.4 Hz), 7.66 (2H, d, J=2.3 Hz), 7.54 (1H, t, J=7.0 Hz), 7.36 (1H, dd, J=2.6, 9.1 Hz), 6.87 (1H, d, J=5.8 Hz), 6.6 (1H, bs), 3.69-3.76 (6H, m), 2.69 (2H, bs), 2.57 (4H, bs). ESI m/z: 413.1 [MH+]. Anal. Calc'd. for C₂₁H₂₁N₄O₃Cl: C, 61.09; H, 5.13; N, 13.57. Found: C, 60.21; H, 5.15; N, 13.47.

### Example 17

### Preparation of N-(2-Morpholin-4-ylethyl)-6-(pyrimidin-4-yloxy)-1-naphthamide

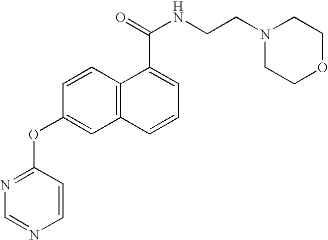

To a stirred solution of 6-[(2-chloropyrimidin-4-yl)oxy]-N-(2-morpholin-4-ylethyl)-1-naphthamide (Example 16) (89 mg, 0.22 mmol) in MeOH (5 mL) at ambient temperature under nitrogen, was added 10% Pd/C (20 mg) followed by ammonium formate (63 mg, 1.1 mmol). The resulting mixture was stirred for 16 hours at ambient temperature, then ammonium formate (13 mg, 0.22 mmol) was added, and the mix was stirred for another 1 hour. The catalyst was removed, and the solvents were evaporated in vacuo and the crude product was purified by flash chromatography (eluting with 95:5:0.5 DCM:MeOH:NH₃) to afford the title compound, 53 mg, 65%, as a white solid. ¹H NMR (400 MHz, CDCl₃) δ 8.78 (1H, s), 8.60 (1H, d, J=5.8 Hz), 8.48 (1H, d, 9.1 Hz), 7.89 (1H, d, 8.3 Hz), 7.63-7.65 (2 h, m), 7.51 (1H, dd, J=7.0, 7.1 Hz), 7.35 (1H, dd, J=2.5, 9.1 Hz), 6.97 (1H, dd, J=1.3, 5.8 Hz), 3.65-3.73 (6H, m), 2.67 (2H, bs), 2.55 (4H, bs). HRMS calc: 379.1765, found: 379.1756 [MH+]. Anal. Calcd. for C₂₁H₂₂N₄O₃.0.15H₂O: C, 66.18; H, 5.90; N, 14.70. Found: C, 66.21; H, 5.88; N, 14.59.

### Example 18

### Preparation of N-(2-Morpholin-4-ylethyl)-6-(pyridin-4-yloxy)-1-naphthamide

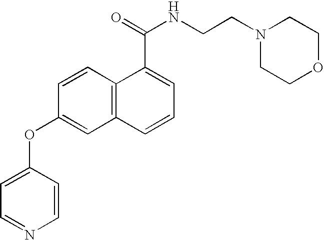

To a stirred suspension of 4-bromopyridine hydrochloride (100 mg, 0.51 mmol) and 6-hydroxy-N-(2-morpholin-4-ylethyl)-1-naphthamide (12a) (100 mg, 0.34 mmol) in DMA (10 mL) at ambient temperature was added Cs₂CO₃ (541 mg, 1.36 mmol). The resulting suspension was stirred at 110° C. for 18 hours before cooling to ambient temperature and filtering off inorganics. The residue was suspended in ethyl acetate (20 mL) and the precipitate filtered off, and the filtrate was concentrated in vacuo. The resulting residue was purified by flash chromatography (eluting with 95:5:0.5 DCM:MeOH:NH₃) to afford the title compound, 90 mg, 72%, as a white foam.

¹H NMR (400 MHz, CDCl₃) δ 8.46-8.49 (3H, m), 7.86 (1H, d, J=8.3 Hz), 7.62 (1H, d, J=7.1 Hz), 7.49-7.54 (2H, m), 7.32 (1H, dd, J=2.3, 9.1 Hz), 6.88 (1H, d, J=6.0 Hz), 6.85 (1H, bs), 3.66-3.73 (6H, m), 2.68 (2H, m), 2.56 (4H, bs). HRMS calc: 378.1812, found: 378.1803 [MH+]. Anal. Calcd. for C₂₂H₂₃N₃O₃.0.45H₂O: C, 68.54; H, 6.25; N, 10.90. Found: C, 68.55; H, 6.09; N, 10.90.

### Example 19

### Preparation of 6-[(6-Chloropyrimidin-4-yl)oxy]-N-(2-morpholin-4-ylethyl)-1-naphthamide

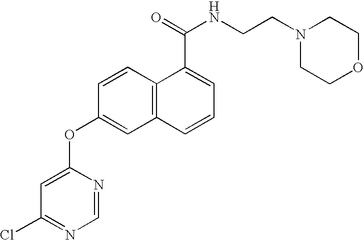

The compound of Example 19 was prepared by the reaction of 6-hydroxy-N-(2-morpholin-4-ylethyl)-1-naphthamide (12a) (1 g, 3.3 mmol) with 4,6-dichloro pyrimidine (0.65 g, 4.3 mmol) and DBU (0.8 mL, 5.3 mmol) in a manner as described previously for the preparation of the compound of Example 16 to afford the title compound, 1.22 g, 89%, as an off white solid. ¹H NMR (400 MHz, CDCl₃) δ 8.57 (1H, s), 8.49 (1H, d, J=9.1 Hz), 7.90 (1H, d, J=8.3 Hz), 7.64 (2H, m), 7.52 (1 h, t, J=7.0 Hz), 7.34 (1H, dd, J=2.6, 9.1 Hz), 6.98 (1H, s), 6.58 (1H, bs), 3.64-3.71 (6H, m), 2.65 (2H, bs), 2.53 (4H, bs). ESI m/z: 413.1 [MH+]. Anal. Calcd. for C₂₁H₂₁N₄O₃Cl: C, 61.09; H, 5.13; N, 13.57. Found: C, 60.96; H, 5.13; N, 13.60.

### Example 20

### Preparation of 6-{[6-(Methylamino)pyrimidin-4-yl]oxy}-N-(2-morpholin-4-ylethyl)-1-naphthamide

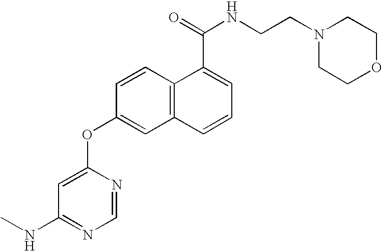

To a solution of 6-[(6-chloropyrimidin-4-yl)oxy]-N-(2-morpholin-4-ylethyl)-1-naphthamide (Example 19) (106 mg, 0.26 mmol) in NMP (0.1 mL) was added methylamine (2.0 M in THF, 256 uL, 0.52 mmol). The reaction was heated in a pre-heated oil bath at 80° C. for 30 minutes. The solvents were removed in vacuo and the residue was purified by flash chromatography (eluting with 95:5:0.5 DCM:MeOH:NH₃) to afford the title compound, 62 mg, 59%, as a white solid. ¹H NMR (400 MHz, DMSO-d₆) δ 8.46 (1H, bt), 8.32 (1H, d, J=9.3 Hz), 8.13 (1H, bs), 7.97 (1H, m), 7.71 (1H, d, J=2.5 Hz), 7.53-7.57 (2H, m), 7.35 (1H, dd, J=2.3, 9.3 Hz), 7.31 (1H, bm), 5.85 (1H, bs), 3.59 (4H, m), 3.45 (2H, m), 2.77 (3H, bs), 2.45-2.53 (6H, m). HRMS calc: 408.2030, found: 408.2015 [MH+]. Anal. Calcd. for C₂₂H₂₅N₅O₃: C, 64.85; H, 6.18; N, 17.19. Found: C, 64.60; H, 6.20; N, 17.01.

### Example 21

### Preparation of 6-[(6-{[2-(Methylamino)ethyl]amino}pyrimidin-4-yl)oxy]-N-(2-morpholin-4-ylethyl)-1-naphthamide

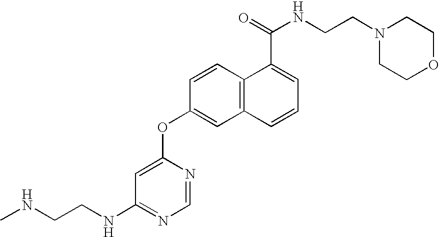

The compound of Example 21 was prepared by the reaction of 6-[(6-chloropyrimidin-4-yl)oxy]-N-(2-morpholin-4-ylethyl)-1-naphthamide (Example 19) (100 mg, 0.24 mmol) and N-methylethane-1,2-diamine (56 uL, 0.6 mmol) in a manner as described previously for the preparation of the compound of Example 20 to afford the title compound, 71 mg, 65%, as a white foam. ¹H NMR (400 MHz, CDCl₃) δ 8.40 (1H, d, J=9.1 Hz), 8.27 (1H, s), 7.86 (1H, d, J=8.3 Hz), 7.56-7.60 (2H, m), 7.47 (1H, t, J=8.1 Hz), 7.35 (1H, dd, J=2.3, 9.3 Hz), 6.51 (1H, bt), 5.92 (1H, s), 3.62-3.70 (9H, m), 3.04 (3H, s), 2.95 (2H, m), 2.62 (2H, t, J=6.0 Hz), 2,50 (4H, bs). HRMS calc: 451.2452, found: 451.2431 [MH+]. Anal. Calcd. for C₂₄H₃₀N₆O₃.0.75H₂O: C, 62.12; H, 6.84; N, 18.11. Found: C, 62.24; H, 6.81; N, 18.06.

### Example 22

### Preparation of N-(2-Morpholin-4-ylethyl)-6-[({6-[(2-pyrrolidin-1-ylethyl)amino]pyrimidin-4-yl}oxy)-1-naphthamide

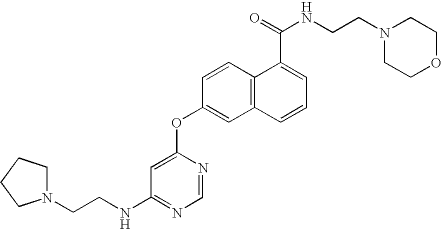

The compound of Example 22 was prepared by the reaction of 6-[(6-chloropyrimidin-4-yl)oxy]-N-(2-morpholin-4-ylethyl)-1-naphthamide (Example 19) (100 mg, 0.24 mmol) and 2-pyrrolidin-1-ylethanamine (77 ul, 0.6 mmol) in a manner as described previously for the preparation of the compound of Example 20 to afford the title compound, 43 mg, 36%, as a white foam. ¹H NMR (400 MHz, CDCl₃) δ 8.41 (1H, d, J=9.3 Hz), 8.25 (1H, s), 7.87 (1H, d, J=8.4 Hz), 7.57-7.60 (2H, m), 7.48 (1H, t, J=7.1 Hz), 7.33 (1H, dd, J=2.5, 9.1 Hz), 6.51 (1H, bt), 5.77 (1H, s), 5.73, (1H, bs), 3.63-3.70 (8H, m), 3.35 (2H, bs), 2.51-2.75 (14H, m). M/z: ESi 491.2 [MH+]. Anal. Calcd. for C₂₇H₃₄N₆O₃.0.5H₂O: C, 64.91; H, 7.06; N, 16.82. Found: C, 64.95; H, 7.04; N, 16.78.

### Example 23

### Preparation of N-(2-Morpholin-4-ylethyl)-6-({6-[(pyridin-3-ylmethyl)amino]pyrimidin-4-yl}oxy)-1-naphthamide

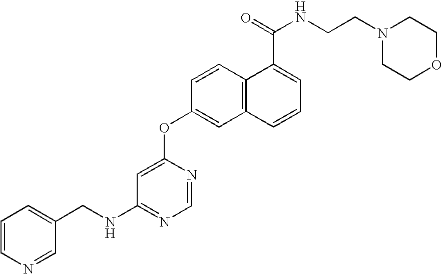

The compound of Example 23 was prepared by the reaction of 6-[(6-chloropyrimidin-4-yl)oxy]-N-(2-morpholin-4-ylethyl)-1-naphthamide (Example 19) (106 mg, 0.26 mmol) and 1-pyridin-4-ylmethanamine (66 uL, 0.64 mmol) in a manner as described previously for the preparation of the compound of Example 20 to afford the title compound, 71 mg, 57%, as a white foam. ¹H NMR (400 MHz, CDCl₃) δ 8.54 (1H, d, J=1.8 Hz), 8.51 (1H, dd, J=1.3, 4.8 Hz), 8.40 (1H, d, J=9.1 Hz), 8.27 (1H, s), 7.85 (1H, d, J=8.0 Hz), 7.56-7.63 (3H, m), 7.47 (1H, t, J=7.0 Hz), 7.30 (1H, dd, J=2.6, 9.4 Hz), 7.23-7.27 (1H, m), 6.64(1H, bs), 5.77 (1H, s), 5.49 (1H, bs), 4.53 (2H, d, J=5.8 Hz), 3.70 (4H, m), 3.64 (2H, m), 2.64 (2H, m), 2.53 (4H, bs). HRMS calc: 485.2296, found: 485.2288 [MH+]. Anal. Calcd. for C₂₇H₂₈N₆O₃.0.44H₂O: C, 65.85; H, 5.91; N, 17.06. Found: C, 65.80; H, 5.84; N, 17.08.

### Example 24

### Preparation of N-(2-Morpholin-4-ylethyl)-6-({6-[(2-pyridin-4-ylethyl)amino]pyrimidin-4-yl}oxy)-1-naphthamide

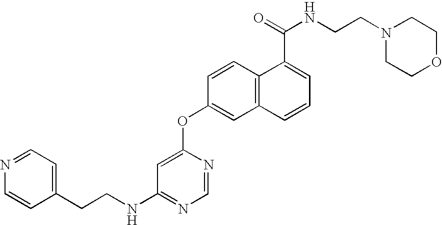

The compound of Example 25 was prepared by the reaction of 6-[(6-chloropyrimidin-4-yl)oxy]-N-(2-morpholin-4-ylethyl)-1-naphthamide (Example 19) (106 mg, 0.26 mmol) and 2-pyridin-4-ylethanamine(125 mg, 1.04 mmol) in a manner as described previously for the preparation of the compound of Example 20 to afford the title compound, 66 mg, 52%, as a yellow foam. ¹H NMR (400 MHz, CDCl₃) δ 8.51 (2H, d, J=6.1 Hz), 8.42 (1H, d, J=9.1 Hz), 8.27 (1H, s), 7.85 (1H, d, J=8.3 Hz), 7.59-7.63 (2H, m), 7.49 (1H, t, J=8.3 Hz), 7.32 (1H, dd, J=2.5, 9.1 Hz), 7.12 (1H, d, J=6.0 Hz), 6.67(1H, bs), 5.75 (1H, s), 4.99 (1H, bs), 3.72 (4H, m), 3.67 (2H, m), 3.58 (2H, bm), 2.90 (2H, t, J=7.0 Hz), 2.67 (2H, bm), 2.56 (4H, bm). M/z: APCI 499.2 [MH+]. Anal. Calcd. For C₂₈H₃₀N₆O₃.0.14H₂O: C, 67.11; H, 6.09; N, 16.77. Found: C, 67.15; H, 6.15; N, 16.44.

### Example 25

### Preparation of 6-{[2-(Methylamino)pyrimidin-4-yl]oxy}-N-(2-morpholin-4-ylethyl)-1-naphthamide

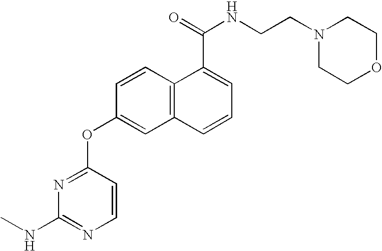

The compound of Example 25 was prepared by the reaction of 6-[(2-chloropyrimidin-4-yl)oxy]-N-(2-morpholin-4-ylethyl)-1-naphthamide (Example 16) (106 mg, 0.26 mmol) and methylamine (2.0 M in THF, 256 uL, 0.52 mmol) in a manner as described previously for the preparation of the compound of Example 20 to afford the title compound, 56 mg, 53%, as an off white solid. ¹H NMR (400 MHz, DMSO-d₆) δ 8.33 (1H, t), 8.17 (1H, d, J=9.1 Hz), 8.06 (1H, bs), 7.85 (1H, m), 7.64 (1H, bs), 7.40-7.44 (2H, m), 7.28 (1H, m), 6.86 (1H, bs), 6.08 (1H, bs), 3.46 (4H, t, J=4.3 Hz), 3.31 (2H, m), 3.17 (3H, s), 2.32-2.41 (6H, m). HRMS calc: 408.2030, found: 408.2010 [MH+]. Anal. Calcd. for C₂₂H₂₅N₅O₃: C, 64.85; H, 6.18; N, 17.19. Found: C, 64.71; H, 6.29; N, 17.02.

### Example 26

### Preparation of 6-[(2-{(2-(Methylamino)ethyl]amino}pyrimidin-4-yl)oxy]-N-(2-morpholin-4-ylethyl)-1-naphthamide

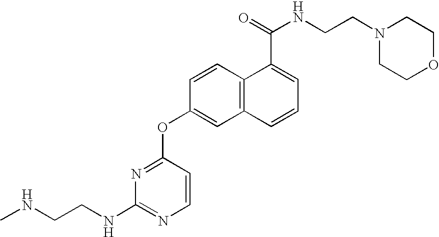

The compound of Example 26 was prepared by the reaction of 6-[(2-chloropyrimidin-4-yl)oxy]-N-(2-morpholin-4-ylethyl)-1-naphthamide (Example 16) (100 mg, 0.24 mmol) and N-methylethane-1,2-diamine (54 uL, 0.61 mmol) in a manner as described previously for the preparation of the compound of Example 20 to afford the title compound, 41 mg, 38%, as a hygroscopic white foam. ¹H NMR (400 MHz, CDCl₃) δ 8.41 (1H, d, J=9.0 Hz), 8.18 (1H, d, J=5.5 Hz), 7.87 (1H, d, J=8.1 Hz), 7.58-7.62 (2H, m), 7.49 (1H, t, J=7.3 Hz), 7.37 (1H, dd, J=2.3, 9.1 Hz), 6.67 (1H, bs), 6.09 (1H, d, 5.3 Hz), 3.63-3.71 (7H, m), 3.46 (2H, bm), 3.04 (3H, bs), 2.78 (2H, bs), 2.64 (2H, d, J=6.1 Hz), 2.52 (4H, bs). HRMS calc: 451.2452, found: 451.2441 [MH+]. Anal. Calcd. for C₂₄H₃₀N₆O₃.0.6H₂O: C, 62.48; H, 6.82; N, 18.22. Found: C, 62.49; H, 6.73; N, 18.20.

### Example 27

### Preparation of N-(2-Morpholin-4-ylethyl)-6-({2-[(2-pyrrolidin-1-ylethyl)amino]pyrimidin-4-yl}oxy)-1-naphthamide

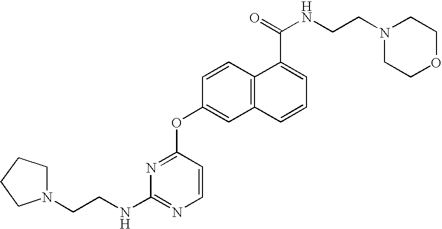

The compound of Example 27 was prepared by the reaction of 6-[(2-chloropyrimidin-4-yl)oxy]-N-(2-morpholin-4-ylethyl)-1-naphthamide (Example 16) (110 mg, 0.27 mmol) and 2-pyrrolidin-1-ylethanamine (85 uL, 0.68 mmol) in a manner as described previously for the preparation of the compound of Example 20 to afford the title compound, 67 mg, 51%, as an of white solid. ¹H NMR (400 MHz, CDCl₃) δ8.41 (1H, d, J=9.1 Hz), 8.14 (1H, d, J=5.3 Hz), 7.88 (1H, d, J=8.1 Hz), 7.58-7.62 (2H, m), 7.49 (1H, t, J=8.1 Hz), 7.36 (1H, dd, J=2.5, 9.3 Hz), 6.56 (1H, bs), 6.10 (1H, d, 5.5 Hz), 5.56 (1H, bs), 3.63-3.71 (8H, m), 3.38 (2H, bm), 2.44-2.65 (14H, m). HRMS calc: 491.2765, found: 491.2732 [MH+]. Anal. Calcd. for C₂₇H₃₄N₆O₃.0.2H₂O: C, 65.62; H, 7.02; N, 17.00. Found: C, 65.63; H, 7.08; N, 17.14.

### Example 28

### Preparation of N-(2-Morpholin-4-ylethyl)-6-({2-[(pyridin-3-ylmethyl)amino]pyrimidin-4-yl}oxy)-1-naphthamide

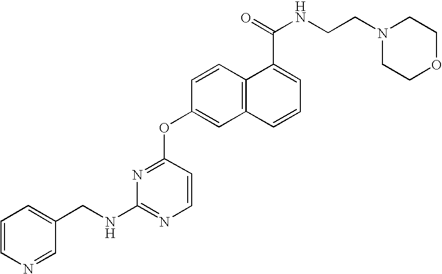

The compound of Example 28 was prepared by the reaction of 6-[(2-chloropyrimidin-4-yl)oxy]-N-(2-morpholin-4-ylethyl)-1-naphthamide (Example 16) (100 mg, 0.24 mmol) and 1-pyridin-4-ylmethanamine (200 uL, 1.94 mmol) in a manner as described previously for the preparation of the compound of Example 20 to afford the title compound, 90 mg, 75%, as an off white foam. ¹H NMR (400 MHz, CDCl₃) δ8.40 (1H, d, J=9.4 Hz), 8.36 (1H, bs), 8.15 (1H, d, J=5.5 Hz), 7.85 (1H, d, J=8.1 Hz), 7.64 (1H, d, J=6.8 Hz), 7.55 (1H, d, J=2.3 Hz), 7.50 (1H, t, J=7.1 Hz), 7.43 (1H, bs), 7.10 (1H, bs), 6.23(1H, d, J=5.6 Hz), 5.54 (1H, bs), 4.45 (1H, bs), 3.70-3.73 (6H, bs), 2.70 (2H, bm), 2.87 (4H, bm). M/z API-ES (pos): 485.1 [MH+]. Anal. Calcd. for C₂₇H₂₈N₆O₃.0.45H₂O: C, 65.83; H, 5.91; N, 17.06. Found: C, 65.80; H, 5.80; N, 17.17.

### Example 29

### Preparation of 6-[2-(1-Methyl-1H-imidazol-2-yl)-thieno[3,2-b]pyridin-7-yloxy]-naphthalene-1-carboxylic acid (2-pyrrolidin-1-yl-ethyl)-amide

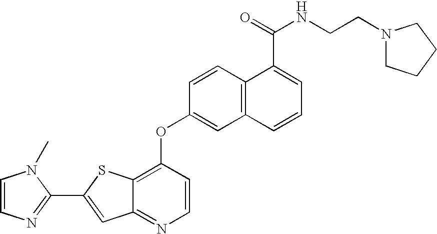

### A. Preparation of Intermediate 29a: 6-[2-(1-Methyl-1H-imidazol-2-yl)-thieno[3,2-b]pyridin-7-yloxy]-naphthalene-1-carboxylic acid

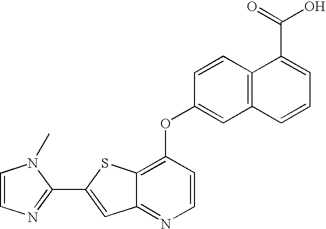

This material was prepared by the reaction of 7-chloro-2-(1-methyl-1H-imidazol-2-yl)thieno[3,2-b]pyridine (200 mg, 0.803 mmol) with 6-hydroxy-1-naphthoic acid (151 mg, 0.803 mmol) and Cs₂CO₃ (658 mg, 2.01 mmol) in a manner as described previously in Method C to give the title compound (150 mg) as a brown solid. ¹H NMR (300 MHz, DMSO-d₆) δ 9.09 (bs, 1H), 8.54 (bs, 1H), 8.14 (d, 2H, J=8.66 Hz), 7.99-7.88 (m, 2H), 7.67-7.55 (m, 2H), 7.40 (s, 1H), 7.02 (s, 1H), 6.80 (d, 1H, J=5.46 Hz), 3.98 (s, 3H). LCMS (ESI+) [M+H]/z Calc'd 402, found 402.

B. Preparation of Title Compound

The compound of Example 29 was prepared by the reaction of 6-[2-(1-methyl-1H-imidazol-2-yl)-thieno[3,2-b]pyridin-7-yloxy]-naphthalene-1-carboxylic acid (29a) (50 mg, 0.124 mmol) and 1-(2-aminoethyl)-pyrrolidine (42 mg, 0.372 mmol) in a manner as described previously in Method A to give 22 mg of the title compound as a white solid. ¹H NMR (300 MHz, CD₃OD) δ 8.42 (d, 1H, J=5.65 Hz), 8.35 (d, 1H, J=9.23 Hz), 7.92 (d, 1H, J=8.28 Hz), 7.74-7.70 (m, 2H), 7.62 (d, 1H, J=6.97 Hz), 7.52 (d, 1H, J=7.54 Hz), 7.44-7.36 (m, 1H), 7.23 (s, 1H), 7.00 (s, 1H), 6.69 (d, 1H, J=5.65 Hz), 3.94 (s, 3H), 3.58 (t, 2H, J=6.78 Hz), 2.81 (t, 2H, J=6.78 Hz), 2.74-2.65 (m, 4H), 1.84-1.76 (m, 4H). LCMS (ESI+) [M+H]/z Calc'd 498, found 498. Anal. (C₂₈H₂₇N₅O₂S.0.8CH₃COOH)C, H, N.

### Example 30

### Preparation of 6-[2-(1-Methyl-1H-imidazol-2-yl)-thieno[3,2-b]pyridin-7-yloxy]-naphthalene-1-carboxylic acid (3-morpholin-4-yl-propyl)-amide

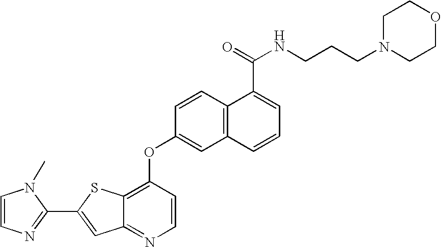

The compound of Example 30 was prepared by the reaction of 6-[2-(1-methyl-1H-imidazol-2-yl)-thieno[3,2-b]pyridin-7-yloxy]-naphthalene-1-carboxylic acid (29a) and 4-(3-aminopropyl)-morpholine in a manner as described previously in Method A to give the title compound. ¹H NMR (300 MHz, CD₃OD) δ 8.40 (d, 1H, J=5.47 Hz), 8.28 (d, 1H, J=9.23 Hz), 7.90 (d, 1H, J=7.91 Hz), 7.74-7.69 (m, 2H), 7.59-7.43 (m, 2H), 7.42-7.34 (m, 1H), 7.21 (s, 1H), 6.99 (s, 1H), 6.67 (d, 1H, J=5.46 Hz), 3.92 (s, 3H), 3.59-3.50 (m, 4H), 3.47-3.36 (m, 2H), 2.49-2.35 (m, 6H), 1.87-1.75 (m, 2H). LCMS (ESI+) [M+H]/z Calc'd 528, found 528. Anal. (C₂₉H₂₉N₅O₃S.0.9CH₃COOH)C, H, N.

### Example 31

### Preparation of 6-[2-(Azetidine-1-carbonyl)-thieno[3,2-b]pyridin-7-yloxy]-naphthalene-1-carboxylic acid (2-morpholin-4-yl-ethyl)-amide

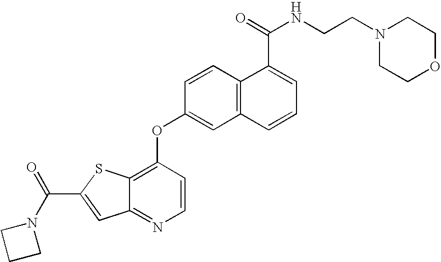

### A. Preparation of Intermediate 31a: Azetidin-1-yl-(7-chloro-thieno[3,2-b]pyridin-2-yl)-methanone

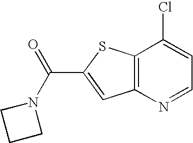

Intermediate 31a was prepared from 7-chloro-thieno[3,2-b]pyridine-2-carboxylic acid and azetidine hydrochloride following Method A as described previously. ¹H NMR (300 MHz, DMSO-d₆) δ 8.72 (1H, d, J=5.1 Hz), 7.96 (1H, s), 7.70 (1H, d, J=5.1 Hz), 4.62 (2H, t, J=7.4 Hz), 4.12 (2H, t, J=7.7 Hz), 2.34 (2H, tt, J=7.4, 7.7 Hz).

### B. Preparation of Intermediate 31b: 6-[2-(Azetidine-1-carbonyl)-thieno[3,2-b]pyridin-7-yloxy]-naphthalene-1-carboxylic acid

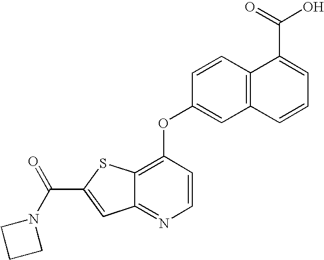

This material was prepared by the reaction of azetidin-1-yl-(7-chloro-thieno[3,2-b]pyridin-2-yl)-methanone (31a) with 6-hydroxy-1-naphthoic acid and Cs₂CO₃ in a manner as described previously in Method C to give the title compound. ¹H NMR (300 MHz, DMSO-d₆) δ 9.03 (d, 1H, J=9.42 Hz), 8.61 (d, 1H, J=5.27 Hz), 8.17 (d, 2H, J=7.53 Hz), 7.95 (d, 1H, J=2.82 Hz), 7.92 (s, 1H), 7.70 (d, 1H, J=2.26 Hz), 7.68-7.59 (m, 1H), 6.87 (d, 1H, J=5.47 Hz), 4.69-4.58 (m, 2H), 4.16-4.07 (m, 2H), 2.41-2.28 (m, 2H). LCMS (ESI+) [M+H]/z Calc'd 405, found 405.

C. Preparation of Title Compound

The compound of Example 31 was prepared by the coupling of 31b and 2-morpholin-4-ylethanamine in a manner as described previously in Method A to give the title compound. ¹H NMR (300 MHz, CD₃OD) δ 8.44 (d, 1H, J=5.65 Hz), 8.35 (d, 1H, J=9.23 Hz), 7.89 (d, 1H, J=7.92 Hz), 7.73 (s, 1H), 7.69 (d, 1H, J=2.26 Hz), 7.60-7.54 (m, 1H), 7.50-7.47 (m, 1H), 7.38-7.33 (m, 1H), 6.70 (d, 1H, J=5.47 Hz), 4.63-4.52 (m, 2H), 4.20-4.09(m, 2H), 3.66-3.58 (m, 4H), 3.57-3.49 (m, 2H), 2.63-2.55 (m, 2H), 2.54-2.43 (m, 4H), 2.43-2.33 (m, 2H). LCMS (ESI+) [M+H]/z Calc'd 517, found 517. Anal. (C₂₈H₂₈N₄O₄S.0.6H₂O.0.9CH₃COOH)C, H, N.

### Example 32

### Preparation of 6-[2-(Azetidine-1-carbonyl)-thieno[3,2-b]pyridin-7-yloxy]-naphthalene-1-carboxylic acid (3-morpholin-4-yl-propyl)-amide

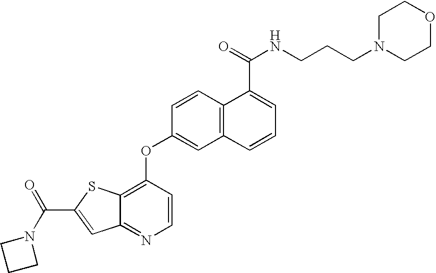

The compound of Example 32 was prepared by the coupling of intermediate 31b and 4-(3-aminopropyl)-morpholine in a manner as described previously in Method A to give the title compound. ¹H NMR (300 MHz, CD₃OD) δ 8.45 (d, 1H, J=5.46 Hz), 8.29 (d, 1H, J=9.23 Hz), 7.90 (d, 1H, J=8.10 Hz), 7.73 (s, 1H), 7.70 (d, 1H, J=2.26 Hz), 7.60-7.54 (m, 1H), 7.53-7.45 (m, 1H), 7.41-7.33 (m, 1H), 6.70 (d, 1H, J=5.46 Hz), 4.65-4.52 (m, 2H), 4.22-4.10 (m, 2H), 3.61-3.52 (m, 4H), 3.48-3.39 (m, 2H), 2.50-2.32 (m, 8H), 1.87-1.74 (m, 2H). LCMS (ESI+) [M+H]/z Calc'd 531, found 531. Anal. (C₂₉H₃₀N₄O₄S.0.5CH₃COOH)C, H, N.

### Example 33

### Preparation of 6-[2-((R)-3-Methoxy-pyrrolidine-1-carbonyl)-thieno[3,2-b]pyridin-7-yloxy]-naphthalene-2-carboxylic acid methylamide

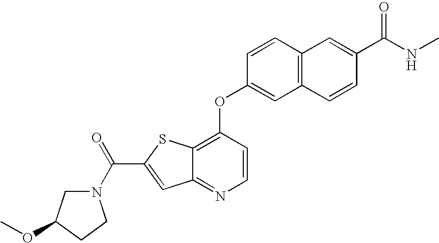

### A. Preparation of Intermediate 33a: 7-Chloro-2-{[(3R)-3-methoxypyrrolidin-1-yl]carbonyl}thieno[3,2-b]pyridine

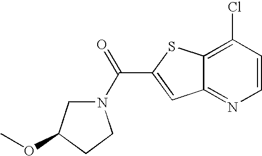

This material was prepared by the reaction of 7-chloro-thieno[3,2-b]pyridine-2-carboxylic acid lithium salt and 3R-methoxy-pyrrolidine in a manner as described previously in Method A to give the title compound. ¹H NMR (300 MHz, CDCl₃) δ 8.68 (d, 1H, J=5.5 Hz), 7.85 (d, 1H, J=14.3 Hz), 7.40 (d, 1H, J=5.5 Hz), 4.18-4.07 (m, 1H), 4.03-3.73 (m, 4H), 3.20 (d, 3H, J=14.5 Hz), 2.36-2.03 (m, 2H). LCMS ESI (M+H⁺): 297.05.

### B. Preparation of Intermediate 33b: 6-[2-((R)-3-Methoxy-pyrrolidine-1-carbonyl)-thieno[3,2-b]pyridin-7-yloxy]-naphthalene-2-carboxylic Acid

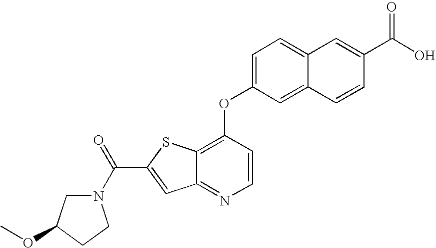

Intermediate 33b was prepared by the coupling of intermediate 33a and 6-hydroxy-2-naphthoic acid in a manner as described previously in Method C. ¹H NMR (300 MHz, DMSO-d₆) δ 13.12 (s, 1H), 8.71 (s, 1H), 8.63 (d, 1H, J=5.27 Hz), 8.30 (d, 1H, J=9.04 Hz), 8.10 (s, 1H), 8.03 (s, 2H), 7.92 (d, 1H, J=2.07 Hz), 7.63-7.57 (m, 1H), 6.91 (d, 1H, J=5.47 Hz), 4.12-4.00 (m, 3H), 4.00-3.83 (m, 2H), 3.63 (s, 3H), 2.21-1.94 (m, 2H). LCMS (ESI+) [M+H]/z Calc'd 449, found 449.

C. Preparation of Title Compound

The compound of Example 33 was prepared from the coupling of intermediate 33b and methylamine in a manner as described previously in Method A to give the title compound. ¹H NMR (300 MHz, CD₃OD) δ 8.46 (d, 1H, J=5.65 Hz), 8.35 (s, 1H), 8.04 (d, 1H, J=8.86 Hz), 7.89-7.79 (m, 3H), 7.70 (d, 1H, J=2.07 Hz), 7.42-7.34 (m, 1H), 6.73 (d, 1H, J=5.47 Hz), 4.06-3.82 (m, 3H), 3.74-3.56 (m, 2H), 3.28 (d, 3H, J=13.38 Hz), 2.90 (s, 3H), 2.21-1.94 (m, 2H). LCMS (ESI+) [M+H]/z Calc'd 462, found 462. Anal. (C₂₅H₂₃N₃O₄S.1.0CH₃COOH)C, H, N.

### Example 34

### Preparation of 6-(Thieno[3,2-b]pyridin-7-yloxy)-naphthalene-1-carboxylic acid (4-hydroxy-3,4,5,6-tetrahydro-2H-[1,2′]bipyridinyl-5′-yl)-amide

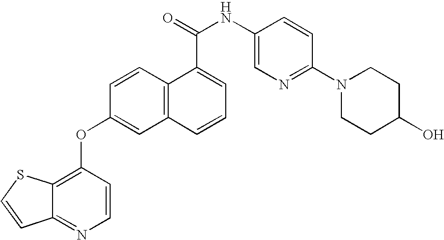

### A. Preparation of Intermediate 34a: 6-(Thieno[3,2-b]pyridin-7-yloxy)-naphthalene-1-carboxylic acid

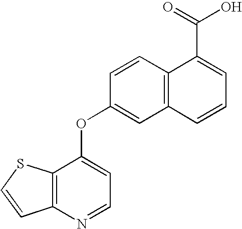

Intermediate 34a was prepared from 7-chlorothieno[3,2-b]pyridine and 6-hydroxy-1-naphthoic acid in a manner as described previously in Method C. ¹H NMR (300 MHz, CD₃OD) δ 8.67 (d, 1H, J=9.99 Hz), 8.50 (d, 1H, J=5.09), 8.02 (d, 1H, J=5.46 Hz), 7.90-7.84 (m, 1H), 7.79-7.67 (m, 1H), 7.51-7.45 (m, 1H), 7.40 (d, 1H, J=5.06 Hz), 7.36-7.28 (m, 1H), 7.10-7.04 (m, 1H), 6.67 (d, 1H, J=5.46 Hz). LCMS (ESI+) [M+H]/z Calc'd 322, found 322. Anal. (C₁₈H₁₁NO₃S.0.5H₂O)C, H, N.

B. Preparation of Title Compound

The compound of Example 34 was prepared from the reaction of 6-(thieno[3,2-b]pyridin-7-yloxy)-naphthalene-1-carboxylic acid (34a) (50 mg, 0.156 mmol) with 5′-amino-3,4,5,6-tetrahydro-2H-[1,2′]bipyridinyl-4-ol (36 mg, 0.187 mmol) and Et₃N (0.04 ml, 0.936 mmol) in a manner as described previously in Method A to provide 15.5 mg of the title compound as an yellow solid. ¹H NMR (300 MHz, CD₃OD) δ8.38 (d, 1H, J=5.56 Hz), 8.35 (s, 1H), 8.34 (d, 1H, J=7.07 Hz), 7.96-7.91 (m, 2H), 7.90-7.86 (m, 1H), 7.73-7.69 (m, 2H), 7.57-7.51 (m, 1H), 7.45 (d, 1H. J=5.55 Hz), 7.42-7.37 (m, 1H), 6.81 (d, 1H, J=9.35 Hz), 6.85 (d, 1H, J=5.56 Hz), 5.39 (s, 1H), 4.03-3.93 (m, 2H), 3.78-3.70 (m, 1H), 3.07-2.99 (m, 2H), 1.86-1.81 (m, 2H), 1.53-1.39 (m, 2H). LCMS (ESI+) [M+H]/z Calc'd 497, found 497. Anal. (C₂₈H₂₄N₄O₃S.0.4CH₂Cl₂) C, H, N.

### Example 35

### Preparation of 6-(Thieno[3,2-b]pyridin-7-yloxy)-naphthalene-1-carboxylic acid (6-morpholin-4-yl-pyridin-3-yl)-amide

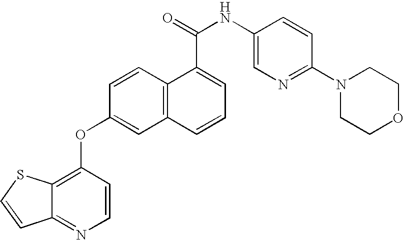

The compound of Example 35 was prepared by the reaction of 6-(thieno[3,2-b]pyridin-7-yloxy)-naphthalene-1-carboxylic acid (34a) with 6-morpholin-4-yl-pyridin-3-ylamine in a manner as described previously in Method A to give the title compound. ¹H NMR (300 MHz, CD₃OD) δ 8.38 (d, 1H, J=5.56 Hz), 8.35 (s, 1H), 8.34 (d, 1H, J=7.07 Hz), 7.96-7.91 (m, 2H), 7.90-7.86 (m, 1H), 7.73-7.69 (m, 2H), 7.57-7.51 (m, 1H), 7.45 (d, 1H, J=5.55 Hz), 7.42-7.37 (m, 1H), 6.81 (d, 1H, J=9.35 Hz), 6.85 (d, 1H, J=5.56 Hz), 3.80-3.64 (m, 4H), 3.47-3.34 (m, 4H). LCMS (ESI+) [M+H]/z Calc'd 483, found 483. Anal. (C₂₇H₂₂N₄O₃S.0.8H₂O)C, H, N.

### Example 36

### Preparation of 6-(Thieno[3,2-b]pyridin-7-yloxy)-naphthalene-1-carboxylic acid (3-piperidin-1-yl-propyl)-amide

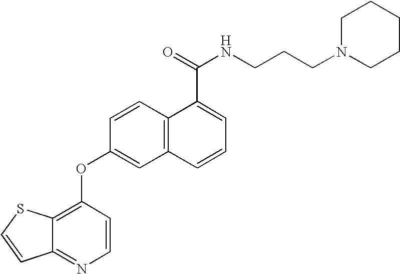

The compound of Example 36 was prepared by the reaction of 6-(thieno[3,2-b]pyridin-7-yloxy)-naphthalene-1-carboxylic acid (34a) with 3-piperidin-1-yl-propylamine in a manner as described previously in Method A to give the title compound. ¹H NMR (300 MHz, CD₃OD) δ 8.36 (d, 1H, J=5.57 Hz), 8.29 (d, 1H, J=9.23 Hz), 7.94-7.86 (m, 2H), 7.68 (d, 1H, J=2.26 Hz), 7.60-7.56 (m, 1H), 7.50 (d, 1H, J=8.10 Hz), 7.44 (d, 1H, J=5.46 Hz), 7.39-7.34 (m, 1H), 6.62 (d, 1H, J=5.46 Hz), 3.48-3.39 (m, 2H), 2.80-2.75 (m, 6H), 1.97-1.87 (m, 2H), 1.69-1.59 (m, 4H), 1.50-1.41 (m, 2H). LCMS (ESI+) [M+H]/z Calc'd 446, found 446. Anal. (C₂₆H₂₇N₃O₂S.0.35CH₂Cl₂) C, H, N.

### Example 37

### Preparation of 6-(Thieno[3,2-b]pyridin-7-yloxy)-naphthalene-1-carboxylic acid (3-pyrrolidin-1-yl-propyl)-amide

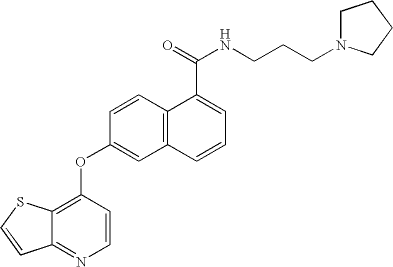

The compound of Example 37 was prepared by the reaction of 6-(thieno[3,2-b]pyridin-7-yloxy)-naphthalene-1-carboxylic acid (34a) with 3-pyrrolidin-1-yl-propylamine in a manner as described previously in Method A to give the title compound. ¹H NMR (300 MHz, CD₃OD) δ 8.35 (d, 1H, J=5.56 Hz), 8.28 (d, 1H, J=9.10 Hz), 7.92-7.86 (m, 2H), 7.67 (d, 1H, J=2.26 Hz), 7.59 (d, 1H, J=7.08 Hz), 7.51-7.45 (m, 1H), 7.43 (d, 1H, J=5.56 Hz), 7.38-7.33 (m, 1H), 6.61 (d, 1H, J=5.56 Hz), 3.48-3.43 (m, 2H), 3.15-3.01 (m, 6H), 2.00-1.89 (m, 6H). LCMS (ESI+) [M+H]/z Calc'd 432, found 432. Anal. (C₂₅H₂₅N₃O₂S.0.65CH₂Cl₂) C, H, N.

### Example 38

### Preparation of 6-(Thieno[3,2-b]pyridin-7-yloxy)-naphthalene-1-carboxylic acid [3-(4-methyl-piperazin-1-yl)-propyl]-amide

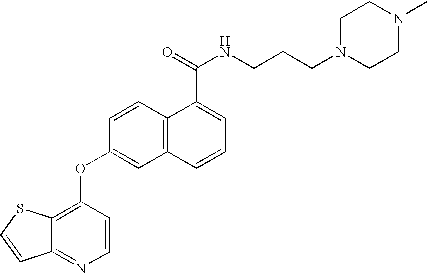

The compound of Example 38 was prepared by the reaction of 6-(thieno[3,2-b]pyridin-7-yloxy)-naphthalene-1-carboxylic acid (34a) with 3-(4-methyl-piperazin-1-yl)-propylamine in a manner as described previously in Method A to give the title compound. ¹H NMR (300 MHz, CD₃OD) δ 8.33 (d, 1H, J=5.56 Hz), 8.25 (d, 1H, J=9.09 Hz), 7.88 (d, 1H, J=5.31 Hz), 7.84 (d, 1H, J=8.08 Hz), 7.63 (d, 1H, J=2.53 Hz), 7.55-7.51 (m, 1H), 7.47-7.40 (m, 2H), 7.35-7.30 (m, 1H), 6.58 (d, 1H, J=5.56 Hz), 3.43-3.38 (m, 2H), 2.56-2.43 (m, 10H), 2.28 (s, 3H), 1.80-1.74 (m, 2H). LCMS (ESI+) [M+H]/z Calc'd 461, found 461. Anal. (C₂₆H₂₈N₄O₂S.0.3CH₂Cl₂) C, H, N.

### Example 39

### Preparation of 6-(Thieno[3,2-b]pyridin-7-yloxy)-naphthalene-1-carboxylic acid (3-morpholin-4-yl-propyl)-amide

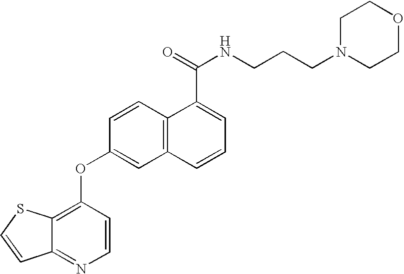

The compound of Example 39 was prepared by the reaction of 6-(thieno[3,2-b]pyridin-7-yloxy)-naphthalene-1-carboxylic acid (34a) with 3-morpholin-4-yl-propylamine in a manner as described previously in Method A to give the title compound. ¹H NMR (300 MHz, CD₃OD) δ 8.37 (d, 1H, J=5.56 Hz), 8.28 (d, 1H, J=9.35 Hz), 7.92 (d, 1H, J=5.56 Hz), 7.89 (d, 1H, J=8.34 Hz), 7.69 (d, 1H, J=2.53 Hz), 7.56-7.54 (m, 1H), 7.50-7.46 (m, 1H), 7.44 (d, 1H, J=5.56 Hz), 7.40-7.35 (m, 1H), 6.63 (d, 1H, J=5.55 Hz), 3.61-3.55 (m, 4H), 3.46-3.41 (m, 2H), 2.51-2.45 (m, 6H), 1.86-1.78 (m, 2H). LCMS (ESI+) [M+H]/z Calc'd 448, found 448. Anal. (C₂₅H₂₅N₃O₃S.0.2CH₂Cl₂) C, H, N.

### Example 40

### Preparation of 6-(Thieno[3,2-b]pyridin-7-yloxy)-naphthalene-1-carboxylic acid (3-dimethylamino-propyl)-amide

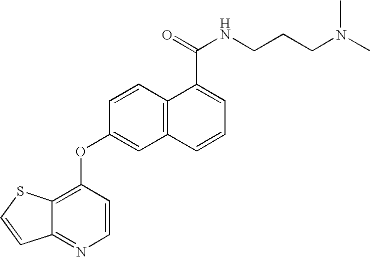

The compound of Example 40 was prepared by the reaction of 6-(thieno[3,2-b]pyridin-7-yloxy)-naphthalene-1-carboxylic acid (34a) with N,N′-dimethyl-propane-1,3-diamine in a manner as described previously in Method A to give the title compound. ¹H NMR (300 MHz, CD₃OD) δ 8.39 (d, 1H, J=5.65 Hz), 8.30 (d, 1H, J=9.23 Hz), 7.94-7.86 (m, 2H), 7.70 (d, 1H, J=2.45 Hz), 7.60-7.57 (m, 1H), 7.54-7.44 (m, 2H), 7.42-7.36 (m, 1H), 6.64 (d, 1H, J=5.65 Hz), 3.49-3.41 (m, 2H), 2.79-2.72 (m, 2H), 2.50 (s, 6H), 1.95-1.86 (m, 2H). LCMS (ESI+) [M+H]/z Calc'd 406, found 406. Anal. (C₂₃H₂₃N₃O₂S.0.65CH₂Cl₂) C, H. N.

### Example 41

### Preparation of 6-(Thieno[3,2-b]pyridin-7-yloxy)-naphthalene-1-carboxylic acid (2-dimethylamino-ethyl)-amide

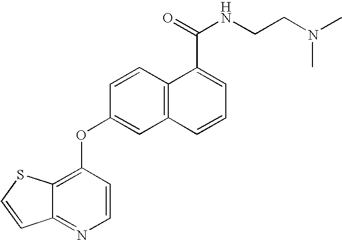

The compound of Example 41 was prepared by the reaction of 6-(thieno[3,2-b]pyridin-7-yloxy)-naphthalene-1-carboxylic acid (34a) with N′,N′-dimethyl-ethane-1,2-diamine in a manner as described previously in Method A to give the title compound. ¹H NMR (300 MHz, CD₃OD) δ 8.35 (d, 1H, J=5.56 Hz), 8.32 (d, 1H, J=9.35 Hz), 7.91-7.86 (m, 2H), 7.65 (d, 1H, J=2.52 Hz), 7.61 (d, 1H, J=6.32 Hz), 7.49-7.41 (m, 2H), 7.37-7.29 (m, 1H), 6.60 (d, 1H, J=5.56 Hz), 3.65-3.59 (m, 2H), 2.90-2.87 (m, 2H), 2.52 (s, 6H). LCMS (ESI+) [M+H]/z Calc'd 392, found 392. Anal. (C₂₂H₂₁N₃O₂S.0.45CH₂Cl₂) C, H, N.

### Example 42

### Preparation of 6-(Thieno [3,2-d]pyrimidin-4-yloxy)-naphthalene-1-carboxylic acid (3-morpholin-4-yl-propyl)-amide

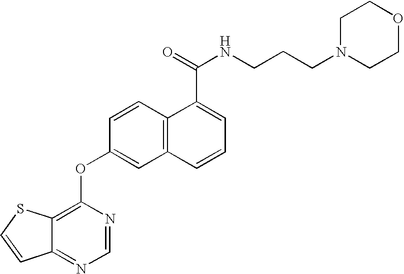

A. Preparation of Intermediate 42a: 6-(Thieno[3,2-d]pyrimidin-4-yloxy)-naphthalene-1-carboxylic acid

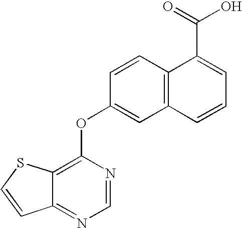

Intermediate 42a was prepared from 4-chloro-thieno[3,2-d]pyrimidine and 6-hydroxy-1-naphthoic acid in a manner as described previously in Method C. ¹H NMR (300 MHz, DMSO-d₆) δ 8.97 (d, 1H, J=9.42 Hz), 8.70 (s, 1H), 8.50 (d, 1H, J=5.28 Hz), 8.16 (d, 2H, J=8.85 Hz), 7.75-7.60 (m, 3H). LCMS (ESI+) [M+H]/z Calc'd 323, found 323. Anal. (C₁₇H₁₀N₂O₃S.0.5H₂O)C, H, N.

B. Preparation of Title Compound

The compound of Example 42 was prepared by the reaction of 6-(thieno[3,2-d]pyrimidin-4-yloxy)-naphthalene-1-carboxylic acid (42a) with 3-morpholin-4-yl-propylamine in a manner as described previously in Method A to give the title compound. ¹H NMR (300 MHz, CD₃OD) δ 8.54 (s, 1H), 8.27-8.17 (m, 2H), 7.91 (d, 1H, J=7.92 Hz), 7.77 (d, 1H, J=2.26 Hz), 7.60-7.53 (m, 1H), 7.52-7.38 (m, 3H), 3.64-3.52 (m, 4H), 3.49-3.39 (m, 2H), 2.57-2.43 (m, 6H), 1.91-1.77 (m, 2H). LCMS (ESI+) [M+H]/z Calc'd 449, found 449. Anal. (C₂₄H₂₄N₄O₃S)C, H, N.

### Example 43

### Preparation of 6-(7-Methoxy-quinolin-4-yloxy)-naphthalene-1-carboxylic Acid Methylamide

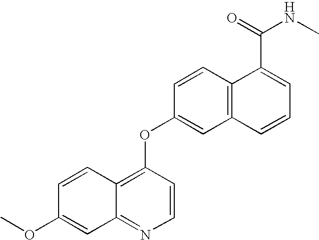

### A. Preparation of Intermediate 43a: 6-(7-Methoxy-quinolin-4-yloxy)-naphthalene-1-carboxylic Acid

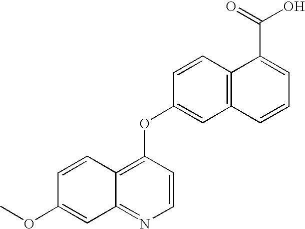

A mixture of 4-chloro-7-methoxy-quinoline (preparation described below) (200 mg, 1.036 mmol), 6-hydroxy-1-naphthoic acid (200 mg, 1.062 mmol), and Cs₂CO₃ (658 mg, 2.01 mmol) in 2 mL of DMSO was heated at 120° C. in a seal tube for 5 hours, and cooled to room temperature. EtOAc and water were added. The aqueous layer was acidified with 1N HCl until a precipitate was formed. The solid was filtered and washed with water, and dried in vacuum oven at 60° C. overnight. The title compound (210 mg) was obtained as a brown solid. ¹H NMR (300 MHz, DMSO-d₆) δ 9.03 (d, 1H, J=9.23 Hz), 8.64 (d, 1H, J=5.08 Hz), 8.24 (d, 1H, J=9.05 Hz), 8.17 (d, 2H, J=7.73 Hz), 7.91 (s, 1H), 7.72-7.57 (m, 2H), 7.45 (d, 1H, J=1.69 Hz), 7.36-7.30 (m, 1H), 6.61 (d, 1H, J=5.09 Hz), 3.95 (s, 3H). LCMS (ESI+) [M+H]/z Calc'd 346, found 346.
B. Preparation of Intermediate 4-chloro-7-methoxyquinoline (F):

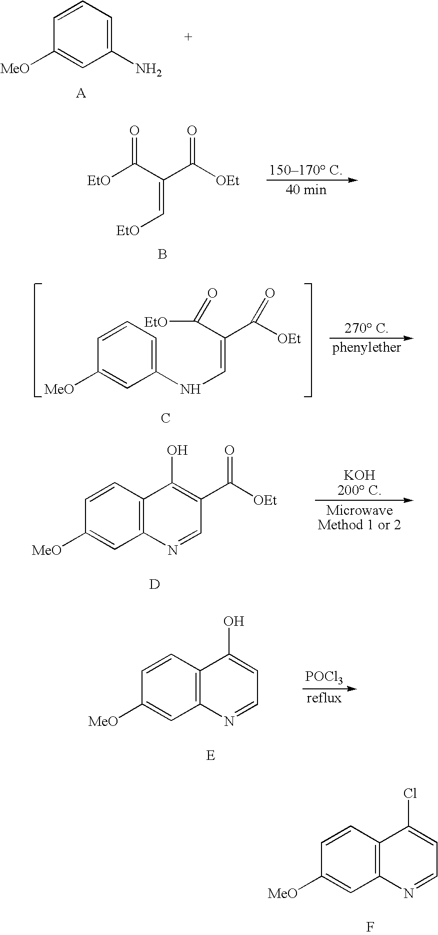

A mixture of 3-methoxyaniline A (25 g, 204 mmol) and diethyl (ethoxymethylene)malonate B (44.2 g, 204 mmol) was placed in a 250 mL round bottom flask and heated in an oil bath. When the temperature of the oil bath reached ˜135° C., EtOH was generated and collected with a condenser. The reaction was heated at 150° C. for 40 minutes to give C. ¹H NMR (300 MHz, DMSO-d₆) d 10.67 (d, J=13.75 Hz, 1H), 8.41 (d, J=13.94 Hz, 1H), 7.30 (t, J=8.10 Hz, 1H), 6.99 (d, J=2.07 Hz, 1H), 6.93 (dd, J=8.01, 1.79 Hz, 1H), 6.74 (dd, J=8.10, 2.26 Hz, 1H), 4.17 (m, 4H), 3.78 (s, 3H), 1.24 (m, 6H). The reaction flask was removed from the oil bath. Phenyl ether (about two times the volume of the reaction mixture) was added into the flask. The reaction flask was placed in the oil bath, which was preheated to 270° C. The reaction mixture was stirred and heated to 272° C. for 15 minutes (the temperature of inside the flask was 241° C.). The reaction flask was removed from heating and reaction mixture was slowly poured into hexane (1 L). Ethyl 4-hydroxy-7-methoxyquinoline-3-carboxylate D was precipitated and collected by filtration. The compound washed by hexane (to remove phenyl ether) and dried (28.4 g). Compound D: ¹H NMR (300 MHz, DMSO-d₆) d 12.10 (s, 1H), 8.49 (d, J=6.59 Hz, 1H), 8.05 (d, J=9.61 Hz, 1H), 7.00 (m, 2H), 4.20 (m, 2H), 3.87 (m, 3H), 1.26 (m, 3H).

Method 1: A solution of ethyl 4-hydroxy-7-methoxyquinoline-3-carboxylate D (5 g) and KOH (3 eq) in 60 mL of H₂O/HO(CH₂)₂OH (1:1) was placed in a sealed vessel (XP-500 Plus vessel). The reaction was heated by microwave (MARS 5 Microwave System) at 200° C., under 220-240 psi pressure for 30 minutes. The reaction mixture was cooled to room temperature and poured into H₂O (100 mL). The solution was acidified with 2 N HCl to pH˜6, saturated with NaCl and extracted with THF (3×200 mL). The combined oil layer was washed with brine and concentrated to give compound E (>80% yield). Compound E: ¹H NMR (300 MHz, DMSO-d₆) d 11.55 (s, 1H), 7.81 (dd, J=7.25, 5.93 Hz, 1H), 6.91 (m, 2H), 5.94 (d, J=7.54 Hz, 1H), 3.85 (m, 3H).

Method 2: A solution ethyl 4-hydroxy-7-methoxyquinoline-3-carboxylate D (5 g) and KOH (3 eq) in 60 mL of H₂O/EtOH (1:1) was placed in a sealed vessel (XP-500 Plus vessel) and heated by microwave (MARS 5 Microwave System) at 180° C., under 260-280 psi pressure for 50 minutes. The reaction mixture was cooled to room temperature and poured into H₂O (100 mL). The solution was acidified with 2 N HCl to pH˜6, saturated with NaCl and extracted with THF (3×200 mL). The combined oil layers were washed with brine and concentrated to give compound E (>80% yield).

7-Methoxyquinolin-4-ol E (7.4 g) was dissolved in neat POCl₃ (20 mL). The solution was heated to reflux for 2 hours. The excess amount of POCl₃ was removed by evaporation under vacuum. The residue was basified with NH₄OH to pH 8˜9 and extracted with EtOAc. The organic layer was concentrated. The residue was purified by column chromatography using 3:1 to 1:1 Hexane/EtOAc to give 4-chloro-7-methoxyquinoline E (6.5 g). Compound F: ¹H NMR (300 MHz, DMSO-d₆) d 8.85 (d, J=5.09 Hz, 1H), 8.15 (d, J=9.23 Hz, 1H), 7.70 (d, J=4.90 Hz, 1H), 7.54 (d, J=2.45 Hz, 1H), 7.47 (dd, J=9.23, 2.45 Hz, 1H), 3.97 (m, 3H).

C. Preparation of Title Compound

To a suspension of 6-(7-methoxy-quinolin-4-yloxy)-naphthalene-1-carboxylic acid (43a) (120 mg, 0.378 mmol) in CH₂Cl₂ (4 mL) was added 2.0 M oxalyl chloride in CH₂Cl₂ (0.57 mL, 1.134 mmol), followed by 2 drops of DMF. The mixture was stirred at ambient temperature for one hour, concentrated in vacuo, and further dried under high vacuum. The residue was dissolved in CH₂Cl₂ (4 mL), and to this solution was added 2.0 M methylamine in THF (1.04 ml, 2.08 mmol). The mixture was stirred at ambient temperature overnight and concentrated in vacuo. The residue was purified by flash column chromatography eluting with EtOAc:CHCl₃:MeOH (1:1:0.02) to render 111 mg white solid as the title compound. ¹H NMR (300 MHz, CD₃OD) δ 8.57 (d, 1H, J=5.28 Hz), 8.45 (d, 1H, J=9.04 Hz), 8.25 (d, 1H, J=9.04 Hz), 7.88 (d, 1H, J=8.10 Hz), 7.63-7.55 (m, 2H), 7.48 (d, 1H, J=8.10 Hz), 7.44 (d, 1H, J=2.64 Hz), 7.42-7.36 (m, 1H), 7.21 (d, 1H, J=2.45 Hz), 6.47 (d, 1H, J=5.28 Hz), 3.97 (s, 3H), 3.10 (s, 3H). LCMS (ESI+) [M+H]/z Calc'd 359, found 359. Anal. (C₂₂H₁₈N₂O₃) C, H, N.

### Example 44

### Preparation of 6-(7-Hydroxy-quinolin-4-yloxy)-naphthalene-1-carboxylic Acid Methylamide

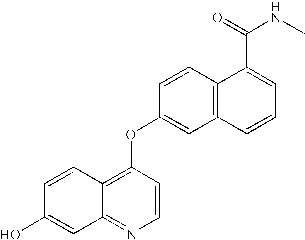

To a solution of 6-(7-methoxy-quinolin-4-yloxy)-naphthalene-1-carboxylic acid methylamide (Example 43) (100 mg, 0.279 mmol) in 3 mL of CH₂Cl₂ was added 1.0 M BBr₃ (0.84 mL, 0.837 mmol), and the mixture was stirred at room temperature overnight. Additional 1.0 M BBr₃ (0.84 ml, 0.837 mmol) was added, and the mixture was stirred for another 24 hours. The reaction was quenched with MeOH, and neutralized with concentrated NH₄OH until pH˜7. The resulting mixture was stirred at room temperature for one hour. Water was added, and extracted with CH₂Cl₂ for three times. The combined organic phase was dried over Na₂SO₄, concentrated in vacuo, and purified by reverse phase HPLC to give 92 mg of the title compound as yellow solid. ¹H NMR (300 MHz, CD₃OD) δ 8.34 (d, 1H, J=5.46 Hz), 8.27 (d, 1H, J=9.23 Hz), 8.18 (d, 1H, J=9.04 Hz), 7.88 (d, 1H, J=7.91 Hz), 7.64 (d, 1H, J=2.45 Hz), 7.56-7.43 (m, 2H), 7.38-7.32 (m, 1H), 7.18 (d, 1H, J=2.26 Hz), 7.13-7.09 (m, 1H), 6.39 (d, 1H, J=5.46 Hz), 2.90 (s, 3H). LCMS (ESI+) [M+H]/z Calc'd 345, found 345. Anal. (C₂₁H₁₆N₂O₃.0.5EtOAc.0.1CHCl₃) C, H, N.

### Example 45

### Preparation of 6-[7-(2-Morpholin-4-yl-ethoxy)-quinolin-4-yloxy]-naphthalene-1-carboxylic Acid Methylamide

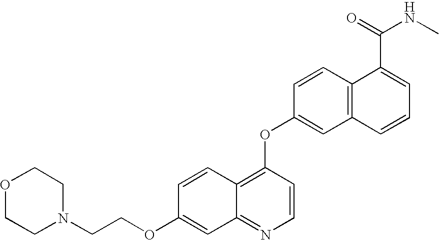

To a solution of 4-(2-chloroethyl)morpholine.HCl (32 mg, 0.163 mmol) in 1 mL of DMSO was added Cs₂CO₃ (133 mg, 0.405 mmol). The mixture was stirred at room temperature for one hour and to this was added 6-(7-hydroxy-quinolin-4-yloxy)-naphthalene-1-carboxylic acid methylamide (Example 44) (40 mg, 0.116 mmol) in 0.5 mL of DMSO. The mixture was heated at 120° C. for 2 hours, and cooled to room temperature. The residue was purified by reversed phase HPLC eluting with 20-60% acetonitrile in water to give 25 mg of the title compound as a white solid. ¹H NMR (300 MHz, CD₃OD) δ 8.44 (d, 1H, J=5.28 Hz), 8.28 (d, 1H, J=9.23 Hz), 8.24 (d, 1H, J=9.23 Hz), 7.88 (d, 1H, J=8.10 Hz), 7.66 (d, 1H, J=2.26 Hz), 7.67-7.43 (m, 2H), 7.39-7.33 (m, 1H), 7.30 (d, 1H, J=2.26 Hz), 7.27-7.20 (m, 1H), 6.47 (d, 1H, J=5.46 Hz), 4.28-4.20 (m, 2H), 3.66-3.63 (m, 4H), 2.33-2.30 (m, 2H), 2.58-2.53 (m, 4H). LCMS (ESI+) [M+H]/z Calc'd 458, found 458. Anal. (C₂₇H₂₇N₃O₄.0.6CH₃COOH)C, H, N.

### Example 46

### Preparation of 6-[7-(2-Piperidin-1-yl-ethoxy)-quinolin-4-yloxy]-naphthalene-1-carboxylic Acid Methylamide

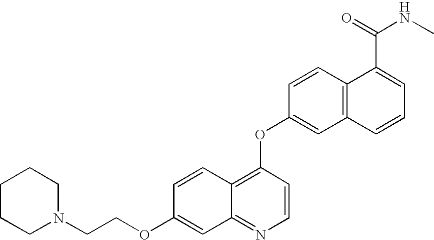

The title compound was prepared by the reaction of 6-(7-hydroxy-quinolin-4-yloxy)-naphthalene-1-carboxylic acid methylamide (Example 44) with 1-(2-chloro-ethyl)-piperidine and Cs₂CO₃ in a manner as described previously for the preparation of the compound of Example 45. ¹H NMR (300 MHz, CD₃OD) δ 8.25 (d, 1H, J=5.28 Hz), 8.30-8.22 (m, 2H), 7.88 (d, 1H, J=8.10 Hz), 7.66 (d, 1H, J=1.69 Hz), 7.57-7.42 (m, 2H), 7.39-7.21 (m, 3H), 6.47 (d, 1H, J=5.47 Hz), 4.37-4.27 (m, 2H), 3.12-3.05 (m, 2H), 2.90 (s, 3H), 2.87-2.75 (m, 4H), 1.71-1.60 (m, 4H), 1.53-1.42 (m, 2H). LCMS (ESI+) [M+H]/z Calc'd 456, found 456. Anal. (C₂₈H₂₉N₃O₃.0.5H₂O.0.6CH₃COOH)C, H, N.

### Example 47

### Preparation of 6-(7-Methoxy-quinolin-4-yloxy)-naphthalene-1-carboxylic Acid Butylamide

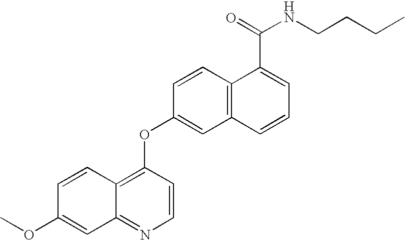

The compound of Example 46 was prepared by the coupling of 6-(7-methoxy-quinolin-4-yloxy)-naphthalene-1-carboxylic acid (43a) and butylamine in a manner as described previously for the preparation of the compound of Example 43. ¹H NMR (300 MHz, CD₃OD) δ 8.46 (d, 1H, J=5.48 Hz), 8.30-8.21 (m, 2H), 7.90 (d, 1H, J=7.91 Hz), 7.68 (d, 1H, J=2.44 Hz), 7.56-7.46 (m, 2H), 7.41-7.35 (m, 1H), 7.29 (d, 1H, J=2.24 Hz), 7.25-7.19 (m, 1H), 6.48 (d, 1H, J=5.46 Hz), 3.90 (s, 3H), 3.43-3.36 (m, 2H), 1.65-1.54 (m, 2H), 1.47-1.35 (m, 2H), 0.97-0.89 (m, 3H). LCMS (ESI+) [M+H]/z Calc'd 401, found 401. Anal. (C₂₅H₂₄N₂O₃) C, H, N.

### Example 48

### Preparation of 6-(7-Hydroxy-quinolin-4-yloxy)-naphthalene-1-carboxylic Acid Butylamide

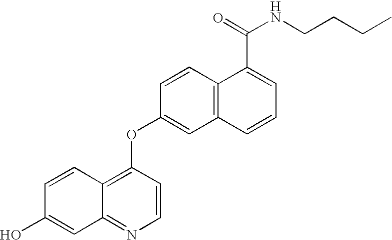

The compound of Example 48 was prepared from Example 47 and BBr₃ in a manner as described previously for the preparation of the compound of Example 44. ¹H NMR (300 MHz, CD₃OD) □ 8.40 (d, 1H, J=5.27 Hz), 8.26 (d, 1H, J=9.23 Hz), 8.20 (d, 1H, J=9.04 Hz), 7.90 (d, 1H, J=7.92 Hz), 7.67 (d, 1H, J=2.45 Hz), 7.57-7.45 (m, 2H), 7.41-7.35 (m, 1H), 7.21 (d, 1H, J=2.26 Hz), 7.18-7.12 (m, 1H), 6.42 (d, 1H, J=5.28 Hz), 5.41 (s, 1H), 3.43-3.36 (m, 2H), 1.65-1.54 (m, 2H), 1.47-1.35 (m, 2H), 0.97-0.89 (m, 3H). LCMS (ESI+) [M+H]/z Calc'd 387, found 387. Anal. (C₂₄H₂₂N₂O₃.0.06CHCl₃) C, H, N.

### Example 49

### Preparation of 6-[7-(2-Pyrrolidin-1-yl-ethoxy)-quinolin-4-yloxy]-naphthalene-1-carboxylic Acid Butylamide

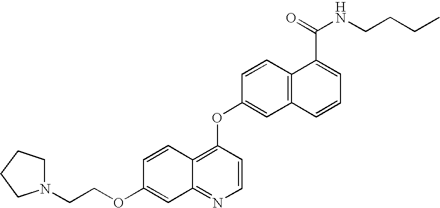

The compound of Example 49 was prepared from the compound of Example 48 and 1-(2-chloro-ethyl)-pyrrolidine in a manner as described previously for the preparation of the compound of Example 45. ¹H NMR (300 MHz, CD₃OD) δ 8.25 (d, 1H, J=5.46 Hz), 8.29-8.19 (m, 2H), 7.85 (d, 1H, J=8.10 Hz), 7.63 (d, 1H, J=2.07 Hz), 7.56-7.42 (m, 2H), 7.37-7.18 (m, 3H), 6.45 (d, 1H, J=5.47 Hz), 4.33-4.20 (m, 2H), 3.42-3.30 (m, 2H), 3.17-3.06 (m, 2H), 2.98-2.74 (m, 4H), 1.92-1.78 (m, 4H), 1.61-1.49 (m, 2H), 1.43-1.31 (m, 2H), 0.94-0.85 (m, 3H). LCMS (ESI+) [M+H]/z Calc'd 484, found 484. Anal. (C₃₀H₃₃N₃O₃.0.25CH₂Cl₂) C, H, N.

### Example 50

### Preparation of 6-(7-Methoxy-quinolin-4-yloxy)-naphthalene-1-carboxylic Acid Isopropylamide

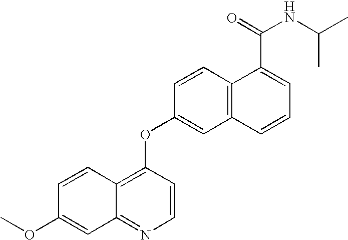

The compound of Example 50 was prepared by the coupling of intermediate 43a and isopropylamine in a manner as described previously in Method A to give the title compound. ¹H NMR (300 MHz, CD₃OD) δ 8.44 (d, 1H, J=5.47 Hz), 8.28-8.17 (m, 2H), 7.87 (d, 1H, J=7.73 Hz), 7.67 (d, 1H, J=2.45 Hz), 7.55-7.43 (m, 2H), 7.40-7.32 (m, 1H), 7.28 (d, 1H, J=2.45 Hz), 7.24-7.15 (m, 1H), 6.46 (d, 1H, J=5.46 Hz), 4.29-4.15 (m, 1H), 3.89 (s, 3H), 1.23 (s, 3H), 1.21 (s, 3H). LCMS (ESI+) [M+H]/z Calc'd 387, found387. Anal. (C₂₄H₂₂N₂O₃) C, H, N.

### Example 51

### Preparation of 6-(7-Methoxy-quinolin-4-yloxy)-naphthalene-1-carboxylic acid cyclopropylamide

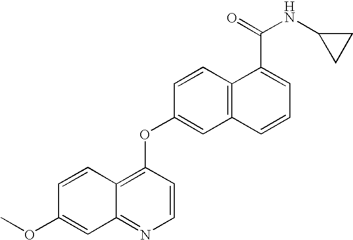

The compound of Example 51 was prepared by the coupling of intermediate 43a and cyclopropylamine in a manner as described previously in Method A to give the title compound. ¹H NMR (300 MHz, CD₃OD) δ 8.44 (d, 1H, J=5.28 Hz), 8.28 (d, 1H, J=9.04 Hz), 8.21 (d, 1H, J=9.21 Hz), 7.88 (d, 1H, J=7.16 Hz), 7.65 (d, 1H, J=2.26 Hz), 7.50-7.41 (m, 2H), 7.40-7.33 (m, 1H), 7.29 (d, 1H, J=2.45 Hz), 7.24-7.17 (m, 1H), 6.46 (d, 1H, J=5.28 Hz), 3.89 (s, 3H), 2.96-2.85 (m, 1H), 0.83-0.73 (m, 2H), 0.65-0.56 (m, 2H), LCMS (ESI+) [M+H]/z Calc'd 385, found 385. Anal. (C₂₄H₂₀N₂O₃.0.1CH₂Cl₂) C, H, N.

### Example 52

### Preparation of N-(2-Morpholin-4-ylethyl)-6-(thieno[3,2-d]pyrimidin-4-yloxy)-1-naphthamide

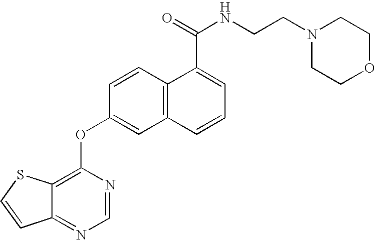

The compound of Example 52 was prepared by the reaction of 6-hydroxy-N-(2-morpholin-4-ylethyl)-1-naphthamide (12a) (100 mg, 0.33 mmol) with 4-chlorothieno[3,2-d]pyrimidine (57 mg, 0.33 mmol) and Cs₂CO₃ (346 mg, 1.05 mmol) in a manner as described previously in Method D to afford the title compound, 75 mg, 52%, as a white solid. HPLC: Rₜ 3.76 min (100% area). ¹H NMR (400 MHz, DMSO-d₆) δ 8.77 (1H, s), 8.90 (1H, t, J=4.2 Hz), 7.58 (1H, d, J=5.5 Hz), 8.09 (1H, q, J=4.8 Hz), 8.04 (1H, bs), 7.78 (1H, d, J=2.2 Hz), 7.68 (1H, d, J=4.1 Hz), 7.65 (1H, d, J=2.2 Hz), 3.67 (4H, t, J=4.0 Hz), 3.55 (2H, q, J=6.3 Hz), 3.55 (2H, q, J=6.3 Hz), 2.60-2.50 (4H, m). HRMS (ESI) C₂₃H₂₃N₄O₃S (M+H⁺) m/z: Calc. 435.1491, Found: 435.1513. Anal. (C₂₃H₂₂N₄O₃S.0.2H₂O) Calc'd: C, 63.05; H, 5.15; N, 12.79. Found: C, 63.09; H, 5.15; N, 12.67.

### Example 53

### Preparation of 6-[(6-Methylthieno[3,2-d]pyrimidin-4-yl)oxy]-N-(2-morpholin-4-ylethyl)-1-naphthamide

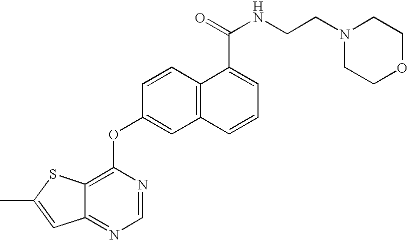

### A. Preparation of Intermediate 53a: 4-Chloro-6-methylthieno[3,2-d]pyrimidine

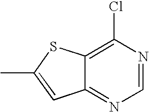

To a solution of 4-chlorothieno[3,2-d]pyrimidine (1.0 g, 5.86 mmol) in 10 mL of anhydrous THF cooled at −78° C. was added 1.6 M sec-butyl lithium in THF (4.06 mL, 6.45 mmol) dropwise over a period of 30 minutes, followed by addition of methyl iodide (0.80 mL, 11.72 mmol). The mixture was warmed to ambient temperature and stirred for 1 hour, the reaction was then quenched with 3.0N aqueous HCl. EtOAc (50 mL) was added, the mixture was washed with saturated aqueous NaHCO₃ solution (2×50 mL) and the organic layer was dried over Na₂SO₄ then concentrated to dryness. The residue was purified by flash chromatography eluting with Et₂O/EtOAc (9:1) to afford the title compound, 0.35 g, 33% yield, as a white solid. HPLC: R^{t} 3.79 min. (90% area). ¹H NMR (400 MHz, DMSO-d₆) δ 9.04 (1H, s), 7.58 (1H, s), 2.80 (3H, s). LCMS (APCI) (M+H⁺) m/z: 185.0.

B. Preparation of Title Compound

The compound of Example 53 was prepared by the reaction of 6-hydroxy-N-(2-morpholin-4-ylethyl)-1-naphthamide (12a) (110 mg, 0.37 mmol) with 4-chloro-6-methylthieno[3,2-d]pyrimidine (53a) (57 mg, 0.33 mmol) and Cs₂CO₃ (346 mg, 1.05 mmol) in a manner as described previously in Method D to afford the title compound, 120 mg, 82%, as a white solid. HPLC: Rₜ 3.84 min (100% area). ¹H NMR (400 MHz, DMSO-d₆) δ 8.70 (1H, s), 8.60 (1H, s), 8.46 (1H, d, J=9.1 Hz), 8.08 (1H, s), 8.01 (1H, s), 7.67-7.63 (3H, m), 3.67 (3H, s), 3.55-3.50 (2H, m), 2.79 (3H, s), 2.60-2.50 (8H, m). HRMS (ESI) C₂₄H₂₅N₄O₃S (M+H⁺) m/z: Calc. 449.647, Found: 449.1613. Anal. (C₂₃H₂₂N₄O₃S_{0.2} EtOAc) Calc'd: C, 63.90; H, 5.54; N, 12.02. Found: C, 63.52; H, 5.35; N, 12.25.

Biological Testing—Enzyme Assays

The stimulation of cell proliferation by growth factors such as VEFG, FGF, and others is dependent upon their induction of autophosphorylation of each of their respective receptor's tyrosine kinases. Therefore, the ability of a protein kinase inhibitor to block cellular proliferation induced by these growth factors is directly correlated with its ability to block receptor autophosphorylation. To measure the protein kinase inhibition activity of the compounds, the following constructs were devised.

(i) VEGF-R2 Construct for Assay:

This construct determines the ability of a test compound to inhibit tyrosine kinase activity. A construct (VEGF-R2D50) of the cytosolic domain of human vascular endothelial growth factor receptor 2 (VEGF-R2) lacking the 50 central residues of the 68 residues of the kinase insert domain was expressed in a baculovirus/insect cell system. Of the 1356 residues of full-length VEGF-R2, VEGF-R2D50 contains residues 806-939 and 990-1171, and also one point mutation (E990V) within the kinase insert domain relative to wild-type VEGF-R2. Autophosphorylation of the purified construct was performed by incubation of the enzyme at a concentration of 4 mM in the presence of 3 mM ATP and 40 mM MgCl₂ in 100 mM HEPES, pH 7.5, containing 5% glycerol and 5 mM DTT, at 4° C. for 2 h. After autophosphorylation, this construct has been shown to possess catalytic activity essentially equivalent to the wild-type autophosphorylated kinase domain construct. See Parast et al., Biochemistry, 37, 16788-16801 (1998).

(ii) FGF-R1 Construct for Assay:

The intracellular kinase domain of human FGF-R1 was expressed using the baculovirus vector expression system starting from the endogenous methionine residue 456 to glutamate 766, according to the residue numbering system of Mohammadi et al., Mol. Cell. Biol., 16, 977-989 (1996). In addition, the construct also has the following 3 amino acid substitutions: L457V, C488A, and C584S.

### Example A

VEGF-R2 Assay: Coupled Spectrophotometric (FLVK-P) Assay

The production of ADP from ATP that accompanies phosphoryl transfer was coupled to oxidation of NADH using phosphoenolpyruvate (PEP) and a system having pyruvate kinase (PK) and lactic dehydrogenase (LDH). The oxidation of NADH was monitored by following the decrease of absorbance at 340 nm (e₃₄₀=6.22 cm⁻¹ mM⁻¹) using a Beckman DU 650 spectrophotometer. Assay conditions for phosphorylated VEGF-R2D50 (indicated as FLVK-P in the tables below) were the following: 1 mM PEP; 250 mM NADH; 50 units of LDH/mL; 20 units of PK/mL; 5 mM DTT; 5.1 mM poly(E₄Y₁); 1 mM ATP; and 25 mM MgCl₂ in 200 mM HEPES, pH 7.5. Assay conditions for unphosphorylated VEGF-R2D50 (indicated as FLVK in the tables) were the following: 1 mM PEP; 250 mM NADH; 50 units of LDH/mL; 20 units of PK/mL; 5 mM DTT; 20 mM poly(E₄Y₁); 3 mM ATP; and 60 mM MgCl₂ and 2 mM MnCl₂ in 200 mM HEPES, pH 7.5. Assays were initiated with 5 to 40 nM of enzyme. Kᵢ values were determined by measuring enzyme activity in the presence of varying concentrations of test compounds. The percent inhibition at 50 nm (% inhibition @ 50 nm) was determined by linear least-squares regression analysis of absorbance as a function of time. The binding inhibitions were fitted to equation as described by Morrison. The data were analyzed using Enzyme Kinetic and Kaleidagraph software.

### Example B

FGF-R Assay

The spectrophotometric assay was carried out as described above for VEGF-R2, except for the following changes in concentration: FGF-R=50 nM, ATP=2 mM, and poly(E4Y1)=15 mM.

### Example C

HUVEC+VEGF Proliferation Assay

This assay determines the ability of a test compound to inhibit the growth factor-stimulated proliferation of human umbilical vein endothelial cells (“HUVEC”). HUVEC cells (passage 3-4, Clonetics, Corp.) were thawed into EGM2 culture medium (Clonetics Corp) in T75 flasks. Fresh EGM2 medium was added to the flasks 24 hours later. Four or five days later, cells were exposed to another culture medium (F12K medium supplemented with 10% fetal bovine serum (FBS), 60 mg/mL endothelial cell growth supplement (ECGS), and 0.1 mg/mL heparin). Exponentially-growing HUVEC cells were used in experiments thereafter. Ten to twelve thousand HUVEC cells were plated in 96-well dishes in 100 ml of rich, culture medium (described above). The cells were allowed to attach for 24 hours in this medium. The medium was then removed by aspiration and 105 ml of starvation media (F12K+1% FBS) was added to each well. After 24 hours, 15 ml of test agent dissolved in 1% DMSO in starvation medium or this vehicle alone was added into each treatment well; the final DMSO concentration was 0.1%. One hour later, 30 ml of VEGF (30 ng/mL) in starvation media was added to all wells except those containing untreated controls; the final VEGF concentration was 6 ng/mL. Cellular proliferation was quantified 72 hours later by MTT dye reduction, at which time cells were exposed for 4 hours MTT (Promega Corp.). Dye reduction was stopped by addition of a stop solution (Promega Corp.) and absorbance at 595 nm was determined on a 96-well spectrophotometer plate reader.

### Example D

Mouse PK Assay

The pharmacokinetics (e.g., absorption and elimination) of drugs in mice were analyzed using the following experiment. Test compounds were formulated as a suspension in a 30:70 (PEG 400: acidified H₂O) vehicle. This solution was administered orally (p.o.) and intraperitoneally (i.p.) at 50 mg/kg to two distinct groups (n=4) of B6 female mice. Blood samples were collected via an orbital bleed at time points: 0 hour (pre-dose), 0.5 hr, 1.0 hr, 2.0 hr, and 4.0 hr post dose. Plasma was obtained from each sample by centrifugation at 2500 rpm for 5 min. Test compound was extracted from the plasma by an organic protein precipitation method. For each time bleed, 50 μL of plasma was combined with 1.0 mL of acetonitrile, vortexed for 2 min. and then spun at 4000 rpm for 15 min. to precipitate the protein and extract out the test compound. Next, the acetonitrile supernatant (the extract containing test compound) was poured into new test tubes and evaporated on a hot plate (25° C.) under a steam of N₂ gas. To each tube containing the dried test compound extract, 125 μL of mobile phase (60:40, 0.025 M NH₄H₂PO₄+2.5 mL/L TEA:acetonitrile) was added. The test compound was resuspended in the mobile phase by vortexing and more protein was removed by centrifugation at 4000 rpm for 5 min. Each sample was poured into an HPLC vial for test compound analysis on an Hewlett Packard 1100 series HPLC with UV detection. From each sample, 95 μL was injected onto a Phenomenex-Prodigy reverse phase C-18, 150×3.2 mm column and eluted with a 45-50% acetonitrile gradient run over 10 min. Test-compound plasma concentrations (μg/mL) were determined by a comparison to standard curve (peak area vs. conc. μg/mL) using known concentrations of test compound extracted from plasma samples in the manner described above. Along with the standards and unknowns, three groups (n=4) of quality controls (0.25 μg/mL, 1.5 μg/mL, and 7.5 μg/mL) were run to insure the consistency of the analysis. The standard curve had an R₂>0.99 and the quality controls were all within 10% of their expected values. The quantitated test samples were plotted for visual display using Kalidagraph software and their pharmacokinetic parameters were determined using WIN NONLIN software.

### Example E

Human Liver Microsome (HLM) Assay

Compound metabolism in human liver microsomes was measured by LC-MS analytical assay procedures as follows. First, human liver microsomes (HLM) were thawed and diluted to 5 mg/mL with cold 100 mM potassium phosphate (KPO₄) buffer. Appropriate amounts of KPO₄ buffer, NADPH-regenerating solution (containing B-NADP, glucose-6-phosphate, glucose-6-phosphate dehydrogenase, and MgCl₂), and HLM were preincubated in 13×100 mm glass tubes at 37° C. for 10 min. (3 tubes per test compound—triplicate). Test compound (5 μM final) was added to each tube to initiate reaction and was mixed by gentle vortexing, followed by incubation at 37° C. At t=0, and 2 h, a 250-uL sample was removed from each incubation tube to separate 12×75 mm glass tubes containing 1 mL ice-cold acetonitrile with 0.05 μM reserpine. Samples were centrifuged at 4000 rpm for 20 min. to precipitate proteins and salt (Beckman Allegra 6KR, S/N ALK98D06, #634). Supernatant was transferred to new 12×75 mm glass tubes and evaporated by Speed-Vac centrifugal vacuum evaporator. Samples were reconstituted in 200 μL 0.1% formic acid/acetonitrile (90/10) and vortexed vigorously to dissolve. The samples were then transferred to separate polypropylene microcentrifuge tubes and centrifuged at 14000×g for 10 min. (Fisher Micro 14, S/N M0017580). For each replicate (#1-3) at each timepoint (0 and 2 h), an aliquot sample of each test compound was combined into a single HPLC vial insert (6 total samples) for LC-MS analysis, which is described below.

The combined compound samples were injected into the LC-MS system, composed of a Hewlett-Packard HP1100 diode array HPLC and a Micromass Quattro II triple quadruple mass spectrometer operating in positive electrospray SIR mode (programmed to scan specifically for the molecular ion of each test compound). Each test compound peak was integrated at each timepoint. For each compound, peak area at each timepoint (n=3) was averaged, and this mean peak area at 2 h was divided by the average peak area at time 0 hour to obtain the percent test compound remaining at 2 h.

### Example F

KDR (VEGFR2) Phosphorylation in PAE-KDR Cells Assay

This assay determines the ability of a test compound to inhibit the autophosphorylation of KDR in porcine aorta endothelial (PAE)-KDR cells. PAE cells that overexpress human KDR were used in this assay. The cells were cultured in Ham's F12 media supplemented with 10% fetal bovine serum (FBS) and 400 ug/mL G418. Thirty thousands cells were seeded into each well of a 96-well plate in 75 mL of growth media and allowed to attach for 6 hours at 37° C. Cells were then exposed to the starvation media (Ham's F12 media supplemented with 0.1% FBS) for 16 hours. After the starvation period was over, 10 mL of test agent in 5% DMSO in starvation media were added to the test wells and 10 mL of the vehicle (5% DMSO in starvation media) were added into the control wells. The final DMSO concentration in each well was 0.5%. Plates were incubated at 37° C. for 1 hour and the cells were then stimulated with 500 ng/ml VEGF (commercially available from R & D System) in the presence of 2 mM Na₃VO₄ for 8 minutes. The cells were washed once with 1 mm Na₃VO₄ in HBSS and lysed by adding 50 mL per well of lysis buffer. One hundred mL of dilution buffer were then added to each well and the diluted cell lysate was transferred to a 96-well goat ant-rabbit coated plate (commercially available from Pierce) which was pre-coated with Rabbit anti Human Anti-flk-1 C-20 antibody (commercially available from Santa Cruz). The plates were incubated at room temperature for 2 hours and washed seven times with 1% Tween 20 in PBS. HRP-PY20 (commercially available from Santa Cruz) was diluted and added to the plate for a 30-minute incubation. Plates were then washed again and TMB peroxidase substrate (commercially available from Kirkegaard & Perry) was added for a 10-minute incubation. One hundred mL of 0.09N H₂SO₄ was added to each well of the 96-well plates to stop the reaction. Phosphorylation status was assessed by spectrophotometer reading at 450 nm. IC₅₀ values were calculated by curve fitting using a four-parameter analysis.

### Example G

PAE-PDGFRb Phosphorylation in PAE-PDGFRB Cells Assay

This assay determines the ability of a test compound to inhibit the autophosphorylation of PDGFRb in porcine aorta endothelial (PAE)-PDGFRb cells. PAE cells that overexpress human PDGFRb were used in this assay. The cells were cultured in Ham's F12 media supplemented with 10% fetal bovine serum (FBS) and 400 ug/ml G418. Twenty thousands cells were seeded in each well of a 96-well plate in 50 mL of growth media and allowed to attach for 6 hours at 37° C. Cells were then exposed to the starvation media (Ham's F12 media supplemented with 0.1% FBS) for 16 hours. After the starvation period was over, 10 mL of test agent in 5% DMSO in starvation media were added to the test wells and 10 mL of the vehicle (5% DMSO in starvation media) were added into the control wells. The final DMSO concentration in each well was 0.5%. Plates were incubated at 37° C. for 1 hour and the cells were then stimulated with 1 mg/mL PDGF-BB (R & D System) in the presence of 2 mM Na₃VO₄ for 8 minutes. The cells were washed once with 1 mm Na₃VO₄ in HBSS and lysed by adding 50 mL per well of lysis buffer. One hundred mL of dilution buffer were then added to each well and the diluted cell lysate was transferred to a 96-well goat ant-rabbit coated plate (Pierce), which was pre-coated with Rabbit anti Human PDGFRb antibody (Santa Cruz). The plates were incubated at room temperature for 2 hours and washed seven times with 1% Tween 20 in PBS. HRP-PY20 (Santa Cruz) was diluted and added to the plate for a 30-minute incubation. Plates were then washed again and TMB peroxidase substrate (Kirkegaard & Perry) was added for a 10-minute incubation. One hundred mL of 0.09N H₂SO₄ was added into each well of the 96-well plate to stop the reaction. Phosphorylation status was assessed by spectrophotometer reading at 450 nm. IC₅₀ values were calculated by curve fitting using a four-parameter analysis.

The results of the testing of the compounds using various assays are summarized in Table 1 below, where activates are expressed as being in the range

TABLE 1

| | FLVK Ki (nM) | HUVEC + VEGF IC50 (nM) |
|---|---|---|
| | A = >10 | A = >10 |
| | B = 1-10 | B = 1-10 |
| | C = <1 | C = <1 |
| Example Number | NT = Not tested | NT = Not tested |
| 1 | C | C |
| 2 | NT | C |
| 3 | C | C |
| 4 | C | B |
| 5 | B | B |
| 6 | C | A |
| 7 | B | A |
| 8 | B | A |
| 9 | C | NT |
| 10 | C | NT |
| 11 | B | NT |
| 12 | B | A |
| 13 | C | B |
| 14 | C | B |
| 15 | B | A |
| 16 | NT | NT |
| 17 | NT | NT |
| 18 | NT | NT |
| 19 | NT | NT |
| 20 | NT | NT |
| 21 | NT | NT |
| 22 | NT | NT |
| 23 | NT | NT |
| 24 | NT | NT |
| 25 | NT | NT |
| 26 | NT | NT |
| 27 | NT | NT |
| 28 | NT | NT |
| 29 | NT | NT |
| 30 | C | C |
| 31 | C | B |
| 32 | C | B |
| 33 | NT | NT |
| 34 | C | B |
| 35 | C | B |
| 36 | A | NT |
| 37 | A | NT |
| 38 | NT | NT |
| 39 | B | B |
| 40 | NT | NT |
| 41 | A | NT |
| 42 | B | A |
| 43 | B | B |
| 44 | A | NT |
| 45 | C | C |
| 46 | B | C |
| 47 | NT | A |
| 48 | NT | B |
| 49 | B | B |
| 50 | B | A |
| 51 | C | B |
| 52 | A | NT |
| 53 | NT | NT |

Examples of Pharmaceutical Formulations

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The exemplary compounds described above may be formulated into pharmaceutical compositions according to the following general examples.

### Example I

### Parenteral Composition

To prepare a parenteral pharmaceutical composition suitable for administration by injection, 100 mg of a water-soluble salt of a compound of Formula I is dissolved in DMSO and then mixed with 10 mL of 0.9% sterile saline. The mixture is incorporated into a dosage unit form suitable for administration by injection.

### Example II

### Oral Composition

To prepare a pharmaceutical composition for oral delivery, 100 mg of a compound of Formula I is mixed with 750 mg of lactose. The mixture is incorporated into an oral dosage unit for, such as a hard gelatin capsule, which is suitable for oral administration.

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

1. A compound represented by Formula (I): 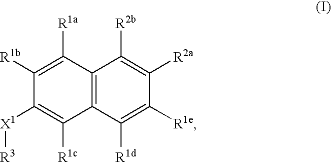
wherein
(a) one of R^{2a} and R^{2b} is —C(O)NHR⁴ and the other is R^{1f};
(b) each of R^{1a}, R^{1b}, R^{1c}, R^{1d}, R^{1e} and R^{1f} is independently selected from the group consisting of H, halogen, OH, NH₂, N₃, NO₂, (C₁-C₆)alkoxyl, (C₁-C₆)alkyl, (C₁-C₆)fluoroalkoxyl, and (C¹-C₆)fluoroalkyl;
(c) X¹ is O or S;
(d) R³ is either H or a moiety selected from the group consisting of —(CZ¹Z²)ⱼCN, —(CZ¹Z²)ⱼ—(C₃-C₈)cycloalkyl, —(CZ¹Z²)ⱼ—(C₅-C₈)cycloalkenyl, (C₂-C₆)alkenyl, (C₂-C₆)alkynyl, —(CZ¹Z²)ⱼ-aryl, —(CZ¹Z²)ⱼ-heterocyclyl, and (C₁-C₈)alkyl, where j is 0, 1, 2, or 3, and wherein when j is 2 or 3, each CZ¹Z² unit may be the same or different, and wherein Z¹ and Z² are independently selected from the group consisting of H, F, and (C₁-C₆)alkyl, or wherein Z¹ and Z² taken together can optionally form a carbocyclyl, or two Z¹ groups on adjacent atoms taken together can form a (C₃-C₈)carbocyclyl;
(e) R⁴ is either H or a moiety selected from the group consisting of —(CZ¹Z²)ⱼCN, —(CZ¹Z²)ⱼ—(C₃-C₈)cycloalkyl, —(CZ¹Z²)ⱼ—(C₅-C₈)cycloalkenyl, (C₂-C₆)alkenyl, (C₂-C₆)alkynyl, —(CZ¹Z²)ⱼ-aryl, —(CZ¹Z²)ⱼ-heterocyclyl, and (C₁-C₈)alkyl, where j is 0, 1, 2, or 3, and wherein when j is 2 or 3, each CZ¹Z² unit may be the same or different, and wherein Z¹ and Z² are independently selected from the group consisting of H, F, and (C₁-C₆)alkyl, or wherein Z¹ and Z² taken together can optionally form a (C₃-C₈)carbocyclyl, or two Z¹ groups on adjacent atoms taken together can form a (C₃-C₈)carbocycyl;
(f) wherein each R³ and R⁴ may be optionally substituted on any carbon atom containing a hydrogen atom, with 1-3 independently selected Y groups;
(g) each Y group:
(i) is independently selected from the group consisting of halogen, cyano, nitro, tetrazolyl, guanidino, amidino, methylguanidino, azido, C(O)Z³, —CF₃, —CF₂CF₃, —CH(CF₃)₂, —C(OH)(CF₃)₂, —OCF₃, —OCF₂H, —OCF₂CF₃, —OC(O)NH₂, —OC(O)NHZ³, —OC(O)NZ³Z⁴, —NHC(O)Z³, —NHC(O)NH₂, —NHC(O)NHZ³, —NHC(O)NZ³Z⁴, —C(O)OH, —C(O)OZ³, —C(O)NH₂, —C(O)NHZ³, —C(O)NZ³Z⁴, —P(O)₃H₂, —P(O)₃ (Z³)₂, —S(O)₃H, —S(O)ₘZ³, -Z³, —OZ³, —OH, —NH₂, —NHZ³, —NZ³Z⁴, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxyl, —(CZ⁵Z⁶)ᵣNH₂, —(CZ⁵Z⁶)ᵣNHZ₁, —(CZ⁵Z⁶)ᵣNZ³Z, —X²(CZ⁵Z⁶)ᵣ—(C₃-C₈)cycloalkyl, —X²(CZ⁵Z⁶)ᵣ—(C₅-C₈)cycloalkenyl, —X²(CZ⁵Z⁶)ᵣ-aryl, —X²(CZ⁵Z⁶)ᵣ-heterocyclyl and —S(O)ₘ(CF₂)_{q}CF₃, wherein m is 0, 1 or 2; q is 0, 1, 2, 3, 4, or 5; r is 1, 2, 3, or 4; X² is O, S, NH, —C(O)—, —C(O)NH—, or C(O)O—; Z³ and Z⁴ are independently selected from the group consisting of (C₁-C₁₂) alkyl, (C₂-C₁₂) alkenyl, (C₂-C₁₂) alkynyl, (C₃-C₈) cycloalkyl, (C₅-C₈) cycloalkenyl, (C₆-C₁₄) aryl, 5 to 14 membered heterocyclyl, 7 to 15 membered aryl alkyl, and 5 to 14 membered heteroaryl alkyl; and Z⁵ and Z⁶ are independently selected from the group consisting of hydrogen, fluorine, (C₁-C₁₂) alkyl, (C₆-C₁₄) aryl, 5 to 14 membered heteroaryl, 7 to 15 membered aryl alkyl, and 5 to 14 membered heteroaryl alkyl; or
(ii) any two Y groups attached to adjacent carbon atoms may be selected together to be —O[C(Z⁵)(Z⁶)]ᵣO—or —O[C(Z⁵)(Z⁶)]ᵣ₊₁—; or
(iii) any two Y groups attached to the same or adjacent carbon atoms may be selected together to form a carbocyclyl or heterocyclyl;
and wherein any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C¹-C₄)alkyl, (C₁-C₄)alkoxyl and —N[(C₁-C₄)alkyl][(C₁-C₄)alkyl];
or an N-oxide, pharmaceutically acceptable prodrug, pharmaceutically active metabolite, pharmaceutically acceptable salt, or pharmaceutically acceptable solvate thereof.

2. A compound according to claim 1, wherein R^{2a} is H and R^{2b} is —C(O)NHR⁴.

3. A compound according to claim 2, wherein X is O.

4. A compound according to claim 3, wherein one of R^{1a}, R^{1b}, R^{1c}, R^{1d}, R^{1e} and R^{1f} is selected from the group consisting of halogen, (C₁-C₆)alkoxyl, (C₁-C₆)alkyl, (C₁-C₆)fluoroalkoxyl, and (C₁-C₆)fluoroalkyl and the other five of R^{1a}, R^{1b}, R^{1c}, R^{1d}, R^{1e} and R^{1f} are H.

5. A compound according to claim 3, wherein only one of R^{1a}, R^{1b}, R^{1c}, R^{1d}, R^{1e} and R^{1f} is F and the other five of R^{1a}, R^{1b}, R^{1c}, R^{1d}, R^{1e} and R^{1f} are H.

6. A compound according to claim 5, wherein R³ is a moiety selected from the group consisting of —(C₃-C₈)cycloalkyl, —(C₅-C₈)cycloalkenyl, -aryl, and -heterocyclyl, optionally substituted with 1-3 independently selected Y groups.

7. A compound according to claim 2, wherein R³ is a moiety selected from the group consisting of -aryl and -heterocyclyl, optionally substituted with 1-3 independently selected Y groups.

8. A compound according to claim 5, wherein R³ is a moiety selected from the group consisting of -aryl and -heterocyclyl, optionally substituted with 1-3 independently selected Y groups.

9. A compound according to claim 8, wherein each Y group on R³ is selected from the group consisting of halogen, C(O)Z³, —OC(O)NHZ³, —OC(O)NZ³Z⁴, —NHC(O)Z³, —C(O)OZ³, —C(O)NHZ³, —C(O)NZ³ Z, -Z³, —OZ³, —NHZ³, —NZ³Z⁴, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxyl, —(CZ⁵Z⁶)ᵣNZ³Z¹, X²(CZ⁵Z⁶)ᵣ—(C₃-C₈)cycloalkyl, —X²(CZ⁵Z⁶)ᵣ—(C₅-C₈)cycloalkenyl, —X²(CZ⁵Z⁶)ᵣ-aryl, and —X²(CZ⁵Z⁶)ᵣ-heterocycyl, r is 1, 2, 3, or 4; X² is O, S, NH, —C(O)—, —C(O)NH—, or —C(O)O—; Z³ and Z⁴ are independently selected from the group consisting of (C₁-C₁₂) alkyl, (C₂-C₁₂) alkenyl, (C₂-C₁₂) alkynyl, (C₃-C₈) cycloalkyl, (C₅-C₆) cycloalkenyl, (C₆-C₁₄) aryl, 5 to 14 membered heterocyclyl, 7 to 15 membered aryl alkyl, and 5 to 14 membered heteroaryl alkyl; and Z⁵ and Z⁶ are independently selected from the group consisting of hydrogen, fluorine, (C₁-C₁₂) alkyl, (C₆-C₁₄) aryl, 5 to 14 membered heteroaryl, 7 to 15 membered aryl alkyl, and 5 to 14 membered heteroaryl alkyl.

10. A compound according to claim 8, wherein R⁴ is a moiety, optionally substituted with 1-3 independently selected Y groups, selected from the group consisting of —(CZ¹Z²)ⱼ—(C₃-C₈)cycloalkyl, (CZ¹Z²)ⱼ-aryl, —(CZ¹Z²)ⱼ-heterocyclyl, and (C₁-C₈)alkyl, wherein each Z¹ and Z² of each —CZ¹Z²— are independently H or F.

11. A compound according to claim 10, wherein R⁴ is a moiety, selected from the group consisting of —(CH₂)ⱼ—(C₃-C₈)cycloalkyl, —(CH₂)ⱼ-aryl, —(CH₂)ⱼ-heterocyclyl, and (C₁-C₈)alkyl, optionally substituted with 1-3 independently selected Y groups.

12. A compound according to claim 7, wherein R³ is selected from the group consisting of
(a) a 5-membered aromatic, monocyclic heterocyclyl having 1 to 4 heteroatoms selected from the group consisting of O, S, and N, optionally substituted with 1-3 independently selected Y groups; and
(b) a 6-membered aromatic, monocyclic heterocyclyl having 1 to 4 heteroatoms selected from the group consisting of O, S, and N, optionally substituted with 1-3 independently selected Y groups;
wherein heterocyclyl (a) and (b) may be optionally fused to another carbocyclyl or heterocyclyl to form a fused bicyclic ring structure.

13. A compound according to claim 11, wherein R³ is selected from the group consisting of
(a) a 5-membered aromatic, monocyclic heterocyclyl having 1 to 4 heteroatoms selected from the group consisting of O, S, and N, optionally substituted with 1-3 independently selected Y groups; and
(b) a 6-membered aromatic, monocyclic heterocyclyl having 1 to 4 heteroatoms selected from the group consisting of O, S, and N, optionally substituted with 1-3 independently selected Y groups;
wherein heterocyclyl (a) and (b) may be optionally fused to another carbocyclyl or heterocyclyl to form a fused bicyclic ring structure.

14. A compound according to claim 1, represented by Formula II: 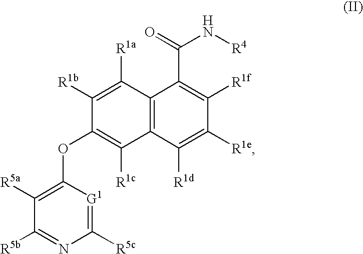
wherein
(a) G¹ is N or CR^{5d};
(b) each of R^{5a} and R^{5b} is independently H, halogen, or a moiety selected from the group consisting of —X³(CH₂)ₖ—(C₃-C₈)cycloalkyl, —X³(CH₂)ₖ—(C₅-C₈)cycloalkenyl, —X³(C₂-C₆)alkenyl, —X³(C₂-C₆)alkynyl, —X³(CH₂)ₖ-aryl, —X³(CH₂)ₖ-heterocyclyl, and —X³(C₁-C₈)alkyl, where k is 0, 1, 2, or 3, and wherein X³ is O, S, NH, —C(O)—, —C(O)NH—, or —C(O)O—; or optionally R^{5a} and R^{5b} taken together form a group, optionally substituted with 1-3 independently selected Y groups, selected from (C₃-C₈)cycloalkyl, (C₅-C₈)cycloalkenyl, (C₃-C₈)aryl, and (C₃-C₈)heterocyclyl; and
(c) each of R^{5c} and R^{5d} is independently H or halogen.

15. A compound according to claim 14, wherein one of R^{1a}, R^{1b}, R^{1c}, R^{1d}, R^{1e} and R^{1f} is selected from the group consisting of halogen, (C₁-C₆)alkoxyl, (C₁-C₆)alkyl, (C₁-C₆)fluoroalkoxyl, and (C¹-C₆)fluoroalkyl and the other five of R^{1a}, R^{1b}, R^{1c}, R^{1d}, R^{1e} and R^{1f} are H.

16. A compound according to claim 15, wherein one of R^{1a}, R^{1b}, R^{1c}, R^{1d}, R^{1e} and R^{1f} is F and the other five of R^{1a}, R^{1b}, R^{1c}, R^{1d}, R^{1e} and R^{1f} are H.

17. A compound according to claim 14, wherein R^{5a} and R^{5b} taken together form a group, optionally substituted with 1-3 independently selected Y groups, selected from (C₃-C₈)cycloalkyl, (C₅-C₈)cycloalkenyl, (C₃-C₈)aryl, and (C₃-C₈)heterocyclyl.

18. A compound according to claim 17, wherein R^{5a} and R^{5b} taken together form an aryl group, optionally substituted with 1-3 independently selected Y groups.

19. A compound according to claim 18, wherein each Y group on the aryl group formed from R^{5a} and R^{5b} is selected from the group consisting of halogen, —C(O)Z³, —OC(O)NHZ³, —OC(O)NZ³Z⁴, —NHC(O)Z³, —C(O)OZ³, —C(O)NHZ³, —C(O)NZ³Z⁴, -Z³, —OZ³, —NHZ³, —NZ³Z⁴, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxyl, —(CZ⁵Z⁶)ᵣNZ³Z⁴, —X²(CZ¹Z⁶)ᵣ—(C₅-C₈)cycloalkyl, —X²(CZ⁵Z⁶)ᵣ-aryl, and —X²(CZ⁵Z⁶)ᵣ-heterocyclyl.

20. A compound according to claim 19, wherein R⁴ is a moiety, optionally substituted with 1-3 independently selected Y groups, selected from the group consisting of —(CZ¹Z²)ⱼ—(C₃-C₈)cycloalkyl, (CZ¹Z²)ⱼ-aryl, —(CZ¹Z²)ⱼ-heterocyclyl, and (C₁-C₈)alkyl, wherein each Z¹ and Z² of each —CZ¹Z²-are independently H or F.

21. A compound according to claim 20, wherein R⁴ is a moiety, optionally substituted with 1-3 independently selected Y groups, selected from the group consisting of —(CH₂)ⱼ—(C₃-C₈)cycloalkyl, —(CH₂)ⱼ-aryl, —(CH₂)ⱼ-heterocyclyl, and (C₁-C₈)alkyl, where j is 0, 1, 2, or 3.

22. A compound according to claim 1 that is selected from the group consisting of:
6-[2-(1-Methyl-1H-imidazol-2-yl)-thieno[3,2-b]pyridin-7-yloxy]-naphthalene-1-carboxylic acid (2-pyrrolidin-1-yl-ethyl)-amide,
6-[7-(2-Piperidin-1-yl-ethoxy)-quinolin-4-yloxy]-naphthalene-1-carboxylic acid methylamide,
6-[7-(2-Morpholin-4-yl-ethoxy)-quinolin-4-yloxy]-naphthalene-1-carboxylic acid methylamide,
N-Methyl-6-{[2-({3-[(methylamino)methyl]pyrrolidin-1-yl}carbonyl)thieno[3,2-b]pyridin-7-yl]oxy}-1-naphthamide,
6-[2-(Azetidine-1-carbonyl)-thieno[3,2-b]pyridin-7-yloxy]-naphthalene-1-carboxylic acid (2-morpholin-4-yl-ethyl)-amide,
N-Cyclopropyl-6-[(2-{[(3R)-3-(dimethylamino)pyrrolidin-1-yl]carbonyl}thieno[3,2-b]pyridin-7-yl)oxy]-1-naphthamide,
6-[2-(1-Methyl-1H-imidazol-2-yl)-thieno[3,2-b]pyridin-7-yloxy]-naphthalene-1-carboxylic acid (3-morpholin-4-yl-propyl)-amide,
N-[3-(Dimethylamino)propyl]-N-methyl-7-({5-[(methylamino) carbonyl]-2-naphthyl}oxy)thieno[3,2-b]pyridine-2-carboxamide,
5-Fluoro-6-[(2-{[(3S)-3-methoxypyrrolidin-1-yl]carbonyl}thieno[3,2-b]pyridin-7-yl)oxy]-N-methyl-1-naphthamide,
6-(7-Methoxy-quinolin-4-yloxy)-naphthalene-1-carboxylic acid cyclopropylamide,
6-[7-(2-Pyrrolidin-1-yl-ethoxy)-quinolin-4-yloxy]-naphthalene-1-carboxylic acid butylamide,
6-{[2-(1-Methyl-1H-imidazol-2-yl)thieno[3,2-b]pyridin-7-yl]oxy}-N-(2-morpholin-4-ylethyl)-1-naphthamide,
6-[(2{[(3R)-3-Hydroxypyrrolidin-1-yl]carbonyl}thieno[3,2-b]pyridin-7-yl)oxy]-N-(2-morpholin-4-ylethyl)-1-naphthamide, and
6-[(2-{[(3S)-3-methoxypyrrolidin-1-yl]carbonyl}thieno[3,2-b]pyridin-7-yl)oxy]-N-methyl-1-naphthamide;
or an N-oxide, pharmaceutically acceptable prodrug, pharmaceutically active metabolite, pharmaceutically acceptable salt, or pharmaceutically acceptable solvate thereof.

23. A compound, N-oxide, pharmaceutically acceptable prodrug, pharmaceutically active metabolite, pharmaceutically acceptable salt, or pharmaceutically acceptable solvate according to claim 1 that is selected from the group consisting of: 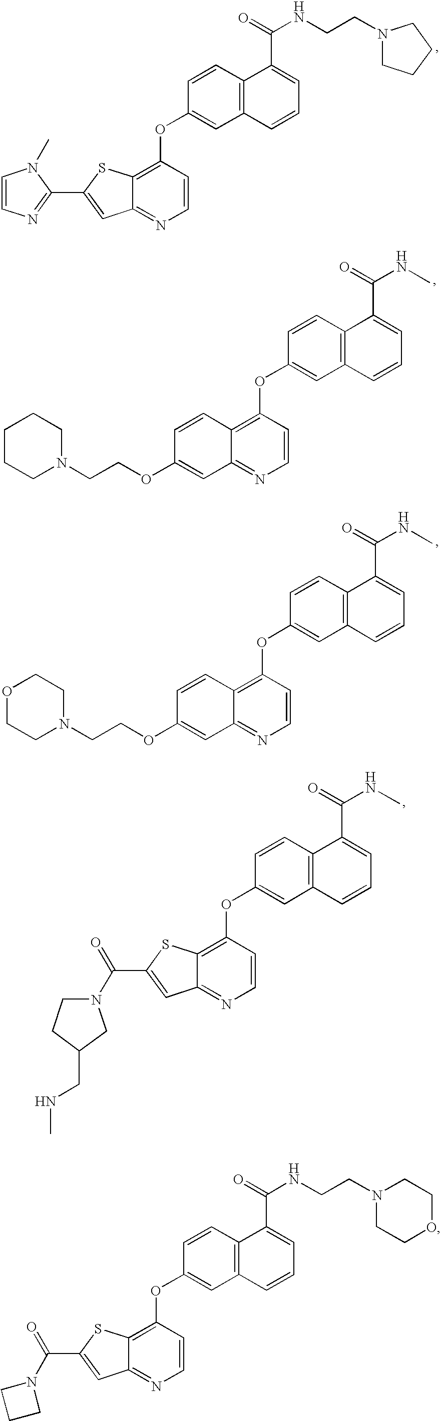 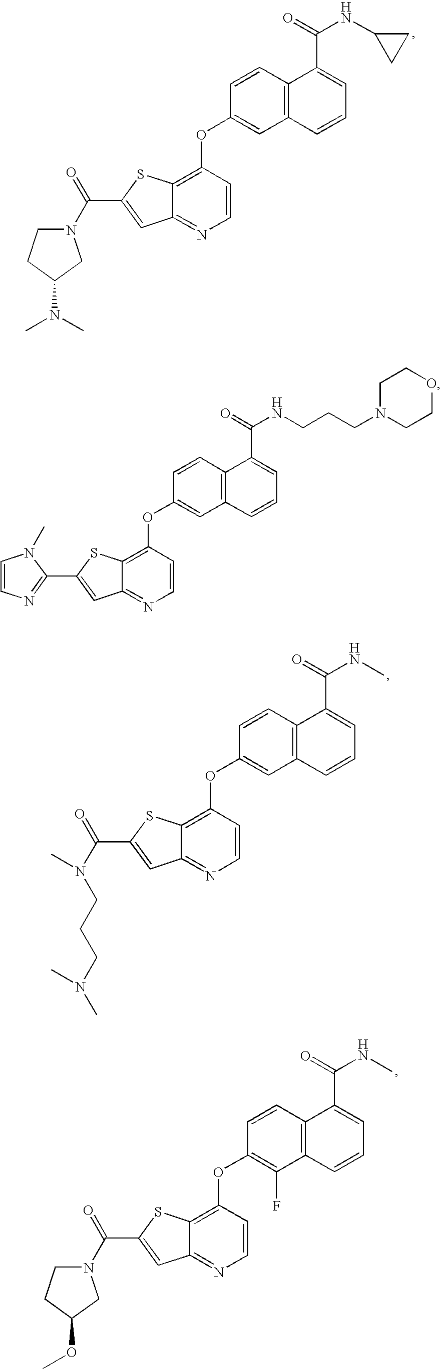 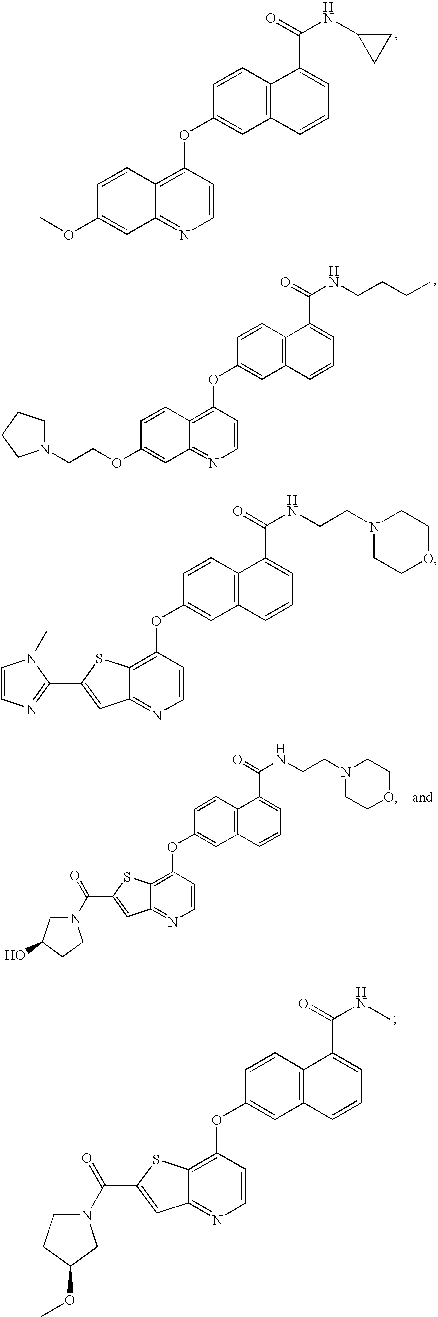
or an N-oxide, pharmaceutically acceptable prodrug, pharmaceutically active metabolite, pharmaceutically acceptable salt, or pharmaceutically acceptable solvate thereof.

24. A compound according to claim 1 wherein R³ is selected from the group consisting of 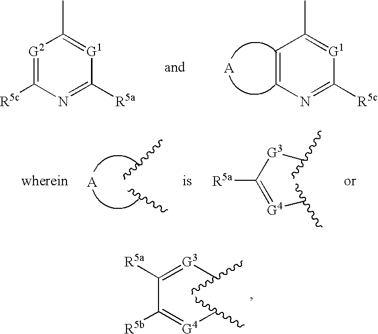
where
(a) G¹ and G² are N or C(R^{5d}), provided only one of G¹ and G² can be N;
(b) G³ and G⁴ are S, N or C(R^{5e}) provided only one of G³ and G⁴ can be S;
(c) each of R^{5a} and R^{5b} is independently H, halogen, or a moiety selected from the group consisting of —X³(CH₂)ₖ—(C₃-C₈)cycloalkyl, —X³(CH₂)ₖ—(C C8)cycloalkenyl, —X³(C₂-C₆)alkenyl, X³(C₂-C₆)alkynyl, —X³(CH₂)ₖ-aryl, —X³(CH₂)ₖ-heterocyclyl, and —X³(C₁-C₈)alkyl, where k is 0, 1, 2, or 3, and wherein X³ is O, S, NH, —C(O)—, —C(O)NH—, or —C(O)O—; or optionally R^{5a} and R^{5b} taken together form a group, optionally substituted with 1-3 independently selected Y groups, selected from (C₃-C₈)cycloalkyl, (C₅-C₈)cycloalkenyl, (C₃-C₈)aryl, and (C₃-C₈)heterocyclyl; and
(d) each of R^{5c}, and R^{5d} and R^{5e} is independently H or halogen.

25. A compound according to claim 24 wherein R³ is

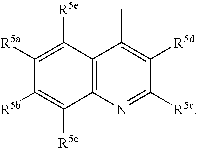

26. A compound according to claim 24 wherein R³ is

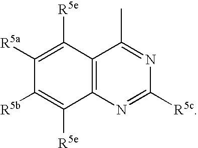

27. A compound according to claim 24 wherein R³ is

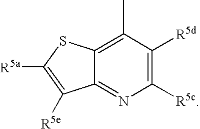

28. A compound according to claim 24 wherein R³ is

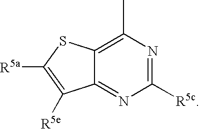

29. A compound according to claim 24 wherein R³ is

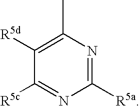

30. A pharmaceutical composition for the treatment of a hyperproliferative disorder in a mammal comprising a therapeutically effective amount of a compound, prodrug, metabolite, salt or solvate according to claim 1 and a pharmaceutically acceptable carrier.

31. A pharmaceutical composition according to claim 30, wherein said hyperproliferative disorder is cancer.

32. A pharmaceutical composition according to claim 31, wherein said cancer is brain, lung, kidney, renal, ovarian, ophthalmic, squamous cell, bladder, gastric, pancreatic, breast, head, neck, oesophageal, gynecological, prostate, colorectal or thyroid cancer.

33. A pharmaceutical composition according to claim 30, wherein said hyperproliferative disorder is noncancerous.

34. A pharmaceutical composition according to claim 33, wherein said hyperproliferative disorder is a benign hyperplasia of the skin or prostate.

35. A pharmaceutical composition for the treatment of a hyperproliferative disorder in a mammal comprising a therapeutically effective amount of a compound, prodrug, metabolite, salt or solvate according to claim 1 in combination with an anti-tumor agent selected from the group consisting of mitotic inhibitors, alkylating agents, anti-metabolites, intercalating antibiotics, enzymes, topoisomerase inhibitors, biological response modifiers, anti-hormones, and anti-androgens, and a pharmaceutically acceptable carrier.

36. A pharmaceutical composition for the treatment of pancreatitis or kidney disease in a mammal comprising a therapeutically effective amount of a compound, prodrug, metabolite, salt or solvate according to claim 1 and a pharmaceutically acceptable carrier.

37. A pharmaceutical composition for the prevention of blastocyte implantation in a mammal comprising a therapeutically effective amount of a compound, prodrug, metabolite, salt or solvate according to claim 1 and a pharmaceutically acceptable carrier.

38. A pharmaceutical composition for treating a disease related to vasculogenesis or angiogenesis in a mammal comprising a therapeutically effective amount of a compound, prodrug, metabolite, salt or solvate according to claim 1 and a pharmaceutically acceptable carrier.

39. A pharmaceutical composition according to claim 38 wherein said disease is selected from the group consisting of tumor angiogenesis, chronic inflammatory disease, atherosclerosis, skin diseases, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

40. A pharmaceutical composition for treating a disease related to vasculogenesis or angiogenesis in a mammal comprising a therapeutically effective amount of a compound, prodrug, metabolite, salt or solvate according to claim 1, a therapeutically effective amount of a compound, prodrug, metabolite, salt or solvate of an antihypertensive agent, and a pharmaceutically acceptable carrier.

41. A method of treating a hyperproliferative disorder in a mammal comprising administering to said mammal a therapeutically effective amount of a compound, prodrug, metabolite, salt or solvate according to claim 1.

42. A method according to claim 41 wherein said hyperproliferative disorder is cancer.

43. A method according to claim 42 wherein said cancer is brain, lung, ophthalmic, squamous cell, renal, kidney, ovarian, bladder, gastric, pancreatic, breast, head, neck, oesophageal, prostate, colorectal, gynecological or thyroid cancer.

44. A method according to claim 41 wherein said hyperproliferative disorder is noncancerous.

45. A method according to claim 44 wherein said hyperproliferative disorder is a benign hyperplasia of the skin or prostate.

46. A method for the treatment of a hyperproliferative disorder in a mammal comprising administering to said mammal a therapeutically effective amount of a compound, prodrug, metabolite, salt or solvate according to claim 1 in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

47. A method of treating pancreatitis or kidney disease in a mammal comprising administering to said mammal a therapeutically effective amount of a compound, prodrug, metabolite, salt or solvate according to claim 1.

48. A method of preventing blastocyte implantation in a mammal comprising administering to said mammal a therapeutically effective amount of a compound, prodrug, metabolite, salt or solvate according to claim 1.

49. A method for treating a disease related to vasculogenesis or angiogenesis in a mammal comprising administering to said mammal a therapeutically effective amount of a compound, prodrug, metabolite, salt or solvate according to claim 1.

50. A method according to claim 49 wherein said disease is selected from the group consisting of tumor angiogenesis, chronic inflammatory disease, atherosclerosis, skin diseases, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

51. A method for treating a disease related to vasculogenesis or angiogenesis in a mammal comprising administering to said mammal a therapeutically effective amount of a compound, prodrug, metabolite, salt or solvate according to claim 1 in conjunction with a therapeutically effective amount of an anti-hypertensive agent.

52. A method of producing a compound having the formula of claim 14, comprising:
(a) reacting a carboxylic acid having the structure 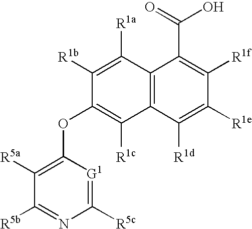
with a chlorinating agent; and
(b) reacting the corresponding product with H₂N—R⁴.

53. The method of claim 52, wherein the chlorinating agent is selected from the group consisting of thionyl chloride, oxalyl chloride, and chlorine.

54. The method of claim 52, wherein the carboxylic acid having the structure: 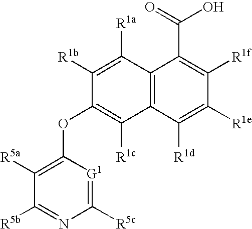
is produced by a method comprising reacting a compound having the formula 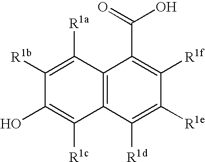
with a compound having the formula 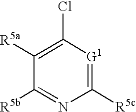
in the presence of a base.

55. A method of producing a compound having the formula of claim 14, comprising reacting an amide having the structure 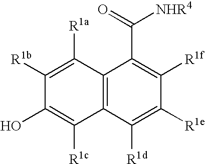
with a compound having the formula 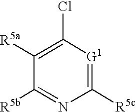
in the presence of a base.
